SEQUENCE LISTING (7 sequences)

SEQ ID NO: 1 - protein, 519 aa, Artificial Sequence
  Vect_G4S_Peg15-2 (anti-EGFR)
MGWSCIILFLVATATGVHSHHHHHHQLQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKSGGGGSEVKLEESGGGLVQPGGSMKLSCVASGFTFSNYWMNWVRQSPEKGLEWVTEIRSKSNNYATHYAESVKGRFTISRDDSKGSVYLQMNNLRAEDTGIYYCSNRYYWGQGTLVTVSAGGGGSGGGGSGGGGSDIVMTQSHKFMSTSVRDRVTITCKASQDVNTSVAWYQQKPGQSPKLVIYWASTRHTGVPDRFTGSGSGTDFTLTISNVQSEDLADYFCLQYINYPYTFGGGTKLEIKEQKLISEEDLN

SEQ ID NO: 2 - protein, 517 aa, Artificial Sequence
  ATX_G4S_PEG15-2 (anti-mesothelin)
MGWSCIILFLVATATGVHSHHHHHHQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGSGTPVTVSSGGGGSGGGGSGGGGSIELTQSPAIMSASPGEKVTMTCSASSSVSYMHWYQQKSGTSPKRWIYDTSKLASGVPGRFSGSGSGNSYSLTISSVEAEDDATYYCQQWSKHPLTFGSGTKVEIKSGGGGSEVKLEESGGGLVQPGGSMKLSCVASGFTFSNYWMNWVRQSPEKGLEWVTEIRSKSNNYATHYAESVKGRFTISRDDSKGSVYLQMNNLRAEDTGIYYCSNRYYWGQGTLVTVSAGGGGSGGGGSGGGGSDIVMTQSHKFMSTSVRDRVTITCKASQDVNTSVAWYQQKPGQSPKLVIYWASTRHTGVPDRFTGSGSGTDFTLTISNVQSEDLADYFCLQYINYPYTFGGGTKLEIKEQKLISEEDLN

SEQ ID NO: 3 - protein, 517 aa, Artificial Sequence
  4B3_G4S_PEG15-2 (anti-Eph2a)
MGWSCIILFLVATATGVHSHHHHHHEVQVQQSGPELVKPGASVKISCKASGYTFTDYDMNWMKQSHGKSLEWIGDINPNNGGASYNQKFRGKATLTVDKSSSTAYMELRSLTSEDSAVYYCARRSTMTYFDYWGQGTTLTVSSGGGGSGGGGSGGGGSQIVLTQSPAIMSASPGEKVTISCSASSSVSYMYWYQQKPGSSPKPWIYRTSNLASGVPARFSGSGSGTSYSLTISTMEAEDAATYYCQQYHSYPLTFGAGTKLELKSGGGGSEVKLEESGGGLVQPGGSMKLSCVASGFTFSNYWMNWVRQSPEKGLEWVTEIRSKSNNYATHYAESVKGRFTISRDDSKGSVYLQMNNLRAEDTGIYYCSNRYYWGQGTLVTVSAGGGGSGGGGSGGGGSDIVMTQSHKFMSTSVRDRVTITCKASQDVNTSVAWYQQKPGQSPKLVIYWASTRHTGVPDRFTGSGSGTDFTLTISNVQSEDLADYFCLQYINYPYTFGGGTKLEIKEQKLISEEDLN

SEQ ID NO: 4 - protein, 486 aa, Artificial Sequence
  scVEGF_G4S_PEG15-2
MGWSCIILFLVATATGVHSHHHHHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDGSTSGSGKSSEGKGEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDSGGGGSEVKLEESGGGLVQPGGSMKLSCVASGFTFSNYWMNWVRQSPEKGLEWVTEIRSKSNNYATHYAESVKGRFTISRDDSKGSVYLQMNNLRAEDTGIYYCSNRYYWGQGTLVTVSAGGGGSGGGGSGGGGSDIVMTQSHKFMSTSVRDRVTITCKASQDVNTSVAWYQQKPGQSPKLVIYWASTRHTGVPDRFTGSGSGTDFTLTISNVQSEDLADYFCLQYINYPYTFGGGTKLEIKEQKLISEEDLN

SEQ ID NO: 5 - protein, 518 aa, Artificial Sequence
  L19.3Hu_G4S_Peg15-2 (anti-CD171-PEG(15-2) BsAb)
MGWSCIILFLVATATGVHSHHHHHHEVQLVQSGGGLVQSGGSLRLSCEQSGEEFERYWMLWVRQRPGHGLEWVGEINPRNDRTNYNEKFKTRFTISVDRSKSTAYLQMDSLRAEDTAVYFCALGGGYAMDYWGQGTLVTVSSGGGGSGGGGSGGGGSDIQMTQSPSSLSASVGDRVTITCRASQDISNYLNWYQQKPGKAPKLLIYYTSRLHSGVPSRFSGSGSGTDYTLTISSLQPEDFATYFCQQGNTLPWTFGGGTKLEIKRSGGGGSEVKLEESGGGLVQPGGSMKLSCVASGFTFSNYWMNWVRQSPEKGLEWVTEIRSKSNNYATHYAESVKGRFTISRDDSKGSVYLQMNNLRAEDTGIYYCSNRYYWGQGTLVTVSAGGGGSGGGGSGGGGSDIVMTQSHKFMSTSVRDRVTITCKASQDVNTSVAWYQQKPGQSPKLVIYWASTRHTGVPDRFTGSGSGTDFTLTISNVQSEDLADYFCLQYINYPYTFGGGTKLEIKEQKLISEEDLN

SEQ ID NO: 6 - protein, 518 aa, Artificial Sequence
  CD200_G4S_Peg15-2 (anti-CD200-PEG(15-2) BsAb)
MGWSCIILFLVATATGVHSHHHHHHQVQLQQSGSELKKPGASVKISCKASGYSFTDYIILWVRQNPGKGLEWIGHIDPYYGSSNYNLKFKGRVTITADQSTTTAYMELSSLRSEDTAVYYCGRSKRDYFDYWGQGTTLTVSSGGGGSGGGGSGGGGSDIQMTQSPSSLSASIGDRVTITCKASQDINSYLSWFQQKPGKAPKLLIYRANRLVDGVPSRFSGSGSGTDYTLTISSLQPEDFAVYYCLQYDEFPYTFGGGTKLEIKRSGGGGSEVKLEESGGGLVQPGGSMKLSCVASGFTFSNYWMNWVRQSPEKGLEWVTEIRSKSNNYATHYAESVKGRFTISRDDSKGSVYLQMNNLRAEDTGIYYCSNRYYWGQGTLVTVSAGGGGSGGGGSGGGGSDIVMTQSHKFMSTSVRDRVTITCKASQDVNTSVAWYQQKPGQSPKLVIYWASTRHTGVPDRFTGSGSGTDFTLTISNVQSEDLADYFCLQYINYPYTFGGGTKLEIKEQKLISEEDLN

SEQ ID NO: 7 - protein, 520 aa, Artificial Sequence
  MUC1_G4S_Peg15-2 (anti-CD227-PEG(15-2) BsAb)
MGWSCIILFLVATATGVHSHHHHHHQVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSSGGGGSGGGGSGGGGSDIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIKSGGGGSEVKLEESGGGLVQPGGSMKLSCVASGFTFSNYWMNWVRQSPEKGLEWVTEIRSKSNNYATHYAESVKGRFTISRDDSKGSVYLQMNNLRAEDTGIYYCSNRYYWGQGTLVTVSAGGGGSGGGGSGGGGSDIVMTQSHKFMSTSVRDRVTITCKASQDVNTSVAWYQQKPGQSPKLVIYWASTRHTGVPDRFTGSGSGTDFTLTISNVQSEDLADYFCLQYINYPYTFGGGTKLEIKEQKLISEEDLN

(19) United States
(12) Patent Application Publication
(10) Pub. No.: US 2018/0016352 A1
(43) Pub. Date: Jan. 18, 2018

(54) TARGETING CONSTRUCTS FOR DELIVERY OF PAYLOADS

(71) Applicant: THE UNIVERSITY OF QUEENSLAND, St. Lucia (AU)

(72) Inventors: Kristofer THURECHT, Loganholme (AU); Christopher HOWARD, Rochedale South (AU); Stephen MAHLER, Wynnum (AU)

(21) Appl. No.: 15/548,942

(22) PCT Filed: Feb. 5, 2016
(86) PCT No.: PCT/AU2016/050069

§ 371 Date: Aug. 4, 2017

(30) Foreign Application Priority Data
Feb. 5, 2015 (AU) 2015900351

(51) Int. Cl. C07K 16/44; C07K 16/28; A61K 49/00; C07K 16/40; G01N 33/545; C07K 16/30

(52) U.S. Cl. CPC C07K 16/44; G01N 33/545; C07K 16/3092; A61K 47/60; A61K 49/0054; A61K 49/0058; C07K 16/40; A61K 47/6851; C07K 16/2863; C07K 16/2803; C07K 16/28; C07K 2317/31; C07K 2317/33; C07K 2317/622; C07K 2317/92

(57) ABSTRACT

Disclosed are targeting constructs for targeting a target site, including a molecule or complex of interest. The targeting constructs comprise a polymer chain and a plurality of multi-specific molecules each of which includes an affinity moiety for binding with the polymer chain and a targeting ligand for binding with the target site. The polymer chain may be associated with a therapeutic or diagnostic agent and generally comprises a plurality of sites, wherein individual sites bind with a multi-specific molecule of the target construct. The present disclosure also relates to methods of preparing the targeting construct and to its use in therapeutic and diagnostic applications.

### FIELD OF THE INVENTION

This application claims priority to Australian Provisional Application No. 2015900351 entitled “Targeting constructs for delivery of payloads”, filed on 5 Feb. 2015, the entire content of which is hereby incorporated by reference herein.

This invention relates generally to a targeting construct for targeting a target site. The targeting construct comprises a polymer chain and a plurality of multi-specific molecules each of which includes an affinity moiety for binding with the polymer chain and a targeting ligand for binding with the target site. The polymer chain may be associated with a therapeutic or diagnostic agent and generally comprises a plurality of sites, wherein individual sites bind with a multi-specific molecule of the target construct. The present invention also relates to methods of preparing the targeting construct and to its use in therapeutic and diagnostic applications.

### BACKGROUND OF THE INVENTION

Polymers and polymeric nanomaterials have significantly altered our perception of what constitutes a therapeutic entity. This is because polymers can change not only the physicochemical properties of a therapeutic substance, but also the physiological interactions with the target cells. The emergence of polymeric materials in nanomedicine is exemplified by the fact that 2 of the top 10 best-selling drugs in the United States in 2013 were polymeric drugs. While the role that polymers play in modulating the therapeutic window of a particular substance is well understood, significant hurdles remain for translating polymeric materials into mainstream pharmaceutics. Of particular note is the often complicated and/or instability of ligation chemistries that are adopted in polymer conjugates.

Several nanomedicine-based formulations are utilized in clinical treatments, with the standout therapeutic formulation that utilizes polymers in cancer therapy being Doxil (Barenholz, Y., Journal of Controlled Release, 2012, 160, 117-134). Doxil improves therapeutic efficacy of the commonly utilized Doxorubicin by increasing its physiological half-life and improving accumulation in tumor tissue via the enhanced permeation and retention (EPR) effect. The increased permeability of tumor vasculature and poor lymphatic drainage leads to enhanced delivery of therapeutic to the cancer. Most nanomedicines in clinical use utilize the EPR effect to passively target tumors, but whilst the EPR effect improves nanoparticle accumulation in tumor tissue, their uptake of the nanoparticles into tumor cells is often poor. This can be overcome by attaching to the exterior of the nanoparticle targeting ligands that specifically bind to cell surface receptors. Depending on the receptor, internalization can occur via receptor mediated endocytosis and this can enhance the therapeutic effect.

Despite the observation that active targeting of nanoparticles with ligands such as antibodies enhances uptake and efficacy in pre-clinical models, polymeric formulations that have progressed to clinical use typically do not employ an active targeting approach. This is because in many cases the complexities associated with ligand conjugation, formulation, up-scaling and industrial exploitation are prohibitive (Lammers, T., et al., Journal of Controlled Release, 2012, 161, 175-187). From a chemistry and nanomaterials viewpoint, strategies to ligate antibodies to polymeric carriers would typically make use of amide formation (via NHS activated acid groups on the nanomaterial with amine groups on the antibody/peptide) but this reaction leads to binding of the nanomaterial to random amine functionality on the antibody, with minimal control over site specificity. An alternative strategy utilizes maleimide coupling to thiols on the antibody, but this often requires mild reduction of disulphide bridges on the antibody that could affect binding potential. Recent use of enzymatic approaches to ligation has allowed site-specific, efficacious conjugation between large molecules (Leung, K. M., et al., Angewandte Chemie (International ed. in English), 2012, 51, 7132-7136), but still requires synthesis of enzyme recognition sites on both moieties adding significant complexity to the final nanomedicine formulation. In all cases, the coupling of large nanomaterials to antibodies in a site-selective manner has proven somewhat challenging (Bouchard, H., et al., Bioorganic & Medicinal Chemistry Letters, 2014, 24(4), 1071-1074).

The challenges associated with current ligation strategies have been recently exemplified in a number of clinical trials. MLN591 is a prostate-specific membrane antigen-directed immunoconjugate for delivering chemotherapeutics to prostate cancer and was trialed in a number of patients in the United States by Millennium Pharmaceuticals. While the drug showed efficacy against the tumor, the authors report “Deconjugation of MLN2704 was evident . . . ” and ultimately progression past the phase 1 trials did not proceed owing to lack of stable linker chemistry (Galsky, M. D., et al., Journal of Clinical Oncology, 2008, 26, 2147-2154). This clearly highlights the requirement for not only improved linker chemistries between targeting moieties and drugs/imaging modalities, but also greater fundamental insight into the effects of the physiological environment on antibody conjugates.

Accordingly, the conjugation steps currently available for attaching targeting molecules such as antibodies or peptides to the surface of nanoparticles have several drawbacks, including variable efficiency, potential to alter the structure and functionality of the targeting molecules, and poor control and quantification over the localization of the targeting molecules on the nanoparticle.

From the foregoing, a need exists for alternative strategies with improved efficiencies for conjugating targeting molecules to polymeric nanomaterials.

### SUMMARY OF THE INVENTION

The present invention is based on the development of novel targeting constructs that comprise a polymer chain and a plurality of multi-specific molecules that are capable of binding to the polymer chain and to a target site. The polymer chain may be associated with a therapeutic or diagnostic agent and generally comprises a plurality of sites, individual ones of which bind with a multi-specific molecule of the targeting construct. In advantageous embodiments, the targeting constructs are prepared in a single step simply by contacting the polymer chain with the plurality of multi-specific molecules. This enables facile binding of the polymer chain, which is suitably conjugated with a therapeutic or imaging agent, to multi-specific molecules that have specificity for a target site of interest. The targeting constructs of the invention have a range of therapeutic and diagnostic applications, as described hereafter.

Accordingly, in one aspect, the present invention provides a targeting construct represented by formula (I):

p-[α-L-λ]ₙ (I)

wherein:

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group; and

λ, independently for each occurrence, represents a targeting ligand; and

n represents an integer of at least 2,

wherein the polymer chain comprises a plurality of affinity moiety-binding partners, wherein individual affinity moiety-binding partners bind with the affinity moiety of a respective multi-specific molecule.

Another aspect of the present invention provides a targeting construct for targeting a payload to a target site, which is represented by formula (II):

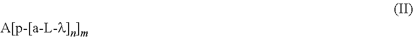

wherein:

A represents the payload;

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group; and

λ, independently for each occurrence, represents a targeting ligand that targets the construct to the target site; and

n represents an integer of at least 2; and

m represents an integer of at least 1,

wherein the polymer chain comprises a plurality of affinity moiety-binding partners, wherein individual affinity moiety-binding partners bind with the affinity moiety of a respective multi-specific molecule.

Suitably, m represents an integer in the range of 1 to 30000 suitably, ≦25000, ≦20000, ≦15000, ≦10000, ≦5000, ≦1000, ≦500, ≦100, ≦50, ≦10, or even ≦5.

In yet another aspect, the present invention provides a method of constructing a targeting construct, the method comprising contacting a polymer chain (p) with a plurality of multi-specific constructs represented by formula (III):

α-L-λ (III)

wherein:

α, independently for each occurrence, represents an affinity moiety that binds with the polymer chain;

L, independently for each occurrence, is absent or represents a linker group; and

λ, independently for each occurrence, represents a targeting ligand;

to thereby form a targeting construct represented by formula (I):

p-[α-L-λ]ₙ (I)

wherein

p, α-L-λ, α, L and λ are as defined above; and

n represents an integer of at least 2,

wherein the polymer chain comprises a plurality of affinity moiety-binding partners, wherein individual affinity moiety-binding partners bind with the affinity moiety of a respective multi-specific molecule.

Still another aspect of the present invention provides a method of constructing a targeting construct for targeting a payload to a target site, the method comprising contacting a conjugate represented by formula (IV):

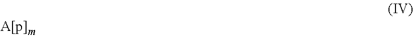

wherein:

A represents the payload;

p represents a polymer chain; and

m represents an integer of at least 1,

with a plurality of multi-specific molecules represented by formula (III):

α-L-λ (III)

wherein:

α, independently for each occurrence, represents an affinity moiety that binds with the polymer chain;

L, independently for each occurrence, is absent or represents a linker group; and

λ, independently for each occurrence, represents a targeting ligand that targets the construct to the target site,

to thereby form a targeting construct represented by formula (II):

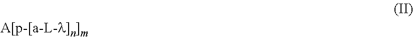

wherein:

A, p, α-L-λ, α, L, λ, n and m are as defined above,

wherein the polymer chain comprises a plurality of affinity moiety-binding partners, wherein individual affinity moiety-binding partners bind with the affinity moiety of a respective multi-specific molecule.

In a further aspect, the present invention provides a method of delivering a payload to a target site in a subject, the method comprising administering to the subject a targeting construct represented by formula (II):

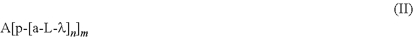

wherein:

A represents the agent;

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group; and

λ, independently for each occurrence, represents a targeting ligand that targets the construct to the target site; and

n represents an integer of at least 2; and

m represents an integer of at least 1,

wherein the polymer chain comprises a plurality of affinity moiety-binding partners, wherein individual affinity moiety-binding partners bind with the affinity moiety of a respective multi-specific molecule.

Yet another aspect of the present invention provides a method for treatment of a subject with a therapeutic agent, wherein the therapeutic agent requires delivery to a target site in the subject, which target site is suitably associated with a condition to be treated, the method comprising administering to the subject an effective amount of a targeting construct represented by formula (II):

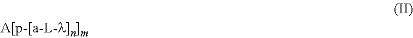

wherein:

A represents the therapeutic agent;

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group; and

λ, independently for each occurrence, represents a targeting ligand that targets the construct to the target site; and

n represents an integer of at least 2; and

m represents an integer of at least 1,

wherein the polymer chain comprises a plurality of affinity moiety-binding partners, wherein individual affinity moiety-binding partners bind with the affinity moiety of a respective multi-specific molecule.

Another aspect of the present invention provides a method for imaging a target site in a subject, the method comprising administering to the subject a targeting construct represented by formula (II):

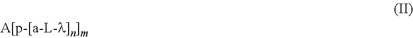

wherein:

A represents an imaging agent;

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group; and

λ, independently for each occurrence, represents a targeting ligand that targets the construct to the target site; and

n represents an integer of at least 2; and

m represents an integer of at least 1,

wherein the polymer chain comprises a plurality of affinity moiety-binding partners, wherein individual affinity moiety-binding partners bind with the affinity moiety of a respective multi-specific molecule.

In still another aspect of the present invention, a method is provided for modulating the activity of a target molecule or complex, the method comprising contacting the target molecule or complex with a targeting construct represented by formula (Ia) or formula (IIa):

p-[α-L-λ]ₙ (Ia)

wherein:

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group; and

λ, independently for each occurrence, represents a targeting ligand that targets the construct to the target molecule or complex; and

n represents an integer of at least 2,

wherein the polymer chain comprises a plurality of affinity moiety-binding partners, wherein individual affinity moiety-binding partners bind with the affinity moiety of a respective multi-specific molecule,

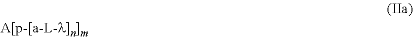

wherein:

A represents the payload;

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group;

λ, independently for each occurrence, represents a targeting ligand that targets the construct to the target molecule or complex;

n represents an integer of at least 2; and

m represents an integer of at least 1,

wherein the polymer chain comprises a plurality of affinity moiety-binding partners, wherein individual affinity moiety-binding partners bind with the affinity moiety of a respective multi-specific molecule, and

wherein the targeting ligands of individual multi-specific molecules of the construct bind with, and thereby modulate the activity of, the target molecule or complex.

Yet another aspect of the present invention provides a method for detecting a target analyte, the method comprising contacting the target analyte with a targeting construct represented by formula (Ib):

p-[α-L-λ]ₙ (Ib)

wherein:

p represents a polymer chain;

α-L-λ, independently for each occurrence, represents a multi-specific molecule,

wherein:

α, independently for each occurrence, represents an affinity moiety that binds with the polymer chain;

L, independently for each occurrence, is absent or represents a linker group; and

λ, independently for each occurrence, represents a targeting ligand that binds with the target analyte; and

n represents an integer of at least 2,

wherein the polymer chain comprises a plurality of affinity moiety-binding partners, wherein individual affinity moiety-binding partners bind with the affinity moiety of a respective multi-specific molecule,

to thereby form a complex comprising the targeting construct and the target analyte.

### BRIEF DESCRIPTION OF THE DRAWINGS

FIG. 1 shows sequence information for certain embodiments of the multi-specific molecules of the invention, representing pi-specific antibodies (BsAbs). Text in italics indicates a secretion signal. Underlined text on a white background represents 6xHis and underlined text on a gray background represents a c-Myc affinity tag. Regular text on a white background indicates cancer-targeting scFv and regular text on a gray background represents methoxy polyethylene glycol (mPEG) targeting scFv.

FIG. 2 is a graphical representation showing an ELISA based determination of binding specificity of PEG BsAbs to recombinant target receptors EphA2, VEGFR2, Mesothelin and EGFR. BsAbs were screened from culture supernatant, so different responses are relative to levels of BsAb expressed. A: 4B3-15.2 is a PEG BsAb targeting EphA2; B: VEGF-15.2 is a BsAb targeting VEGFR2; C: ATX-15.2 is a BsAb targeting mesothelin, and ID: Vect-15.2 is a BsAb targeting EGFR. All BsAbs have an anti-PEG antibody fragment (15.2) fused via a G4S (Gly-Gly-Gly-Ser) linker to anti-receptor antibody fragments (4B3, ATX, Vect) or ligand (VEGF),

FIG. 3 is a graphical representation showing an ELISA based determination of binding specificity of PEG BsAbs to polyethylene glycol (PEG) monomethyl ether methacrylate based polymer. Binding is based on absorbance values at 450 nm. BsAbs were screened from culture supernatant, so different responses are relative to levels of BsAb expressed, A: 4B3-15.2 is a PEG BsAb targeting EphA2: B: VEGF-15.2 is a BsAb targeting VEGFR2; C: ATX-15.2 is a BsAb targeting mesothelin; and D: Vect-15.2 is a BsAb targeting EGFR. All BsAbs have an anti-PEG antibody fragment (15.2) fused via a G4S (Gly-Gly-Gly-Ser) linker to anti-receptor antibody fragments (4B3, ATX, Vect) or ligand (VEGF). Vect-1H10 represents a BsAb that binds to EGFR and a non-pegylated nanoparticle. Histogram demonstrates binding by all anti-PEG BsAbs to PEG polymer, but no binding by a BsAb not targeting PEG.

FIG. 4 is a graphical representation showing a competitive binding ELISA. 10 μg/mL of BsAbs were pre-incubated with various concentrations (48, 4.8, 0.48 μg/mL) of PEG polymer and the binding of the BsAb-PEG complexes to solid phase immobilized PEG polymer was evaluated. Results indicate that 48 μg/mL of polymer completely saturates 10 μg/mL BsAbs with the exception of the anti mesothelin PEG BsAb (ATX-15.2) which still retains the ability to bind immobilized polymer,

FIG. 5 is a graphical representation showing flow cytometry data demonstrating binding of Cy5 labeled PEG polymer (blue; left curve), Cy5 labeled PEG-non specific BsAb conjugate (green; left curve), and Cy5 labeled PEG-anti-EGFR BsAb conjugate (red; right curve) to MDA-MB-468 cells. There is no binding of Cy5 PEG alone (blue; left curve) or Cy5 PEG-non specific BsAb conjugate (Green) to the cells.

FIG. 6 is a photographic representation showing fluorescence imaging of hyperbranched Pegylated polymer labeled with Cy-5 fluorophore into a xenograft glioma mouse model. The mouse on the left was injected with the polymer following 30 min incubation with anti-PEG-anti-EphA2 bispecific, while the mouse on the right was injected with polymer only. Images are 24 hr post-injection. Clearly the polymer targeted to the EphA2 receptor (highly overexpressed in the U87 tumor cells) shows higher uptake than the untargeted control mouse where only polymer was injected.

FIG. 7 is a schematic representation illustrating: a) the binding of a plurality of BsAbs to pendant PEG groups of a PEG-methacrylate co-polymer chain; and b) that an increase in the number of affinity moiety-binding partners on a polymer chain that can bind to an anti-PEG BsAb increases the avidity for the anti-PEG BsAb for the polymer chain as well increasing the density of targeting ligand.

FIG. 8 is a schematic representation showing biolayer interferometry (BLI) analysis of EGFR-mPEG BsAb binding affinities for recombinant EGFR and mPEG. (A) Schematic of BLI method for measuring EGFR-mPEG BsAb binding affinity (kDa) for hyperbranched PEG. HBP is coated on aminopropylsilane (APS) biosensors (green bar), then free sites are blocked with Bovine serum albumin (BSA; orange bar). EGFR-mPEG at a range of concentrations (500, 250, 125, 62.5 nM) are added to HBP coated sensors (red bar) and association rate measured. Sensors are added to PBS (blue bar) to measure dissociation rates. EGFR-LPS at same concentrations is used as a negative. (B) Binding of EGFR-mPEG to HBP and linear mPEG. There is a 6-fold increase in the BsAb binding response to HBP (blue line) compared to linear mPEG (yellow line). There is no binding of an alternative EGFR-PEG BsAb to HBP or linear mPEG. (C) BLI kinetics curves for EGFR-mPEG binding to recombinant EGFR. (D) BLI kinetics curves for EGFR-mPEG binding to HBP.

FIG. 9 shows sequence information for certain embodiments of the multi-specific molecules of the invention, representing bi-specific antibodies (BsAbs) with specificity to mPEG and to three novel cancer cell surface antigens, CD171 (L1 cell adhesion molecule; L1CAM), CD200 (OX-2 membrane glycoprotein; OX-2), and CD227 (mucin 1; MUC1). Text in italics indicates a secretion signal. Underlined text on a white background represents 6xHis and underlined text on a gray background represents a c-Myc affinity tag. Regular text on a white background indicates cancer cell surface-targeting scFv and regular text on a gray background represents mPEG targeting scFv.

FIG. 10 is a graphical representation showing ELISA analysis or anti-CD171-PEG BsAb binding to immobilized nanoparticle and recombinant receptors. The reference 96-Well plates were coated with 10 μg/mL receptor/nanoparticle and exposed to a concentration of 100 μg/mL of BsAb. Binding was assayed using anti-c-myc HRP and TMB. Absorbance readings (450 nm) were plotted using Graph Pad Prism, Anti-CD171-PEG BsAb shows specific binding to both CD171 and the PEG-nanoparticle in the absence (A) and presence (B) of Tween 20.

FIG. 11 is a graphical representation showing a dose response curve of anti-CD171-PEG BsAb binding to immobilized nanoparticle, 96-Well plates were coated with 10 μg/mL recombinant CD171 and exposed to a concentration gradient of BsAb. Binding was assayed using anti-c-rnyc HRP and TMB. Absorbance readings (450 nm) were plotted using Graph Pad Prism. A line of best fit was calculated using nonlinear regression analysis and was then plotted (R2=0.9831). Error shown as SD, from this line of best fit the EC50 was determined to be 14.97±4.16 pM.

FIG. 12 is a graphical representation showing analysis of anti-CD171-PEG BsAb PEG-nanoparticle interaction by Dynamic Light Scatter analysis. Backscatter angle 173 degrees; Dispersant: 0.5M Ned 0.1M arginine, dispersarit viscosity 0.9286 mPa/s, dispersant refractive index 1.349; Sample: Protein, refractive index 1.450, absorbance 0.001. Addition of PEG-Nanoparticle to anti-CD171-PEG BsAb correlates in a dose dependent manner with an increase in average particle size (d.nm); A: anti-CD171-PEG BsAb only (10.1 nm); B: anti-CD171-PEG BsAb+1 μg Nanoparticle (11.7 nm); C: anti-CD171-PEG BsAb+10 μg nanoparticle (15.7 nm); D: anti-CD171-PEG BsAb+100 μg nanoparticle (11.3 nm and 43.8 nm); and E: 100 μg nanoparticle only (5.9 nm).

FIG. 13 is a graphical representation showing flow cytometry analysis of anti-myc FITC labeled BsAb binding to SKOV-3 cells. SKOV-3 cells were grown to confluency in Advanced RPMI 1640 medium supplemented with 1× Glutamax and 10% FCS and then scraped, Cells were then incubated for 1 hr on ice with either anti-c-myc FITC, anti-c-myc FITC and anti-CD171-PEG BsAb, anti-c-myc FITC and anti-CD200-PEG BsAb or anti-c-myc FITC and anti-CD227-PEG BsAb in PBSFCS (PBS+10% FCS). Following incubation, cells were washed with PBSFCS to remove unbound antibody and nanoparticle. Fluorescence was then assayed using a BD LSR II Analyzer and data were analyzed using Flowing 2.1. Panel A (Count/Absorbance 530 nm) shows no shift in FITC fluorescence, in the presence of anti-myc FITC antibody alone. Panel B (Count/Absorbance 530 nm) shows a shift in FITC fluorescence, in the presence of anti-myc FITC antibody and anti-CD171-PEG BsAb. This is indicative of BsAb binding to the cell. Panel C (Count/Absorbance 530 nm) shows no shift in FITC fluorescence, in the presence of anti-myc FITC antibody and anti-CD200-PEG BsAb. Panel D (Count/Absorbance 530 nm) shows no shift in FITC fluorescence, in the presence of anti-myc FITC antibody and anti-CD227-PEG BsAb

FIG. 14 is a graphical representation showing flow cytometry analysis of anti-myc FITC labeled BsAb binding to MDA-MB-468 cells. MDA-MB-468 cells were grown to confluency in Advanced RPMI 1640 medium supplemented with lx Glutamax and 10% FCS and then scraped. Cells were then incubated for 1 hr on ice with either anti-c-myc FITC, anti-c-myc FITC and anti-EGFR-PEG BsAb, anti-c-myc FITC and anti-CD171-PEG BsAb, anti-c-myc FITC and anti-CD200-PEG BsAb or anti-c-myc FITC and anti-CD227-PEG BsAb in PBSFCS (PBS+10% FCS). Following incubation cells were washed with PBSFCS to remove unbound antibody and nanoparticle. Fluorescence was then assayed using a BD LSR II Analyser and data was analysed using Flowing 2.1. Panel A (Count/Absorbance 530 nm) shows no shift in FITC fluorescence, in the presence of anti-myc FITC antibody alone. Panel B (Count/Absorbance 530 nm) shows a shift in FITC fluorescence, in the presence of anti-myc FITC antibody and anti-EGFR-PEG BsAb. This is indicative of binding of BsAb to cells. Panel C (Count/Absorbance 530 nm) shows no shift in FITC fluorescence, in the presence of anti-myc FITC antibody and anti-CD171-PEG BsAb. Panel D (Count/Absorbance 530 nm) shows no shift in FITC fluorescence, in the presence of anti-myc FITC antibody and anti-CD200-PEG BsAb. Panel E (Count/Absorbance 530nm) shows no shift in FITC fluorescence, in the presence of anti-myc FITC antibody and anti-CD227-PEG BsAb.

FIG. 15 is a graphical representation showing ELISA analysis of anti-CD200-PEG BsAb binding to immobilized nanoparticle and recombinant receptors. 96-Well plates were coated with 10 μg/mL receptor/nanoparticle and exposed to a concentration of 100 μg/mL of BsAb. Binding was assayed using anti-c-myc HRP and TMB. Absorbance readings (450 nm) were plotted using Graph Pad Prism. Anti-CD200-PEG BsAb shows specific binding to both CD200 and the PEG-nanoparticle in the absence of Tween 20 (A). (B) Binding to PEG-nanoparticle is compromised in the presence of Tween 20.

FIG. 16 is a graphical representation showing a dose response curve of anti-CD200-PEG BsAb binding to immobilized nanoparticle. 96-Well plates were coated with 10 μg/mL recombinant CD200 and exposed to a concentration gradient of BsAb. Binding was assayed using anti-c-myc HRP and TMB. Absorbance readings (450 nm) were plotted using Graph Pad Prism. A line of best fit was calculated using nonlinear regression analysis and was then plotted (R2=0.9866). Error shown as SD, from this line of best fit the EC50 was determined to be 37.66±8.25 pM.

FIG. 17 is a graphical representation showing ELISA analysis of anti-CD227-PEG BsAb binding to immobilized nanoparticle and recombinant receptors. 96-Well plates were coated with 10 μg/mL receptor/nanoparticle and exposed to a concentration of 100 μg/mL of BsAb. Binding was assayed using anti-c-myc HRP and TMB. Absorbance readings (450 nm) were plotted using Graph Pad Prism. Anti-CD227-PEG BsAb shows specific binding to the PEG-nanoparticle, but not CD227 in the absence of Tween 20 (A). (B) Binding to PEG-nanoparticle is compromised in the presence of Tween 20.

FIG. 18 a graphical representation showing a dose response curve of anti-CD227-PEG BsAb binding to immobilized nanoparticle. 96-Well plates were coated with 10 μg/mL recombinant CD227 and exposed to a concentration gradient of BsAb. Binding was assayed using anti-c-myc HRP and TMB. Absorbance readings (450 nm) were plotted using Graph Pad Prism. A line of best fit was calculated using nonlinear regression analysis and was then plotted (R2=0.8789). Error shown as SD, from this line of best fit the EC50 was determined to be approximately 59.66 nM.

FIG. 19 is a graphical representation showing characterization of EGFR-mPEG BsAb-HBP bionanomaterial. (A) Dynamic light scattering (DLS) measuring number mean particle size of BsAb (red; 8 nm), HBP (blue; 10 nm) and BsAb-HBP mix (green; 23 nm). (B) DLS measuring the change in number mean particle size following mixing of 2000 nM BsAb (black; 10 nm) with 10 nM HBP (red; 12 nm), 100 nM HBP (green; 23 nm) and 1000 nM HBP (blue; 30 nm). (C) Biolayer interferometry (BLI) demonstrating specific binding of recombinant EGFR to immobilized HBP-BsAb complex. EGFR-mPEG BsAb is bound to immobilized HBP (green bar) for 600 s and then a dissociation (600 s) step is performed to enable binding affinity to be determined for BsAb binding to HBP. Following these steps a new baseline (300 s) step is performed and then rEGFR and EphA2 receptors are added to immobilized BsAb-HBP complexes (red bar) for 600 s. Binding of EGFR is detected but no binding to EphA2. There is no binding of EGFR-LPS BsAb to HBP or receptors.

FIG. 20 is a graphical representation showing bispecific antibody targeting of Cy5-HBP to native EGFR on MDA-MB-468 cells. (A) MDA-MB-468 cells (Red) treated with non-targeted and BsAb targeted Cy5 labelled hyperbranched PEG (Cy5-HBP, Cy5-PEG+EphA2-mPEG/EGFR-mPEG BsAb) were analyzed by flow cytometry for binding of FITC-BsAb at 530 nm (FITC) and binding Cy5-HBP at 660 nm (Red-A/Cy5). (B&C) Histograms representative of FITC BsAb fluorescence on cells at absorbance 530 nm (FITC) and Cy5-HBP fluorescence on cells at absorbance 660nm (Red-A/Cy5) (C) for MDA-MB-468 cells+FITC anti myc with PBS (Gray), Cy5-HBP (Green), Cy5-HBP+EphA2-mPEG BsAb (Yellow) or Cy5-HBP+EGFR-mPEG BsAb (Red). (C) Confocal imaging of MDA-MB-468 cells pre-stained with Pyronin-Y (green) and incubated with Cy5-HBP and EGFR-mPEG targeted Cy5 HBP (both red). (D) Images through the z-volume of cells treated with EGFR-mPEG targeted Cy5-HBP. Co-localization of Cy5 and Pyronin Y can be observed (yellow), white arrows indicate examples of internalized Cy5-HBP.

FIG. 21 is a graphical representation showing a Nyquist diagram displaying layer-by-layer functionalization of SPGE: (i) 1 mM Linear mPEG/MCH monolayer, (ii) 1 mM HBP/MCH monolayer, (iii) EGFR-BsAb (Linear mPEG) and (iv) EGFR-BsAb (HBP). All measurements in 10 mM phosphate buffer containing 2.5 mM K₃[Fe(CN)₆], 2.5 mM K₂[Fe(CN)₆] and 0.1 M KCl.

Some figures and text contain color representations or entities. Color illustrations are available from the Applicant upon request or from an appropriate Patent Office. A fee may be imposed if obtained from a Patent Office.

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

Further, the term “about,” as used herein when referring to a measurable value such as an amount of a compound or agent, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified value.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

As used herein, the term “affinity” refers to the equilibrium constant for the reversible binding of two agents and is expressed as K_{D}. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM).

As used herein the term “affinity moiety”, as used herein, refers to a molecule that binds with an affinity moiety-binding partner (e.g., an epitope, a receptor, a ligand etc.) to form an affinity binding pair. Likewise, the term “affinity moiety-binding partner” refers to a moiety or molecule that binds with an affinity moiety. The affinity moiety can be synthetic, semi-synthetic, or naturally occurring. The binding can occur through non-covalent interactions, such as hydrogen bonds, Van der Waals contacts, Van der Waals/London dispersions, [symbol] stacking and ionic bonds (e.g., salt bridges) or through covalent interactions with the exception of covalent bonds that are targeted by reducing agent capable of cleaving the target covalent bond through addition of hydrogen. Illustrative affinity binding pairs include, for example, avidin or streptavidin and biotin; ligands and receptors; protein A or G binding and Fc-region of immunoglobulin; oligonucleotides and complementary sequences, e.g., polydesoxyadenylic acid and polydesoxythimidylic acid, or polydesoxyguanylic acid and polydesoxycytidylic acid; Ni-NTA (nitrilotriacetic acid, nickel salt) and poly histidine-tagged ligand, and the like. In some embodiments, the “affinity moiety” and “affinity moiety-binding partner” are members of a specific binding pair. A specific binding pair comprises two different moieties/molecules that specifically bind to each other through chemical or physical means. Specific binding partners include antigens/epitopes and their antigen-binding molecules (e.g., antibodies), enzymes and their binding partners (including cofactors, inhibitors and chemical moieties whose reaction the enzymes promote), ligands (e.g., hormones, cytokines, growth factors, vitamins etc.) and their receptors, complementary peptides, specific carbohydrate groups and lectins, antibiotics and their antibodies and naturally occuring binding proteins, complementary nucleotide sequences, aptamers and their targets, biotin and avidin (or streptavidin), and the like. Furthermore, specific binding pairs can include members that are analogs of the original specific binding members, for example, an analyte-analog. Immuno-interactive specific binding members include antigens, antigen fragments and their epitopes, and antigen binding molecules such as but not limited to antibodies, antibody fragments, and variants (including fragments of variants) thereof, whether isolated or recombinantly produced.

As used herein the term “alkyl” refers to a straight or branched chain hydrocarbon having one to twelve carbon atoms, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein the term “alkenyl” refers to a straight or branched chain aliphatic hydrocarbon having two to twelve carbon atoms and containing one or more carbon-to-carbon double bonds, which may be optionally substituted, with multiple degrees of substitution being allowed. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl, cyclopentenyl, cyclohexenyl, cyclohexadienyl, vinyl, and allyl.

As used herein, the term “alkylene” refers to a straight or branched chain divalent hydrocarbon radical having from one to ten carbon atoms, which may be optionally substituted, with multiple degrees of substitution being allowed. Examples of “alkylene” as used herein include, but are not limited to, methylene, ethylene, n-propylene, and n-butylene as well as oxyalkylene groups such as but not limited to oxymethylene, oxyethylene and oxypropylene.

As used herein, the term “alkenylene” refers to a straight or branched chain divalent hydrocarbon radical having from two to ten carbon atoms and containing one or more carbon-to-carbon double bonds, which may be optionally substituted as herein further described, with multiple degrees of substitution being allowed. Examples of “alkenylene” as used herein include, but are not limited to, vinylene, allylene, and 2-propenylene.

As used herein, the term “alkynylene” refers to a straight or branched chain divalent hydrocarbon radical having from two to ten carbon atoms and containing one or more carbon-to-carbon triple bonds, which may be optionally substituted as herein further described, with multiple degrees of substitution being allowed. An example of “alkynylene” as used herein includes, but is not limited to, ethynylene.

As used herein the term “alkynyl” refers to a straight or branched chain aliphatic hydrocarbon having two to twelve carbon atoms and containing one or more carbon-to-carbon triple bonds, which may be optionally substituted as herein further described, with multiple degrees of substitution being allowed. An example of “alkynyl” as used herein includes, but is not limited to, ethynyl.

The term “analyte” is used herein in its broadest sense, to refer without limitation, to a detectable component, such as a substance or chemical constituent in a biological fluid or tissue, including target molecules and complexes, as described for example herein. Analytes can include naturally occurring substances, artificial substances, metabolites, and/or reaction products.

As used herein, “and/or” refers to and encompasses any and all possible combinations of one or more of the associated listed items, as well as the lack of combinations when interpreted in the alternative (or).

The term “antigen” refers to a molecule or a portion of a molecule capable of being bound by a selective binding agent, including antigen-binding molecules as defined for example herein. In some embodiments, the antigen is capable of being used in an animal to produce antibodies capable of binding to that antigen. An antigen can possess one or more epitopes that are capable of interacting with different antigen-binding molecules (e.g., antibodies).

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Aralkyl” means alkyl as defined above which is substituted with an aryl group as defined above, e.g., —CH₂phenyl, —(CH₂)₂phenyl, —(CH₂)₃phenyl,—H₂CH(CH₃)CH₂phenyl, and the like and derivatives thereof.

As used herein, the term “aryl” refers to a monocyclic or fused bicyclic, tricyclic or greater, aromatic ring assembly where each ring contains 3 to 7 atoms, which may be optionally substituted. For example, aryl may be phenyl, benzyl, azulenyl or naphthyl.

“Arylene” means a divalent radical derived from an aryl group. Aryl groups can be mono-, di- or tri-substituted by one, two or three radicals selected from alkyl, alkoxy, aryl, hydroxy, halogen, cyano, amino, amino-alkyl, trifluoromethyl, alkylenedioxy and oxy-C₂-C₃-alkylene; all of which are optionally further substituted. Alkylenedioxy is a divalent substitute attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g., oxyethylene or oxypropylene. Arylene groups include, but are not limited to, phenylene.

As used herein, the term “associated with” refers to the state of two or more entities that are linked by a direct or indirect covalent or non-covalent interaction. In some embodiments, an association is covalent. In some embodiments, a covalent association is mediated by a linker moiety. In some embodiments, an association is non-covalent (e.g., charge interactions, affinity interactions, metal coordination, physical adsorption, host-guest interactions, hydrophobic interactions, [symbol] stacking interactions, hydrogen bonding interactions, van der Waals interactions, magnetic interactions, electrostatic interactions, dipole-dipole interactions, etc.). For example, in some embodiments, an entity (e.g., a payload to be delivered) may be covalently associated with a polymer chain or assembly comprising a plurality of polymer chains. In some embodiments, an entity (e.g., a payload to be delivered) may be non-covalently associated with a polymer chain or assembly comprising a plurality of polymer chains, (e.g., the entity may be associated with the surface of, encapsulated within, surrounded by, and/or distributed throughout an assembly comprising a plurality of polymer chains).

As used herein, the term “assembly” and “polymeric vehicle” are used interchangeably herein to refer to a plurality of interconnected molecules, including a plurality of interconnected polymer chains. In some cases, the polymer chains may be interconnected via bonds, including, for example, covalent bonds (e.g., carbon-carbon, carbon-oxygen, oxygen-silicon, sulfur-sulfur, phosphorus-nitrogen, carbon-nitrogen, metal-oxygen or other covalent bonds), ionic bonds, hydrogen bonds (e.g., between hydroxyl, amine, carboxyl, thiol and/or similar functional groups, for example), dative bonds (e.g., complexation or chelation between metal ions and monodentate or multidentate ligands), or the like. The interaction may also comprise, in some instances, Van der Waals interactions or a binding event between pairs of molecules, such as biological molecules, for example. In some embodiments, the assembly includes particles such as nanoparticles and microparticles,

As used herein, the term “avidity” refers to the resistance of a complex of two or more agents to dissociation after dilution and is generally an informative measure of the overall stability or strength of the complex. In specific embodiments, the complex is one between an affinity moiety (e.g., an antibody) and an epitope of an antigen. In these embodiments, avidity is controlled by three major factors: the affinity of the affinity moiety for the epitope; the valence of both the antigen and affinity moiety; and the structural arrangement of the interacting parts. Ultimately these factors define the specificity of the affinity moiety, that is, the likelihood that the particular affinity moiety is binding to a precise antigen epitope. Avidities can be determined by methods such as a Scatchard analysis or any other technique familiar to one of skill in the art.

The terms “binding”, “attaching”, “conjugating”, “ligating”, “linking”, “tethered” and their grammatical equivalents are used interchangeably herein to refer to the act of connecting or uniting together two or more components (e.g., molecules) by a bond, link, force or tie in order to keep those components together, which encompasses either direct or indirect attachment such that for example where a first compound is directly bound to a second compound, and the embodiments wherein one or more intermediate compounds, and in particular molecules, are disposed between the first compound and the second compound. The components may be connected or united together, for example, by covalent interaction or by non-covalent interaction (e.g., charge interactions, affinity interactions, metal coordination, physical adsorption, host-guest interactions, hydrophobic interactions, [symbol] stacking interactions, hydrogen bonding interactions, van der Waals interactions, magnetic interactions, electrostatic interactions, dipole-dipole interactions, etc.). In specific embodiments, these terms refer to affinity interactions.

The term “biocompatible”, as used herein, refers to any material does not illicit a substantial detrimental response in the host. There is always concern, when a foreign object is introduced into a living body, that the object will induce an immune reaction, such as an inflammatory response that will have negative effects on the host. In the context of this invention, biocompatibility is evaluated according to the application for which it was designed: for example; a bandage is regarded a biocompatible with the skin, whereas an implanted medical device is regarded as biocompatible with the internal tissues of the body. Suitably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “bioerodible” as used herein, refers to any material that is mechanically worn away from a surface to which it is attached without generating any long-term inflammatory effects such that the molecular structure has not been altered. In one sense, bioerosion represents the final stages of “biodegradation” wherein stable low molecular weight products undergo a final dissolution.

The term “biological sample” as described herein, includes any biological specimen that may be extracted, untreated, treated, diluted or concentrated from a subject. Biological samples may include, without limitation, biological fluids such as whole blood, serum, plasma, saliva, urine, tears, sweat, sebum, nipple aspirate, ductal lavage, tumor exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Biological samples may include tissue samples and biopsies, tissue homogenates and the like. The term “biological sample” encompasses samples that have been manipulated in any way after their procurement, such as by treatment with reagents, solubilization, sedimentation, or enrichment for certain components. The term encompasses a clinical sample, and also includes cells in cell culture, cell supernatants and cell lysates. A biological sample may include an analyte.

The term “biostable” as used herein, refers to any material that remains within a physiological environment for an intended duration resulting in a medically beneficial effect.

The term “complex” as used herein refers to a coordination or association of two or more components (e.g., molecules) linked by covalent interactions or by non-covalent interaction (e.g., charge interactions, affinity interactions, metal coordination, physical adsorption, host-guest interactions, hydrophobic interactions, [symbol] stacking interactions, hydrogen bonding interactions, van der Waals interactions, magnetic interactions, electrostatic interactions, dipole-dipole interactions, etc.).

The term “derivatize,” “derivatizing” and the like refer to producing or obtaining a compound from another substance by chemical reaction, e.g., by adding one or more reactive groups to the compound by reacting the compound with a functional group-adding reagent, etc.

By “effective amount”, in the context of treating or preventing a condition is meant the administration of an amount of an agent or composition to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “epitope” refers to a specific immuno-interactive site within an antigen and includes any determinant capable being bound by an antigen-binding molecules as defined for example herein. An epitope is a region of an antigen that is bound by an antigen-binding molecule that targets that antigen. In some embodiments, the antigen is a protein, and an epitope includes specific amino acids that directly bind with the antigen-binding molecule. In other embodiments, the antigen is a non-protein polymer chain, and an epitope includes specific determinants formed by the monomer units of the polymer chain that directly bind with the antigen-binding molecule. Epitope determinants can include chemically active surface groupings of molecules such as amino acids, sugar side chains, phosphoryl or sulfonyl groups, and can have specific three dimensional structural characteristics, and/or specific charge characteristics. Generally, antigen-binding molecules specific for a particular target antigen will preferentially recognize an epitope on the target antigen in a complex mixture of proteins and/or macromolecules.

The term “group” as applied to chemical species refers to a set of atoms that forms a portion of a molecule. In some instances, a group can include two or more atoms that are bonded to one another to form a portion of a molecule. A group can be monovalent or polyvalent (e.g., bivalent) to allow bonding to one or more additional groups of a molecule. For example, a monovalent group can be envisioned as a molecule with one of its hydrogen atoms removed to allow bonding to another group of a molecule. A group can be positively or negatively charged. For example, a positively charged group can be envisioned as a neutral group with one or more protons (i.e., H⁺) added, and a negatively charged group can be envisioned as a neutral group with one or more protons removed. Non-limiting examples of groups include, but are not limited to, alkyl groups, alkylene groups, alkenyl groups, alkenylene groups, alkynyl groups, alkynylene groups, aryl groups, arylene groups, iminyl groups, iminylene groups, hydride groups, halo groups, hydroxy groups, alkoxy groups, carboxy groups, thio groups, alkylthio groups, disulfide groups, cyano groups, nitro groups, amino groups, alkylamino groups, dialkylamino groups, silyl groups, and siloxy groups. Groups such as alkyl, alkenyl, alkynyl, aryl, and heterocyclyl, whether used alone or in a compound word or in the definition of a group may be optionally substituted by one or more substituents. “Optionally substituted,” as used herein, refers to a group may or may not be further substituted with one or more groups selected from alkyl, alkenyl, alkynyl, aryl, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, phenylamino, diphenylamino, benzylamino, dibenzylamino, hydrazino, acyl, acylamino, diacylamino, acyloxy, heterocyclyl, heterocycloxy, heterocyclamino, haloheterocyclyl, carboxy ester, carboxy, carboxy amide, mercapto, alkylthio, benzylthio, acylthio and phosphorus-containing groups. As used herein, the term “optionally substituted” may also refer to the replacement of a CH₂ group with a carbonyl (C═O) group. Non-limiting examples of optional substituents include alkyl, suitably C₁₋₈ alkyl (e.g., C₁₋₆ alkyl such as methyl, ethyl, propyl, butyl, cyclopropyl, cyclobutyl, cyclopentyl or cyclohexyl), hydroxy C₁₋₈ alkyl (e.g., hydroxymethyl, hydroxyethyl, hydroxypropyl), alkoxyalkyl (e.g., methoxymethyl, methoxyethyl, methoxypropyl, ethoxymethyl, ethoxyethyl, ethoxypropyl etc.) C₁₋₈ alkoxy, (e.g., C₁₋₆ alkoxy such as methoxy, ethoxy, propoxy, butoxy, cyclopropoxy, cyclobutoxy), halo (fluoro, chloro, bromo, iodo), monofluoromethyl, monochloromethyl, monobromomethyl, difluoromethyl, dichloromethyl, dibromomethyl, trifluoromethyl, trichloromethyl, tribromomethyl, hydroxy, phenyl (which itself may be further substituted, by an optional substituent as described herein, e.g., hydroxy, halo, methyl, ethyl, propyl, butyl, methoxy, ethoxy, acetoxy, amino), benzyl (wherein the CH₂ and/or phenyl group may be further substituted), phenoxy (wherein the CH₂ and/or phenyl group may be further substituted), benzyloxy (wherein the CH₂ and/or phenyl group may be further substituted), amino, C₁₋₈ alkylamino (e.g., C₁₋₆ alkyl, such as methylamino, ethylamino, propylamino), di C₁₋₈ alkylamino (e.g., C₁₋₆ alkyl, such as dimethylamino, diethylamino, dipropylamino), acylamino (e.g., NHC(O)CH₃), phenylamino (wherein phenyl itself may be further substituted), nitro, formyl, —C(O)—C₁₋₈ alkyl (e.g., C₁₋₆ alkyl, such as acetyl), O—C(O)-alkyl (e.g., C₁₋₆ alkyl, such as acetyloxy), benzoyl (wherein the CH₂ and/or phenyl group itself may be further substituted), replacement of CH₂ with C═O, CO₂H, CO₂ C₁₋₈ alkyl (e.g., C₁₋₆ alkyl such as methyl ester, ethyl ester, propyl ester, butyl ester), CO₂phenyl (wherein phenyl itself may be further substituted), CONH₂, CONHphenyl (wherein phenyl itself may be further substituted), CONHbenzyl (wherein the CH₂ and/or phenyl group may be further substituted),CONH C₁₋₈ alkyl (e.g., C₁₋₆ alkyl such as methyl amide, ethyl amide, propyl amide, butyl amide), CONH C₁₋₈ alkylamine (e.g., C₁₋₆ alkyl such as aminomethyl amide, aminoethyl amide, aminopropyl amide, aminobutyl amide), —C(O)heterocyclyl (e.g., —C(O)-1-piperidine, —C(O)-1-piperazine, —C(O)-4-morpholine), —C(O)heteroaryl (e.g., —C(O)-1-pyridine, —C(O)-1-pyridazine, —C(O)-1-pyrimidine, —C(O)-1-pyrazine), CONHdi C₁₋₈ alkyl (e.g., C₁₋₆alkyl).

“Heteroaralkyl” group means alkyl as defined above which is substituted with a heteroaryl group, e.g., —CH₂pyridinyl, —(CH₂)₂pyrimidinyl, —(CH₂)₃imidazolyl, and the like, and derivatives thereof.

As used herein, the term “heteroaryl” refers to a monocyclic five to seven membered aromatic ring, or to a fused bicyclic aromatic ring system comprising two of such aromatic rings, which may be optionally substituted as herein further described, with multiple degrees of substitution being allowed. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzodioxolyl, benzothiophene, indole, indazole, benzimidizolyl, imidazopyridinyl, pyrazolopyridinyl, and pyrazolopyrimidinyl.

The term “heterocycle”, “heteroaliphatic” or “heterocyclyl” as used herein is intended to mean a 5-to 10-membered nonaromatic heterocycle containing from 1 to 4 heteroatoms selected from the group consisting of O, N and S, and includes bicyclic groups.

The term “heteromultimer” or “heteromultimeric” as used herein describes two or more polymers (e.g., polypeptides) that interact with each other by a non-peptidic, covalent bond (e.g., disulfide bond) and/or a non-covalent interaction (e.g., charge interactions, affinity interactions, metal coordination, physical adsorption, host-guest interactions, hydrophobic interactions, [symbol] stacking interactions, hydrogen bonding interactions, van der Waals interactions, magnetic interactions, electrostatic interactions, dipole-dipole interactions, etc.), wherein at least two of the polymers have a different sequence of monomer units from each other.

Reference herein to “immuno-interactive” includes reference to any interaction, reaction, or other form of association between molecules and in particular where one of the molecules is, or mimics, a component of the immune system.

The terms “linker group” and “linker” are used herein to mean a molecular entity that covalently links a first moiety and a second moiety to form a molecule comprising the first and second moiety. In some embodiments, the term “linker group” refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine.

As used herein, the term “moiety” refers to a component part or group present in a molecule. In specific embodiments, a moiety refers to a constituent of a repeated polymer structural unit. Exemplary moieties include acid or base species, sugars, carbohydrates, alkyl groups, aryl groups and any other molecular constituent useful in forming a polymer structural unit. The term “organic moiety” as used herein indicates a moiety that contains a carbon atom. In particular, organic groups include natural and synthetic compounds, and compounds including heteroatoms. Exemplary natural organic moieties include but are not limited to most sugars, some alkaloids and terpenoids, carbohydrates, lipids and fatty acids, nucleic acids, proteins, peptides and amino acids, vitamins and fats and oils. Synthetic organic groups refer to compounds that are prepared by reaction with other compounds.

The term “multi-specific molecule” as used herein refers to a molecule that binds to two or more different epitopes on one antigen or on two or more different antigens. In some embodiments, a multi-specific molecule is a multi-specific antibody or antibody like molecule. The term “multi-specific” includes “bi-specific.”

The term “nanoparticle” refers to a structure having at least one region with a dimension (e.g., length, width, diameter, etc.) of less than about 1,000 nm. In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is less than about 10 nm.

As used herein, the term “oxyalkylene” refers to a divalent group that is an oxy group bonded directly to an alkylene group. As used herein, the term “poly(oxyalkylene)” refers to a divalent group having multiple oxyalkylene groups. Suitable oxyalkylene groups typically have 1 to 100 carbon atoms.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In specific embodiments, the subject is a primate such as a human.

As used herein, the term “pendant” means attached to the polymer chain backbone as a side group, but not within the polymer chain backbone. The term “pendant” also includes attachment of such a group at a terminus of a polymer chain.

The terms “polymer” and “polymer chain” refer to macromolecules comprising repeating structural units (constitutional or monomeric units), e.g., from 5 up to one million or more monomeric units, connected by covalent chemical bonds. Polymer chains may be synthetic, semi-synthetic or naturally occurring, and comprise homopolymers (i.e., comprising the same repeating monomer unit) or copolymers (i.e., comprising at least two different monomer units). Copolymers can be periodic copolymers (e.g., where monomer residues A and B are arranged in a repeating sequence such as A-B-A-B-B-A-A-A-A-B-B-B), or random (or statistical) copolymers having random sequences of monomers A and B. Block copolymers typically comprise two or more homopolymer subunits linked to each other by covalent bond or a junction block. Block copolymers with two or three distinct blocks are called di-block copolymers (AAAAA-BBBBB) and tri-block copolymers (AAAAA-BBBBB-AAAAA), respectively. Polymer chains can be linear (with a single main chain) or branched (with one or more lateral chains attached to the main chain). The chain of the polymer containing the repeating units is often identified as the “polymer backbone”, while the units disposed at respective terminal ends (e.g., the α- and ω-ends) of the chain are generally identified as “terminal groups”.

“Self-assembly” refers to a process of spontaneous assembly of a higher order structure that relies on the natural attraction of the components of the higher order structure (e.g., molecules) for each other. It typically occurs through random movements of the molecules and formation of bonds based on size, shape, composition, or chemical properties.

The term “specificity”, as used herein, refers to the ability of an affinity moiety to bind preferentially to one antigen, versus a different antigen, and does not necessarily imply high affinity (as defined herein). An affinity moiety that can specifically bind to and/or that has affinity for a specific antigen or epitope thereof is said to be “against” or “directed against” the antigen or epitope. An affinity moiety is said to be “cross-reactive” for two different antigens or epitopes if it is specific for both these different antigens or antigenic determinants.

The terms “solid support”, “support structure”, and “substrate” as used herein are used interchangeably and refer to a material or group of materials having a rigid or semi-rigid surface or surfaces. There is no limitation to the shape or size of the support structures.

As used herein, the terms “specifically binds”, “specific binding” and the like refer to a molecule or moiety that binds with a target molecule or moiety with at least 2-fold greater affinity, as compared to a non-targeted molecule or moiety. In certain embodiments, a molecule or moiety specifically binds with a target molecule or moiety with at least 3-fold, 4-fold, 5-fold, 6-fold, 7-fold, 8-fold, 9-fold, 10-fold, 25-fold, 50-fold, 100-fold, 500-fold, 1000-fold, 5000-fold, 10000-fold, or greater affinity, as compared to a non-targeted molecule or moiety.

As used herein the term “target site” refers to a binding partner of a targeting ligand. The binding partner may be a molecule or macromolecule of a cell, a soluble molecule or a soluble macromolecule.

The term “targeting ligand” as used in the present disclosure indicates any molecule that can be presented on a polymer chain or on an assembly comprising a plurality of polymer chains for the purpose of interacting or engaging a specific target site, including specific cellular recognition, for example by enabling cell receptor attachment of the polymer chain or assembly.

Each embodiment described herein is to be applied mutatis mutandis to each and every embodiment unless specifically stated otherwise.
2. Abbreviations

The following abbreviations are used throughout the application:

aa=amino acid(s)

kDa=kilodalton(s)

Mw=molecular weight

PEG=polyethylene glycol

mPEG=methoxy polyethylene glycol

HBP=hyperbranched mPEG

s=second(s)

ms=millisecond(s)

min=minute(s)

h=hour(s)

wk=week(s)

d=day(s)
3. Targeting Constructs with Enhanced Avidity for a Target Site

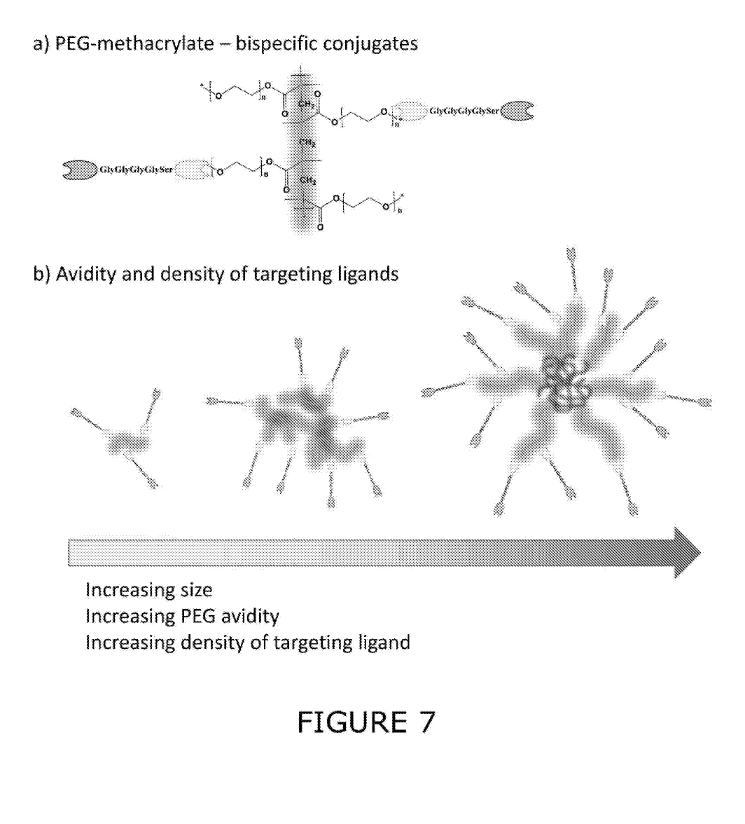

The present invention provides targeting constructs that are useful inter alia for delivery of payloads to a target site. The constructs take advantage of multiple affinity moiety-binding partners on a polymer chain to bind a plurality of multi-specific molecules each of which comprises an affinity moiety that binds with an affinity moiety-binding partner on the polymer chain and a targeting ligand that targets the targeting construct to a target site of interest. An advantageous feature of the construct design is that multiple (i.e., two or more) targeting ligands can be attached to a single polymer chain (which can be linear, branched or in an assembly). This enables decoration of the polymer chain with a higher density of targeting ligands that increase avidity of the polymer chain, even when assembled into polymeric vehicles or assemblies (e.g., particles including nanoparticles and microparticles), to the target site. A non-limiting example of a targeting construct showing polymer chains so decorated is shown schematically in FIG. 7.

Another advantageous feature of the construct design is the use of an affinity moiety for attaching a targeting ligand to the polymer chain. This permits facile binding of the polymer chain to the targeting ligand in a single step to confer specificity of the polymer chain to the target site. Accordingly, a plurality of multi-specific molecules can be attached to the polymer chain without the need to chemically modify the polymer chain, which facilitates rapid conversion of a non-targeted polymer chain to a targeted polymer chain with enhanced avidity for the target site.

The targeting constructs of the present invention are suitably represented by formula (I):

p-[α-L-λ]ₙ (I)

wherein:

p represents a polymer chain;

α-L-λ, independently for each occurrence, represents a multi-specific

molecule,

wherein:

α, independently for each occurrence, represents an affinity moiety that binds with the polymer chain;

L, independently for each occurrence, is absent or represents a linker group; and

λ, independently for each occurrence, represents a targeting ligand; and

n represents an integer of at least 2,

wherein the polymer chain comprises a plurality of affinity moiety-binding partners, and wherein individual affinity moiety-binding partners bind with the affinity moiety of a respective multi-specific molecule.
3.1 Polymer Chains

In accordance with the present invention, an individual polymer chain of a targeting construct comprises a plurality of binding partners for respective affinity moieties of two or more multi-specific molecules. The affinity moiety-binding partners may be the same or different. An individual affinity moiety-binding partner suitably comprises or is formed by one or more groups of at least one monomer residue of the polymer chain. In some embodiments, a first affinity moiety-binding partner comprises or is formed by one or more groups of at least one first monomer residue of the polymer chain and wherein a second affinity moiety-binding partner comprises or is formed by one or more groups of at least one second monomer residue of the polymer chain. In illustrative examples of this type, the first monomer residue and the second monomer residue are different. In other illustrative examples, the first monomer residue and the second monomer residue are the same, wherein the one or more groups of the first monomer residue are different to the one or more groups of the second monomer residue. In some embodiments, the first affinity moiety-binding partner binds with a first affinity moiety of the construct and the second affinity moiety-binding partner binds with a second affinity moiety of the construct, whereby the first affinity moiety and the second affinity moiety are different. An individual affinity moiety-binding partner may comprise an end group of the polymer chain and/or a pendant group of the polymer chain. Thus, the present invention contemplates embodiments in which the affinity moieties of the multi-specific molecules of a targeting construct bind with identical affinity moiety-binding partners along the backbone of the polymer chain. Alternatively, the affinity moieties of the multi-specific molecules of a targeting construct bind with different affinity moiety-binding partners and in these embodiments, one affinity moiety may bind with an affinity moiety-binding partner on the backbone of the polymer chain and another may bind to either a different affinity moiety-binding partner on the backbone of the polymer chain or on the terminus of the polymer chain. In preferred embodiments, an individual affinity moiety-binding partner on the polymer chains is an epitope to which an antigen-binding molecule binds.

Thus, the polymer chain (p) may comprises a plurality of repeat units [A-B] and is generally represented by formula (V):

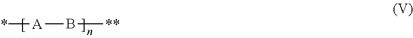

wherein:

A is an organic moiety linking a first B moiety of a first monomeric unit to a second B moiety of a second monomeric unit;

B is an organic moiety to which a side chain is optionally attached;

* and ** are terminal groups of the polymer chain; and

n is an integer from 1 to 100000, suitably 1 to 10000, 1 to 1000 or 1 to 100.

In illustrative example of this type, one or both of the terminal groups forms or comprises an affinity moiety-binding partner for binding with an affinity moiety of a multi-specific molecule. Representative groups of this type include but are not limited to alkoxy (e.g., methoxy, epoxy), arylene (e.g., phenylene), alkenylene (e.g., vinylene), oxyalkylene (e.g., oxyethylene), heterocyclic (e.g., pyrrole, pyrrolidone) and acrylic groups (e.g., acrylamide), or combination thereof.

Non-limiting examples of a polymer chain according to formula (V) are represented by formula (Va):

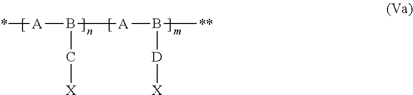

wherein:

A is an organic moiety linking a first B moiety of a first monomeric unit to a second B moiety of a second monomeric unit;

B is an organic moiety;

C and D represent structurally different side chains;

X is an affinity moiety-binding partner for binding with an affinity moiety;

* and ** are terminal groups of the polymer chain, which independently optionally form or comprise an affinity moiety-binding partner that is the same as or different to X;

n is an integer from 1 to 100000, suitably 1 to 10000, 1 to 1000 or 1 to 100; and

m is an integer from 1 to 100000, suitably 1 to 10000, 1 to 1000 or 1 to 100.

C and D may comprise different monomer residues or may comprise the same but different number of monomer residues. In these later embodiments, the different side chains are generally functionalized with the same functional group representing X. In specific embodiments, the functional group is selected from a methoxy group such and oxyethylene group.

Thus, when X of each monomeric residue is bound by a cognate affinity moiety of an respective multi-specific molecule, the polymer chain (p) is generally represented by formula (Vai):

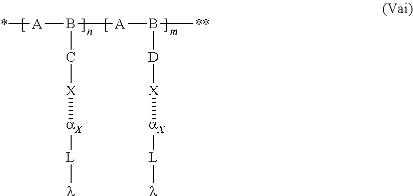

wherein:

A, B, C, D, X, n and m are the same as for formula (Va);

L, independently for each occurrence, is absent or represents a linker group; and

λ, independently for each occurrence, represents the same or different targeting ligand; and

αₓ is an affinity moiety that binds with X.

Other non-limiting examples of a polymer chain according to formula (V) are represented by formula (Vb):

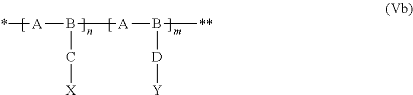

wherein:

A is an organic moiety linking a first B moiety of a first monomeric unit to a second B moiety of a second monomeric unit;

B is an organic moiety;

C and D are each side chains comprising at least one monomeric unit, wherein C and D are different;

X and Y represent distinct affinity moiety-binding partners for binding with different affinity moieties;

* and ** are terminal groups of the polymer chain, which independently optionally form or comprise an affinity moiety-binding partner that is the same as or different to X or Y;

n is an integer from 1 to 100000, suitably 1 to 10000, 1 to 1000 or 1 to 100; and

m is an integer from 1 to 100000, suitably 1 to 10000, 1 to 1000 or 1 to 100.

Like formula (Va), C and D of formula (Vb) may comprise different monomer residues or may comprise the same but different number of monomer residues. In either case, the different side chains are functionalized with different functional groups representing X and Y respectively. In specific embodiments, the different functional groups include a methoxy group such and oxyethylene group.

Accordingly, when X and Y are each bound my a cognate affinity moiety of an individual multi-specific molecule, the polymer chain (p) is generally represented by formula (Vbi):

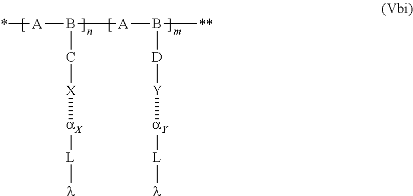

wherein:

A, B, C, D, X, Y, n and m are the same as for formula (Vb);

L, independently for each occurrence, is absent or represents a linker group; and

λ, independently for each occurrence of X and Y, represents the same or different targeting ligand; and

αₓ is an affinity moiety that binds with X; and

α_{y} is an affinity moiety that binds with Y.

Generally, the average molecular weight of the polymer chain will range from about 3 kDa to about 1000 kDa, from about 5 to about 500 kDa, from about 10 to about 1000 kDa, from about 20 to about 500 kDa, from about 5 kDa to about 600 kDa, from about 5 kDa to about 400 kDa, from about 3 to about 300 kDa, from about 5 kDa to about 250 kDa, from about 5 kDa to about 150 kDa, or from about 5 kDa to about 100 kDa. In certain embodiments, the polymer chain is about 5 kDa, about 1000 kDa, about 15 kDa, about 200 kDa, about 25 kDa, is about 300 kDa, about 35 kDa, about 400 kDa, about 45 kDa, about 500 kDa, about 55 kDa, about 600 kDa, about 65 kDa, about 700 kDa, about 75 kDa, about 800 kDa, about 850 kDa, about 90 kDa, about 95 kDa, about 100 kDa, about 110 kDa, about 120 kDa, about 130 kDa, about 140 kDa, about 150 kDa, about 160 kDa, about 170 kDa, about 180 kDa, about 190 kDa, about 200 kDa, about 300 kDa, about 350 kDa, about 400 kDa, about 450 kDa, about 500 kDa, about 550 kDa, about 600 kDa, about 650 kDa, about 700 kDa, about 700 kDa, about 800 kDa, about 850 kDa, about 900 kDa, about 950 kDa or about 1000 kDa.

Thus, polymer chains of the present invention are adapted to facilitate binding of at least two multi-specific molecules. For example, such polymer chains can comprise an end functional group (e.g., one or both of the α-end and the ω-end of the polymer chain) that comprises or forms an affinity moiety-binding partner for binding with an affinity moiety of a multi-specific molecule. Alternatively, or in addition, such polymer chains can comprise one or more monomeric residues having a pendant functional group that comprises or forms an affinity moiety-binding partner for binding with an affinity moiety of a multi-specific molecule. Alternatively, or in addition, such polymer chains can comprise one or more monomeric residues having two or more pendant functional groups—suitable for crosslinking between polymer chains. Such crosslinking monomeric residues can be a constituent moiety of a cross-linked polymer chain, as derived directly from a polymerization reaction that includes one or more polymerizable monomers comprising a multi-functional (e.g., bis-functional) crosslinking monomer.

Generally, the polymer chain can be a homopolymer (derived from polymerization of one single type of monomer —having essentially the same chemical composition) or a copolymer (derived from polymerization of two or more different monomers—having different chemical compositions). Polymer chains that are copolymers include random copolymers or block copolymers (e.g., diblock copolymer, triblock copolymer, higher-ordered block copolymer, etc.). Any given block copolymer can be conventionally configured and effected according to methods known in the art. The present invention also contemplates statistical copolymers, random copolymers, alternating copolymers, periodic copolymers, radial copolymers, graft copolymers, and combination thereof.

The polymer chain can be a linear polymer, or a non-linear polymer. Non-linear polymers can have various architectures, including for example branched polymers, brush polymers, star polymers, comb polymers, dendrimer polymers, and can be network polymers, cross-linked polymers, semi-cross-linked polymers, graft polymers, and combinations thereof. In certain non-limiting embodiments, non-linear polymers comprise pendant cognate binding partners, individual ones of which bind with an affinity moiety of the targeting construct.

Polymer chains of the present invention may be prepared by methods including Atom Transfer Radical Polymerization (ATRP), nitroxide-mediated living free radical polymerization (NMP), ring-opening polymerization (ROP), degenerative transfer (DT), or Reversible Addition Fragmentation Transfer (RAFT). In specific embodiments, a polymer can be a prepared by controlled (living) radical polymerization, such as reversible addition-fragmentation chain transfer (RAFT) polymerization. Such methods and approaches are generally known in the art, and are further described herein. Alternatively, a polymer can be a prepared by conventional polymerization approaches, including conventional radical polymerization approaches.

Generally, polymer chains prepared by controlled (living) radical polymerization, such as reversible addition-fragmentation chain transfer (RAFT) polymerization, may include moieties other than the monomeric residues (repeat units). For example, and without limitation, such polymer chains may include polymerization-process-dependent moieties at the α-end or at the ω-end of the polymer chain. Typically, for example, a polymer chain derived from controlled radical polymerization such as RAFT polymerization may further comprise a radical source residue covalently coupled with the α-end thereof. For example, the radical source residue can be an initiator residue, or the radical source residue can be a leaving group of a reversible addition-fragmentation chain transfer (RAFT) agent. Typically, as another example, a polymer chain derived from controlled radical polymerization such as RAFT polymerization may further comprise a chain transfer residue covalently coupled with the ω-end thereof. For example, a chain transfer residue can be a thiocarbonylthio moiety having a formula —SC(═S)Z, where Z is an activating group. Typical RAFT chain transfer residues are derived from radical polymerization in the presence of a chain transfer agent selected from xanthates, dithiocarbamates, dithioesters, and trithiocarbonates. The process-related moieties at the α-end or at the ω-end of the polymer chain or between blocks of different polymer chains can comprise or can be derivatized to comprise functional groups, e.g., that comprise or form at least a portion of a cognate binding partner of an affinity moiety, etc.

Generally, the polymer chains of the present invention include, by way of non-limiting examples, polyamides, proteins, polyesters, polystyrene, polyethers, polyketones, polysulfones, polyurethanes, polysiloxanes, polysilanes, chitosan, cellulose, amylase, polyacetals, polyethylene, glycols, poly(acrylate)s, poly(methacrylate)s, poly(vinyl alcohol), poly(vinyl pyrrolidone), poly(vinylidene chloride), poly(vinyl acetate), poly(alkylene glycol)s such as poly(ethylene glycol) and poly(propylene glycol), polystyrene, polyisoprene, polyisobutylenes, poly(vinyl chloride), poly(propylene), poly(lactic acid), polyisocyanates, polycarbonates, alkyds, phenolics, epoxy resins, polysulf[iota]des, polyimides, liquid crystal polymers, heterocyclic polymers, polypeptides, polyacetylene, polyquinoline, polyaniline, polypyrrole, polythiophene, poly(p-phenylene), fluoropolymers, or combinations thereof. In some embodiments, the backbone of the polymer chain is not a peptidic polymer. Preferred polymer chains comprise water-dispersible and in particular water soluble polymers. For example, suitable polymers include, but are not limited to, polysaccharides, polyesters, polyamides, polyethers, polycarbonates, polyacrylates, etc. Suitably, the polymer chains are biodegradable and/or biocompatible.

Accordingly, the various polymer chains included as constituent moieties of the targeting constructs of the present invention can comprise one or more repeat units—monomer (or monomeric) residues—derived from a process that includes polymerization. Such monomeric residues can optionally also include structural moieties (or species) derived from post-polymerization (e.g., derivatization) reactions. Monomeric residues are constituent moieties of the polymers chains, and accordingly, can be considered as constitutional units of the polymers. Generally, a polymer chain of the invention can comprise constitutional units that are derived (directly or indirectly via additional processes) from one or more polymerizable monomers.

Generally, any monomer suitable for providing the polymer chains described herein may be used to effect the invention. The monomers may be hydrophilic, hydrophobic, amphiphilic, anionic, cationic, neutral or zwitterionic in nature. In some embodiments, monomers suitable for use in the preparation of polymer chains provided herein include, by way of non-limiting example, one or more of the following monomers: butadienes, styrenes, propene, acrylates, methacrylates, vinyl ketones, vinyl esters, vinyl acetates, vinyl chlorides, vinyl fluorides, vinyl ethers, vinyl pyrrolidone, acrylonitrile, methacrylonitrile, acrylamide, methacrylamide allyl acetates, fumarates, maleates, ethylenes, propylenes, tetrafluoroethylene, ethers, isobutylene, fumaronitrile, vinyl alcohols, acrylic acids, amides, carbohydrates, esters, urethanes, siloxanes, formaldehyde, phenol, urea, melamine, isoprene, isocyanates, epoxides, bisphenol A, chlorsianes, dihalides, dienes, alkyl olefins, ketones, aldehydes, vinylidene chloride, anhydrides, saccharide, acetylenes, naphthalenes, pyridines, lactams, lactones, acetals, thiiranes, episulf[iota]de, peptides, or combinations thereof.

In various embodiments, the polymer chains can comprise one or more of the following monomer residues: methyl methacrylate, ethyl acrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, alpha-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, acrylates and styrenes selected from glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N-isopropyl (meth) acrylamide, N,N-dimethylacrylamide, N-tert-butylmethacrylamide, N-n-butylmethacrylamide, N-methylolacrylamide, N-ethylolacrylamide, vinyl benzoic acid (all isomers), diethylaminostyrene (all isomers), alpha-methylvinyl benzoic acid (all isomers), diethylamino alpha-methylstyrene (all isomers), p-vinylbenzenesulfonic acid, p-vinylbenzene sulfonic sodium salt, trimethoxysilylpropyl methacrylate, triethoxysilylpropyl methacrylate, tributoxysilylpropyl methacrylate, dimethoxymethylsilylpropyl methacrylate, diethoxymethylsilylpropylmethacrylate, dibutoxymethylsilylpropyl methacrylate, diisopropoxymethylsilylpropyl methacrylate, dimethoxysilylpropyl methacrylate, diethoxysilylpropyl methacrylate, dibutoxysilylpropyl methacrylate, diisopropoxysillpropyl methacrylate, trimethoxysilylpropyl acrylate, triethoxysilylpropyl acrylate, tributoxysilylpropyl acrylate, dimethoxymethylsilylpropyl acrylate, diethoxymethylsilylpropyl acrylate, dibutoxymethylsilylpropyl acrylate, diisopropoxymethylsilylpropyl acrylate, dimethoxysilylpropyl acrylate, diethoxysilylpropyl acrylate, dibutoxysilylpropyl acrylate, diisopropoxysilylpropyl acrylate, vinyl acetate, vinyl butyrate, vinyl benzoate, vinyl chloride, vinyl fluoride, vinyl bromide, maleic anhydride, N-arylmaleimide, N-phenylmaleimide, N-alkylmaleimide, N-butylimaleimide, N-vinylpyrrolidone, N-vinylcarbazole, butadiene, isoprene, chloroprene, ethylene, propylene, 1,5-hexadienes, 1,4-hexadienes, 1,3-butadienes, 1,4-pentadienes, vinylalcohol, vinylamine, N-alkylvinylamine, allylamine, N-alkylallylamine, diallylamine, N-alkyldiallylamine, alkylenimine, acrylic acids, alkylacrylates, acrylamides, methacrylic acids, alkylmethacrylates, methacrylamides, N-alkylacrylamides, N-alkylmethacrylamides, styrene, vinylnaphthalene, vinyl pyridine, ethylvinylbenzene, aminostyrene, vinylimidazole, vinylpyridine, vinylbiphenyl, vinylanisole, vinylimidazolyl, vinylpyridinyl, vinylpolyethyleneglycol, dimethylaminomethylstyrene, trimethylammonium ethyl methacrylate, trimethylammonium ethyl acrylate, dimethylamino propylacrylamide, trimethylammonium ethylacrylate, trimethylanunonium ethyl methacrylate, trimethylammonium propyl acrylamide, dodecyl acrylate, octadecyl acrylate, or octadecyl methacrylate monomers, or combinations thereof. Suitably, individual monomer residues of the polymer chain are hydrophilic. In representative examples of this type, the polymer chain is a hydrophile.

In specific embodiments, the polymer chains comprise monomer residues selected from sec-butyl acrylate, n-butyl acrylate, t-butyl acrylate, t-butyl methacrylate, methylmethacrylate, N-dimethyl-aminoethyl(methyl)acrylate, N,N-dimethylaminopropyl-(meth)acrylate, t-butylaminoethyl (methyl)acrylate, N,N-diethylaminoacrylate, acrylate terminated poly(ethylene oxide), methacrylate terminated poly(ethylene oxide), methoxy poly(ethylene oxide) methacrylate, butoxy poly(ethylene oxide) methacrylate, acrylate terminated poly(ethylene glycol), methacrylate terminated poly(ethylene glycol), methoxy poly(ethylene glycol) methacrylate, butoxy poly(ethylene glycol) methacrylate, or combinations thereof. In specific embodiments, the polymer chain comprises monomer residues selected from poly(alkylene glycol)(meth)acrylate. In illustrative examples of these embodiments, the monomer residue comprises 1 to 100 alkylene oxide units.

In some embodiments, polymer chains can include repeat units derived from functionalized monomers, including versions of the aforementioned monomers. A functionalized monomer, as used herein, can include a monomer comprising a masked (protected) or non-masked (unprotected) functional group, e.g., a group to which other moieties can be covalently attached following the polymerization. The non-limiting examples of such groups are primary amino groups, carboxyls, thiols, hydroxyls, azides, and cyano groups. Several suitable masking groups are available (see, e.g., T. W. Greene & P. G. M. Wuts, Protective Groups in Organic Synthesis (2nd edition) J. Wiley & Sons, 1991. P. J. Kocienski, Protecting Groups, Georg Thieme Verlag, 1994).

The polymer chains of the present invention include unimer or monoblock polymers, which are generally synthetic products of a single polymerization step. The term monoblock polymer includes a copolymer such as a random copolymer (i.e., a product of polymerization of more than one type of monomers) and a homopolymer (i.e., a product of polymerization of a single type of monomers).

In some embodiments, the polymer chain is block copolymer such as but not limited to a diblock copolymer, a tri-block copolymer or a higher-ordered block copolymer. For example, a diblock copolymer can comprise two blocks; a schematic generalization of such a polymer is represented by the following: [Aₐ/B_{b}/C_{c}/ . . . ]ₘ-[Xₓ/Y_{y}/Z_{z}/ . . . ]ₙ, wherein each letter stands for a constitutional or monomeric unit, and wherein each subscript to a constitutional unit represents the mole fraction of that unit in the particular block, the three dots indicate that there may be more (there may also be fewer) constitutional units in each block and m and n indicate the molecular weight (or weight fraction) of each block in the diblock copolymer. As suggested by such schematic representation, in some instances, the number and the nature of each constitutional unit is separately controlled for each block. In some embodiments, individual constitutional or monomeric units or combinations of such units may form or comprise an affinity moiety-binding partner for binding with an affinity moiety of a respective multi-specific molecule.

The above schematic is not meant to, and should not be construed to, infer any relationship whatsoever between the number of constitutional units or between the number of different types of constitutional units in each of the blocks. Nor is the schematic meant to describe any particular number or arrangement of the constitutional units within a particular block. In each block the constitutional units may be disposed in a purely random, an alternating random, a regular alternating, a regular block or a random block configuration unless expressly stated to be otherwise. A purely random configuration, for example, may have the form: x-x-y-z-x-y-y-z-y-z-z-z . . . . An exemplary alternating random configuration may have the form: x-y-x-z-y-x-y-z-y-x-z . . . , and an exemplary regular alternating configuration may have the form: x-y-z-x-y-z-x-y-z . . . . An exemplary regular block configuration may have the following general configuration: . . . x-x-x-y-y-y-z-z-z-x-x-x . . . , while an exemplary random block configuration may have the general configuration: . . . x-x-x-z-z-x-x-y-y-y-y-z-z-z-x-x-z-z-z- . . . . In a gradient polymer, the content of one or more monomeric units increases or decreases in a gradient manner from the α-end of the polymer to the ω-end.

In specific embodiments, the polymer chain comprises poly(ethylene glycol)methacrylate repeating units of formula (VI):

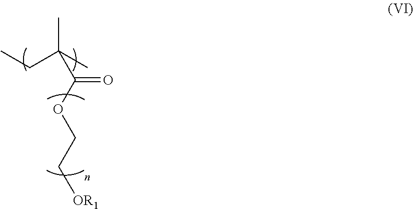

wherein:

n is an integer from 1 to 100, suitably from 5 to 50, more suitably from 10 to 30; and

R₁ is H or C₁-C₆ alkyl (e.g., methyl).

The polymer chain may be soluble or immobilized. In embodiments in which it is immobilized, the polymer chain is suitably in the form of, or contained in, or tethered to, a solid support or substrate. The solid support or substrate can be a well, a multi-well plate, a dipstick, a resin, a gel, a tube, a particle, a strip, a chip, an electrode, a sensor, a biosensor, a membrane, a sheet, a cone, a chamber, or a dish. The solid support can be any suitable geometric configuration (e.g., planar or non-planar). In some embodiments, the solid support is selected from plastic surfaces, latex, dextran, polystyrene surfaces, polypropylene surfaces, polyacrylamide gels, polymeric beads and silicon wafers. In some embodiments, the solid support is a plastics surface (e.g., a planar surface of a multi-well plate or flow cell channel). These embodiments are advantageous inter alia for facile preparation of analytical tools for detecting an analyte of interest, illustrative examples of which include cells, viruses, proteins, hormones, antibodies, antigens, haptens, lectins, receptors, ligands, oligonucleotides, peptides, or any other chemical or biological compound or composition. In certain embodiments, an immobilized polymer chain (e.g., in a well or on a solid particle) is contacted with a plurality of multi-specific molecules that have binding specificity with the polymer chain and with a chosen analyte, to thereby prepare an immobilized targeting construct for detecting the analyte. In illustrative examples of this type, a range of targeting constructs can be prepared as analytic tools with specificity for different targets analytes simply by using the same affinity moiety for binding with a polymer chain of a support structure (e.g., a multi-well plate, biochip or microparticle), and different affinity moieties with specificity to different target analytes.
3.2 Multi-specific Molecules

Multi-specific molecules of the present invention comprise an affinity moiety with specificity for a cognate binding partner on a polymer chain and a targeting ligand with specificity for a target site. An optional linker may be provided to space the affinity moiety from the targeting ligand. Thus, a multi-specific molecule may be represented by the following formula:

α-L-80 (III)

wherein:

α, independently for each occurrence, represents an affinity moiety that binds with the polymer chain;

L, independently for each occurrence, is absent or represents a linker group; and

λ, independently for each occurrence, represents a targeting ligand that targets the construct to the target site.
3.2.1 Affinity Moieties

The affinity moiety includes and encompasses any molecule or moiety that binds with a group or groups on an individual polymer chain. The affinity moiety is suitable selected from antigen-binding molecules, illustrative examples of which include antibodies and non-antibody targeting molecules.

In some embodiments, the affinity moiety is an antigen-binding molecule such as, but not limited to, an antibody, antigen-binding antibody fragment, or a non-antibody targeting molecule that binds specifically to an affinity moiety-binding partner of the polymer chain. The affinity moiety may also encompass protein scaffolds whereby peptides with affinity for an antigen are embedded within the protein scaffold in a manner that allows the peptide(s) to be displayed and contact an epitope.

Antibodies contemplated by the present invention include whole antibodies and antigen-binding antibody fragments. Thus, antibodies may be selected from naturally occurring antibodies that comprise at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as VL) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen or epitope thereof. The constant regions of the antibodies may mediate the binding of the immunoglobulin to host tissues or factors, including various cells of the immune system (e.g., effector cells) and the first component (Clq) of the classical complement system. Non-limiting examples of antibodies include monoclonal antibodies, human antibodies, humanized antibodies, camelized antibodies, chimeric antibodies, bi-specific or multiple-specific antibody and anti-idiotypic (anti-Id) antibodies. The antibodies can be of any isotype (e.g., IgG, IgE, IgM, IgD, IgA and IgY), class (e.g., IgG1, IgG2, IgG3, IgG4, IgA1 and IgA2) or subclass.

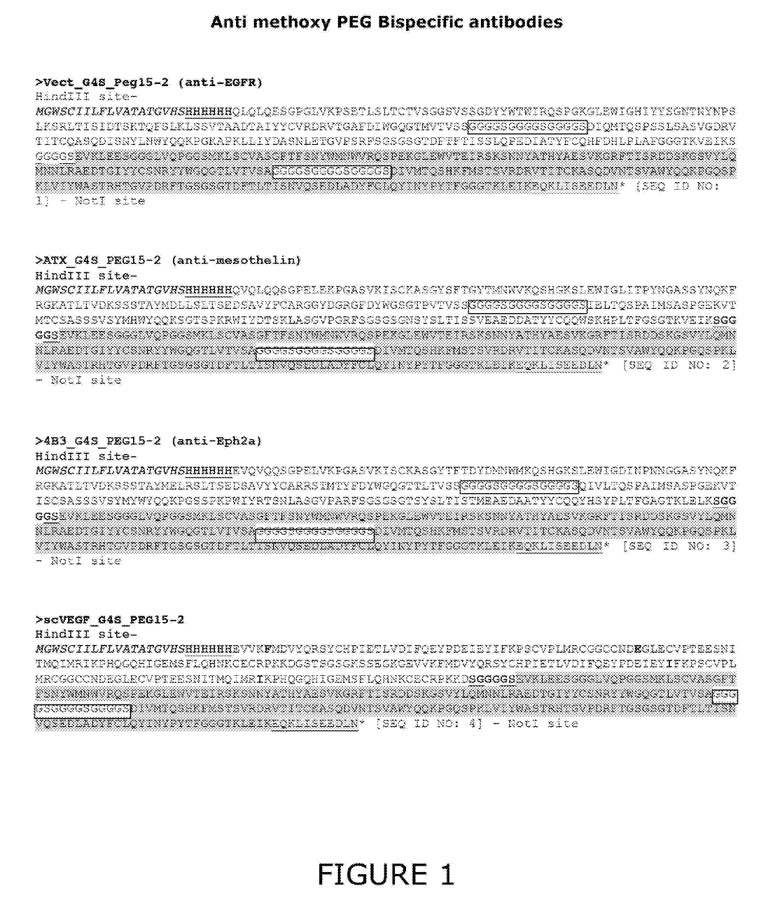

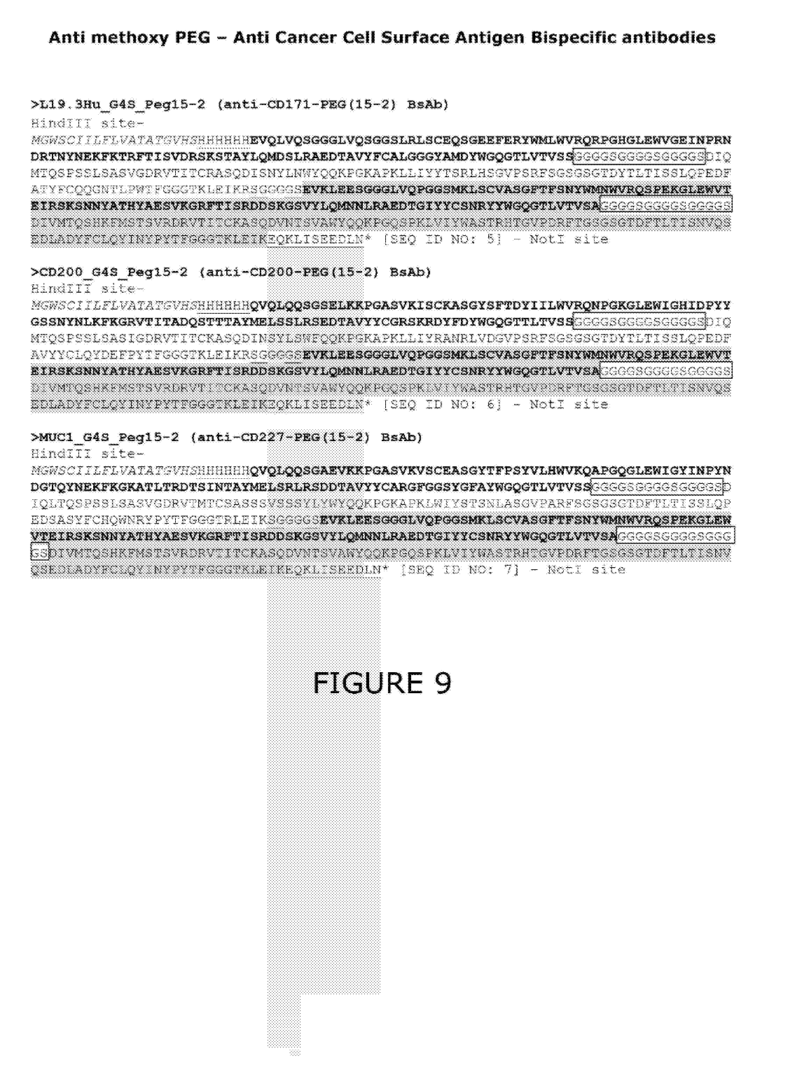

Generally, antibody fragments include portions of an antibody. In some embodiments, these portions are part of the contact domain(s) of an antibody. In some other embodiments, these portion(s) are antigen-binding fragments that retain the ability to specifically bind with an epitope. Examples of binding fragments include, but are not limited to, single-chain Fv (scFv), Fab fragments, monovalent fragments consisting of the V_{L}, V_{H}, C_{L} and C_{H1} domains; a F(ab)₂ fragment, bivalent fragments comprising two Fab fragments linked together by a disulfide bridge at the hinge region; Fd fragments consisting of the VH and CH1 domains; a Fv fragment consisting of the V_{L} and V_{H} domains of a single arm of an antibody; dAb fragments (Ward et al., 1989. Nature 341:544-546), which consists of a V_{H} domain; and an isolated complementarity determining region (CDR). Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23: 1126-1136). Antibody fragments can be incorporated into single chain molecules comprising a pair of tandem Fv segments (V_{H}—C_{H1}—V_{H}-—C_{H1}) which, together with complementary light chain polypeptides, form a pair of antigen binding regions (as disclosed, e.g., Zapata et al. (1995. Protein Eng. 8:1057-1062); and U.S. Pat. No. 5,641,870). In specific embodiments, the affinity moiety is a scFv that binds with a PEG molecule (e.g., a mPEG molecule), an illustrative example of which is showin in the targeting constructs illustrated in FIGS. 1 and 9.

Nanobodies are also contemplated as affinity moieties of the present invention. Nanobodies are single-domain antibodies of about 12-15 kDa in size (about 110 amino acids in length) and can selectively bind to target antigens, like full-size antibodies, and have similar affinities for antigens. However, because of their much smaller size, they may be capable of better penetration into tissues. The smaller size also contributes to the stability of the nanobody, which is more resistant to pH and temperature extremes than full size antibodies (Van Der Linden et al., 1999. Biochim Biophys Acta 1431:37-46). Single-domain antibodies were originally developed following the discovery that camelids (camels, alpacas, llamas) possess fully functional antibodies without light chains (e.g., Hamsen et al., 2007. Appl Microbiol Biotechnol. 77:13-22). The heavy-chain antibodies consist of a single variable domain (Van) and two constant domains (C_{H2} and C_{H3}). Like antibodies, nanobodies may be developed and used as multivalent and/or bispecific constructs. The plasma half-life of nanobodies is shorter than that of full-size antibodies, with elimination primarily by the renal route. Because they lack an Fc region, they do not exhibit complement dependent cytotoxicity. Nanobodies may be produced by immunization of camels, llamas, alpacas or sharks with target antigens such as polymer chains, following by isolation of mRNA, cloning into libraries and screening for antigen binding. Nanobody sequences may be humanized by standard techniques (e.g., Jones et al., 1986. Nature 321:522, Riechmann et al., 1988. Nature 332:323, Verhoeyen et al., 1988. Science 239:1534, Carter et al., 1992. Proc Natl Acad Sci. USA 89:4285, Sandhu, 1992. Crit. Rev. Biotech. 12:437, Singer et al., 1993, J. Immun. 150:2844). Humanization is relatively straightforward because of the high homology between camelid and human FR sequences.

In specific embodiments, the affinity moiety is an antibody fragment that comprises the V_{H} and V_{L} domains of antibody, wherein these domains are present in a single polypeptide chain. Preferably, the Fv polypeptide further comprises a polypeptide linker between the V_{H} and V_{L} domains, which enables the scFv to form the desired structure for antigen binding. For a review of scFv see Pluckthun in The Pharmacology of Monoclonal Antibodies, vol. 113, Rosenburg and Moore eds., (1994) Springer-Verlag, New York, pp. 269-315.

Non-limiting examples of antibodies that bind with polymer epitopes include: monoclonal antibodies that bind with polyacrylate polymers as disclosed, for example, in EP 0 540 314; scFv molecules that bind with poly(vinylpyrrolidone) (PVP) as described, for example, by Soshee et al. (2014, Biomacromol. 15:113-121); antibodies that bind with mPEG and with the PEG backbone, as disclosed for example in U.S. Pat. Appl. Pub. No. 20120015380. These publications are incorporated by reference herein in their entirety.

Also included within the scope of the present invention are antibody fusion proteins in which an antibody or antibody fragment is linked to another protein or peptide. The fusion protein may comprise a single antibody component, a multivalent or multispecific combination of different antibody components or multiple copies of the same antibody component.

In certain embodiments, the affinity moieties described herein may comprise one or more avimer sequences. Avimers are a class of binding proteins somewhat similar to antibodies in their affinities and specificities for various target molecules. They were developed from human extracellular receptor domains by in vitro exon shuffling and phage display. (Silverman et al., 2005. Nat. Biotechnol. 23:1493-94; Silverman et al., 2006. Nat. Biotechnol. 24:220). The resulting multidomain proteins may comprise multiple independent binding domains that may exhibit improved affinity (in some cases sub-nanomolar) and specificity compared with single-epitope binding proteins. Additional details concerning methods of construction and use of avimers are disclosed, for example, in U.S. Pat. Appl. Pub. Nos. 20040175756, 20050048512, 20050053973, 20050089932 and 20050221384, the Examples section of each of which is incorporated herein by reference.

Certain embodiments of affinity moieties relate to binding peptides and/or peptide mimetics of various polymer groups. Binding peptides may be identified by any method known in the art, including but not limiting to the phage display technique. Various methods of phage display and techniques for producing diverse populations of peptides are well known in the art. For example, U.S. Pat. Nos. 5,223,409; 5,622,699 and 6,068,829 disclose methods for preparing a phage library. The phage display technique involves genetically manipulating bacteriophage so that small peptides can be expressed on their surface (Smith and Scott, 1985, Science 228:1315-1317; Smith and Scott, 1993, Meth. Enzymol. 21:228-257). In addition to peptides, larger protein domains such as single-chain antibodies may also be displayed on the surface of phage particles (Arap et al., 1998, Science 279:377-380). Non-limiting examples of polymer-binding peptides include: HWGMWSY, which is a polystyrene-binding peptide (see, e.g., Vodnik et al., 2012. Anal Biochem. 424:83-86); TLHPAAD, which is epoxy group-binding peptide (see, e.g., Swaminathan et al., 2013. Mater Sci Eng C Mater Biol Appl. 33(5):3082-3084); THRTSTLDYFVI, which is a polypyrrole-binding peptide (see, e.g., Nickels et al., 2013. J Biomed Mater Res A. 101(5):1464-1471); and HTDWRLGTWHHS, which is poly(phenylene vinylene)-binding peptide (see, e.g., Ejima et al., 2010. Langmuir 26(22):17278-17285). These publications are incorporated by reference herein in their entirety.

In certain embodiments, an affinity moiety may be an aptamer. Methods of constructing and determining the binding characteristics of aptamers are well known in the art. For example, such techniques are described in U.S. Pat. Nos. 5,582,981, 5,595,877 and 5,637,459, the Examples section of each incorporated herein by reference. Methods for preparation and screening of aptamers that bind to particular targets of interest are well known, for example U.S. Pat. No. 5,475,096 and U.S. Pat. No. 5,270,163, the Examples section of each incorporated herein by reference. Aptamers may be prepared by any known method, including synthetic, recombinant, and purification methods, and may be used alone or in combination with other ligands specific for the same target. In general, a minimum of approximately 3 nucleotides, preferably at least 5 nucleotides, are necessary to effect specific binding. Aptamers of sequences shorter than 10 bases may be feasible, although aptamers of 10, 20, 30 or 40 nucleotides may be preferred. Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR₂, P(O)R, P(O)OR′, CO, or CNR₂, wherein R is H or C₁-C₂₀ alkyl and R′ is C₁-C₂₀ alkyl; in addition, this group may be attached to adjacent nucleotides through O or S, Not all linkages in an oligomer need to be identical.

Certain alternative embodiments may utilize affibodies in place of antibodies. Affibodies are commercially available from Affibody AB (Solna, Sweden). Affibodies are small proteins that function as antibody mimetics and are of use in binding target molecules including affinity moiety-binding partners on the polymer chains. Affibodies were developed by combinatorial engineering on an alpha helical protein scaffold (Nord et al., 1995. Protein Eng. 8:601-8; Nord et al., 1997. Nat Biotechnol. 15:772-77). The affibody design is based on a three-helix bundle structure comprising the IgG binding domain of protein A (Nord et al., 1995; 1997). Affibodies with a wide range of binding affinities may be produced by randomization of thirteen amino acids involved in the Fc binding activity of the bacterial protein A (Nord et al., 1995; 1997). After randomization, the PCR amplified library was cloned into a phagemid vector for screening by phage display of the mutant proteins. The phage display library may be screened against any known antigen, including polymer chains and their moieties, using standard phage display screening techniques (e.g., Pasqualini and Ruoslahti, 1996. Nature 380:364-366; Pasqualini, 1999. Quart. J. Nucl. Med. 43:159-162), in order to identify one or more affibodies against a polymer chain or moiety.

Fynomers can also bind to target antigens with a similar affinity and specificity to antibodies. Fynomers are based on the human Fyn SH3 domain as a scaffold for assembly of binding molecules. The Fyn SH3 domain is a fully human, 63-aa protein that can be produced in bacteria with high yields. Fynomers may be linked together to yield a multispecific binding protein with affinities for two or more different antigen targets. Fynomers are commercially available from COVAGEN AG (Zurich, Switzerland).
3.2.2 Linker Groups

The linker group(s) may be an alkylene chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker group itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker groups can be biologically inactive, such as a PEG, polyglycolic acid, or polylactic acid chain, or can be biologically active, such as an oligo-or polypeptide that, when cleaved from the moieties, binds a receptor, deactivates an enzyme, etc. Various oligomeric linker groups that are biologically compatible and/or bioerodible are known in the art, and the selection of the linkage may influence the ultimate properties of the material, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc. In certain embodiments, the linker group represents at least one (e.g., 1, 2, 3, 4, 5, 6, 7, 8, 9, 10, or more) derivatized or non-derivatized amino acids. In illustrative examples of this type, the linker group may be selected from: [GGGGS]ₙ, [GGGGG]ₙ, [GGGKGGGG]ₙ, [GGGNGGGG]ₙ, [GGGCGGGG]ₙ, wherein n is an integer from 1 to 10, suitably 2 to 5, more suitably 3 to 4.
3.2.3 Targeting Ligands

The targeting ligand targets the targeting construct to, and generally has specificity for the target site, which is suitably a binding partner of the ligand. The binding partner may be a molecule or macromolecule of a cell, a soluble molecule or a soluble macromolecule. The targeting ligand may be synthetic, semi-synthetic, or naturally occurring. Materials or substances which may serve as targeting ligands include, for example, proteins, including antigen-binding molecules as described for example above, hormones, hormone analogues, glycoproteins and lectins, peptides, polypeptides, amino acids, sugars, saccharides, including monosaccharides and polysaccharides, carbohydrates, small molecules, vitamins, steroids, steroid analogs, hormones, cofactors, bioactive agents, and genetic material, including nucleosides, nucleotides, nucleotide acid constructs and polynucleotides.

The targeting ligand may be selected from affinity moieties (e.g., antibodies, antigen-binding antibody fragments, or non-antibody targeting molecules), as defined for example above, cytokines, chemokines, growth factors (e.g., granulocyte colony stimulating factor (G-CSF), granulocyte macrophage colony stimulating factor (GM-CSF), epidermal growth factor (EGF), fibroblast growth factor (FGF), keratinocyte growth factor (KGF)), interferons, erythropoietin (EPO), TNF-α, interleukins, integrins, immunoglobulins, hormones (e.g., insulin, gonadotropins, growth hormone) and hormone analogues, peptides, transferrin, proteins that interact with a cell surface molecule or with a pattern recognition receptor, tumor receptor binding molecules, and molecules involved in vascular lesions, amino acids, sugars, saccharides, including monosaccharides and polysaccharides, carbohydrates, glycoproteins, lectins, small molecules, including drugs, vitamins, steroids, steroid analogs, cofactors, bioactive agents, and genetic material, including nucleosides, nucleotides, nucleic acid constructs and polynucleotides. In specific embodiments, the targeting ligand is an scFv.

Ligand-mediated targeting to specific tissues through binding to their respective receptors on the cell surface offers an attractive approach to improve the tissue-specific delivery of payloads. Specific targeting to disease-relevant cell types and tissues may help to lower the effective dose, reduce side effects and consequently maximize the therapeutic index. Carbohydrates and carbohydrate clusters with multiple carbohydrate motifs represent an important class of targeting ligands, which allow the targeting of drugs to a wide variety of tissues and cell types. For examples, see Hashida, et al., 2001. Adv Drug Deliv Rev. 52:187-9; Monsigny et al., 1994. Adv Drug Deliv Rev. 14:1-24; Gabius et al., 1996. Eur J Pharm and Biopharm 42:250-261; Wadhwa and Rice, 1995. J Drug Target. 3:111-127. Carbohydrate based targeting ligands include, but are not limited to, D-galactose, multivalent galactose, N-acetyl-D-galactose (GalNAc), multivalent GalNAc, e.g. GalNAC2 and GalNAc3; D-mannose, multivalent mannose, multivalent lactose, N-acetyl-galactosamine, N-acetyl-glucosamine, multivalent fucose, glycosylated polyaminoacids and lectins. The term multivalent indicates that more than one monosaccharide unit is present. Such monosaccharide subunits may be linked to each other through glycosidic linkages or linked to a scaffold molecule.

Lipophilic moieties, such as cholesterol or fatty acids can substantially enhance plasma protein binding and consequently circulation half-life. In addition, binding to certain plasma proteins, such as lipoproteins, has been shown to increase uptake in specific tissues expressing the corresponding lipoprotein receptors (e.g., LDL-receptor or the scavenger receptor SR-B1). For examples, see Bijsterbosch et al., 2000. Nucleic Acids Res. 28:2717-25; Wolfrum et al., 2007). Nat Biotechnol. 25:1149-57. Exemplary lipophilic moieties that enhance plasma protein binding include, but are not limited to, sterols, cholesterol, fatty acids, cholic acid, lithocholic acid, dialkylglycerides, diacylglyceride, phospholipids, sphingolipids, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyDlithocholic acid, O3-(oleoyl)cholenic acid, dimethoxytrityl, phenoxazine, aspirin, naproxen, ibuprofen, vitamin E and biotin etc.

Folates represent another class of ligands, which has been widely used for targeted drug delivery via the folate receptor. This receptor is highly expressed on a wide variety of tumor cells, as well as other cells types, such as activated macrophages. For examples, see Matherly and Goldman, 2003. Vitamins Hormones 66:403-456; Sudimack and Lee, 2000. Adv Drug Delivery Rev. 41:147-162. Similar to carbohydrate-based ligands, folates have been shown to be capable of delivering a wide variety of drugs, including nucleic acids and even liposomal carriers. For examples, see Reddy et al., 1999. J Pharm Sci. 88:1112-1118; Lu and Low, 2002. Adv Drug Delivery Rev. 54:675-693.

The targeting ligands can also include other receptor binding ligands such as hormones and hormone receptor binding ligands. A targeting ligand can be a thyrotropin, melanotropin, lectin, glycoprotein, surfactant protein A, mucin, glycosylated polyaminoacids, transferrin, bisphosphonate, polyglutamate, polyaspartate, a lipid, folate, vitamin B12, biotin, or an aptamer.

The targeting ligands also include proteins, peptides and peptidomimetics that bind with a target site. A peptidomimetic is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long Such peptides include, but are not limited to, RGD containing peptides and peptidomimetics that can target cancer cells, in particular cells that exhibit αᵥβ₃ integrin. Targeting peptides can be linear or cyclic, and include D-amino acids, non-peptide or pseudo-peptide linkages, peptidyl mimics. In addition the peptide and peptide mimics can be modified, e.g., glycosylated or methylated. Synthetic mimics of targeting peptides are also included.

In specific embodiments, the targeting ligands bind with target binding partners selected from: carbonic anhydrase IX, CCCL19, CCCL21, CSAp, CD1, CD1a, CD2, CD3, CD4, CDS, CD8, CD11A, CD14, CD15, CD16, CD18, CD19, IGF-1R, CD20, CD21, CD22, CD23, CD25, CD29, CD30, CD32b, CD33, CD37, CD38, CD40, CD40L, CD45, CD46, CD47, CD52, CD54, CD55, CD59, CD64, CD66a-e, CD67, CD70, CD70L, CD72, CD74, CD79a, CD79b, CD80, CD83, CD95, CD126, CD133, CD138, CD147, CD154, CD171, CD200, AFP, PSMA, CEACAM5, CEACAM-6, c-MET, B7, ED-B of fibronectin, Factor H, FHL-1, Flt-3, folate receptor, GROB, histone H2B, histone H3, histone H4, HMGB-1, hypoxia inducible factor (HIF), HM1.24, insulin-like growth factor-1 (ILGF-1), IFNγ, IFN-α, IFN-α, IL-2, IL-4R, IL-6R, IL-13R, IL-15R, IL-17R, IL-18R, IL-6, IL-8, IL-12, IL-15, IL-17, IL-18, IL20Rα, IL-23, IL-25, IP-10, LIV-1, MAGE, mCRP, MCP-1, MIP-1A, MIP-1B, MIF, MUC1, MUC2, MUC3, MUC4, MUC5, MUC5a,c, MUC16, PAM4 antigen, NCA-95, NCA-90, Ia, HM1.24, EGP-1 (TROP-2), EGP-2, HLA-DR, tenascin, Le(γ), RANTES, T101, TAC, Tn antigen, Thomson-Friedenreich antigens, tumor necrosis antigens, TNF-α, TRAIL receptor (R1 and R2), VEGFR, EGFR, FGFR, P1GF, complement factors C3, C3a, C3b, C5a, C5, and an oncogene product, B7, Ia, Ii, HMI.24, HLA-DR (e.g., HLA-DR10), NCA95, NCA90, HCG and sub-units, CEA (CEACAM5), CEACAM-6, CSAp, EGP-I, EGP-2, Ba 733, KC4 antigen, KS-I antigen, KS1-4, Le-Y, PIGF, ED-B fibronectin, NCA 66a-d, PAM-4 antigen, PSA, PSMA, RSS, SIOO, TAG-72, TIOI, TAG TRAIL-RI, TRAIL-R2, p53, tenascin, insulin growth factor-1 (IGF-I), Tn antigen, bone morphogenetic protein receptor-type IB (BMPR1B), E16, six transmembrane epithelial antigen of prostate (STEAP1), megakaryocyte potentiating factor (MPF), type II sodium-dependent phosphate transporter 3b (Napi3b), Semaphorin 5b (Sema 5b), PSCA h1g, Endothelin type B receptor (ETBR), MSG783, six transmembrane epithelial antigen of prostate 2 (STEAP2), transient receptor potential cation channel subfamily M, member 4 (TrpM4), teratocarcinoma-derived growth factor (CRIPTO), Fc receptor-like protein 2

(FcRH2), HER2, Epidermal growth factor receptor (EGFR) Brevican, Ephb2R, ASLG659, PSCA, GEDA, B cell-activating factor receptor (BAFF-R), CXCRS, HLA-DOB, Purinergic receptor P2X ligand-gated ion channel 5 (P2X5), Lymphocyte antigen 64 (LY64), Fc receptor-like protein 1 (FcRH1), Immunoglobulin superfamily receptor translocation associated 2 (IRTA2), a matrix metalloproteinase, oxidized LDL, scavenger receptor A, CD36, CD68, lectin-like oxidized LDL receptor-1 (LOX-1), SR-A1 and SR-B1.

In specific embodiments, the target-binding partner is a cell surface antigen, which suitably undergoes internalization, such as a protein, sugar, lipid head group or other antigen on the cell surface. In representative examples of this type, a payload associated with the targeting construct modulates (e.g., interferes) with cellular processes or images the cell. In some embodiments, therefore, a targeting construct of the present invention binds with a cell surface antigen through its targeting ligand and the targeting construct is internalized into the cell. Suitably, the internalization is mediated by endocytosis. In some embodiments, binding of the targeting construct with the cell surface antigen detectably agonizes or antagonizes an activity of the cell surface antigen. In some embodiments, binding of the targeting construct with the cell surface antigen detectably agonizes or antagonizes an intracellular pathway. In some embodiments, binding of the targeting construct with the cell surface antigen inhibits proliferation, survival or viability of a cell with which the cell surface antigen is associated.

A large number of antibodies against various disease targets, including but not limited to tumor-associated antigens, have been deposited at various depository institutions including for example the American Type Culture Collection (ATCC, Manassas, Va.) ATCC and/or have published variable region sequences and are available for use in the preparation of targeting ligands. See, e.g., U.S. Pat. Nos. 7,312,318; 7,282,567; 7,151,164; 7,074,403; 7,060,802; 7,056,509; 7,049,060; 7,045,132; 7,041,803; 7,041,802; 7,041,293; 7,038,018; 7,037,498; 7,012,133; 7,001,598; 6,998,468; 6,994,976; 6,994,852; 6,989,241; 6,974,863; 6,965,018; 6,964,854; 6,962,981; 6,962,813; 6,956,107; 6,951,924; 6,949,244; 6,946,129; 6,943,020; 6,939,547; 6,921,645; 6,921,645; 6,921,533; 6,919,433; 6,919,078; 6,916,475; 6,905,681; 6,899,879; 6,893,625; 6,887,468; 6,887,466; 6,884,594; 6,881,405; 6,878,812; 6,875,580; 6,872,568; 6,867,006; 6,864,062; 6,861,511; 6,861,227; 6,861,226; 6,838,282; 6,835,549; 6,835,370; 6,824,780; 6,824,778; 6,812,206; 6,793,924; 6,783,758; 6,770,450; 6,767,711; 6,764,688; 6,764,681; 6,764,679; 6,743,898; 6,733,981; 6,730,307; 6,720,155; 6,716,966; 6,709,653; 6,693,176; 6,692,908; 6,689,607; 6,689,362; 6,689,355; 6,682,737; 6,682,736; 6,682,734; 6,673,344; 6,653,104; 6,652,852; 6,635,482; 6,630,144; 6,610,833; 6,610,294; 6,605,441; 6,605,279; 6,596,852; 6,592,868; 6,576,745; 6,572,856; 6,566,076; 6,562,618; 6,545,130; 6,544,749; 6,534,058; 6,528,625; 6,528,269; 6,521,227; 6,518,404; 6,511,665; 6,491,915; 6,488,930; 6,482,598; 6,482,408; 6,479,247; 6,468,531; 6,468,529; 6,465,173; 6,461,823; 6,458,356; 6,455,044; 6,455,040, 6,451,310; 6,444,206; 6,441,143; 6,432,404; 6,432,402; 6,419,928; 6,413,726; 6,406,694; 6,403,770; 6,403,091; 6,395,276; 6,395,274; 6,387,350; 6,383,759; 6,383,484; 6,376,654; 6,372,215; 6,359,126; 6,355,481; 6,355,444; 6,355,245; 6,355,244; 6,346,246; 6,344,198; 6,340,571; 6,340,459; 6,331,175; 6,306,393; 6,254,868; 6,187,287; 6,183,744; 6,129,914; 6,120,767; 6,096,289; 6,077,499; 5,922,302; 5,874,540; 5,814,440; 5,798,229; 5,789,554; 5,776,456; 5,736,119; 5,716,595; 5,677,136; 5,587,459; 5,443,953; 5,525,338, the Examples section of each of which is incorporated herein by reference. These are exemplary only and a wide variety of other antibodies and their hybridomas are known in the art. The skilled artisan will realize that antibody sequences or antibody-secreting hybridomas against almost any disease-associated antigen may be obtained by a simple search of the ATCC, NCBI and/or USPTO databases for antibodies against a selected disease-associated target of interest. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880, the Examples section of each of which is incorporated herein by reference).

In specific embodiments, the antibodies or antibody fragments used as the targeting ligands are specific for cancer antigens. Particular antibodies that may be of use for therapy of cancer within the scope of the present invention include, but are not limited to, LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD-1 receptor), nivolumab (anti-PD-1 receptor), ipilimumab (anti-CTLA-4), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM5), MN-15 or MN-3 (anti-CEACAM6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin) and trastuzumab (anti-ErbB2). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20050271671; 20060193865; 20060210475; 20070087001; the Examples section of each incorporated herein by reference.) Specific known antibodies of use include hPAM4 (U.S. Pat. No. 7,282,567), hA20 (U.S. Pat. No. 7,251,164), hA19 (U.S. Pat. No. 7,109,304), hIMMU-31 (U.S. Pat. No. 7,300,655), hLL1 (U.S. Pat. No. 7,312,318), hLL2 (U.S. Pat. No. 7,074,403), hMu-9 (U.S. Pat. No. 7,387,773), hL243 (U.S. Pat. No. 7,612,180), hMN-14 (U.S. Pat. No. 6,676,924), hMN-15 (U.S. Pat. No. 7,541,440), hR1 (U.S. patent application Ser. No. 12/772,645), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575) the text of each recited patent or application is incorporated herein by reference with respect to the Figures and Examples sections.

Other useful antigens that may be targeted include carbonic anhydrase IX, B7, CCCL19, CCCL21, CSAp, HER-2/neu, BrE3, CD1, CD11a, CD2, CD3, CD4, CD5, CD8, CD11A, CD14, CD15, CD16, CD18, CD19, CD20 (e.g., C2B8, hA20, 1F5 MAbs), CD21, CD22, CD23, CD25, CD29, CD30, CD32b, CD33, CD37, CD38, CD40, CD40L, CD44, CD45, CD46, CD47, CD52, CD54, CD55, CD59, CD64, CD67, CD70, CD74, CD79a, CD80, CD83, CD95, CD126, CD133, CD138, CD147, CD154, CEACAM5, CEACAM6, CTLA-4, alpha-fetoprotein (AFP), VEGF (e.g., AVASTIN®, fibronectin splice variant), ED-B fibronectin (e.g., L19), EGP-1 (TROP-2), EGP-2 (e.g., 17-1A), EGF receptor (ErbB1) (e.g., ERBITUX), ErbB2, ErbB3, Factor H, FHL-1, Flt-3, folate receptor, Ga 733, GRO-.beta., HMGB-1, hypoxia inducible factor (HIF), HM1.24, HER-2/neu, histone H2B, histone H3, histone H4, insulin-like growth factor (ILGF), IFN-γIFN-α, IFN-β, IFN-λ, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, the HLA-DR antigen to which L243 binds, CD66 antigens, i.e., CD66a-d or a combination thereof, MAGE, mCRP, MCP-1, MIP-1A, MIP-1B, macrophage migration-inhibitory factor (MIF), MUC1, MUC2, MUC3, MUC4, MUC5ac, placental growth factor (P1GF), PSA (prostate-specific antigen), PSMA, PAM4 antigen, PD-1 receptor, PD-L1, NCA-95, NCA-90, A3, A33, Ep-CAM, KS-i, Le(y), mesothelin, S100, tenascin, TAC, Tn antigen, Thomas-Friedenreich antigens, tumor necrosis antigens, tumor angiogenesis antigens, TNF-α, TRAIL receptor (R1 and R2), TROP-2, VEGFR, RANTES, T101, as well as cancer stem cell antigens, complement factors C3, C3a, C3b, C5a, C5, and an oncogene product.

For multiple myeloma therapy, suitable targeting antibodies have been described against, for example, CD38 and CD138 (Stevenson, 2006. Mol Med. 12(11-12):345-346; Tassone et al., 2004. Blood 104(12):3688-96), CD74 (Stein et al., 2007. Clin Cancer Res. 13(18 Pt 2):55565-55635.), CS1 (Tai et al., 2008. Blood 112(4):1329-37, and CD40 (Tai et al., 2005. Cancer Res. 65(13):5898-5906).

Macrophage migration inhibitory factor (MIF) is an important regulator of innate and adaptive immunity and apoptosis. It has been reported that CD74 is the endogenous receptor for MIF (Leng et al., 2003. J Exp Med 197:1467-76). The therapeutic effect of antagonistic anti-CD74 antibodies on MIF-mediated intracellular pathways may be of use for treatment of a broad range of disease states, such as cancers of the bladder, prostate, breast, lung, colon and chronic lymphocytic leukemia (e.g., Meyer-Siegler et al., 2004. BMC Cancer 12:34; Shachar and Haran, 2011. Leuk Lymphoma 52:1446-54); autoimmune diseases such as rheumatoid arthritis and systemic lupus erythematosus (Morand & Leech, 2005. Front Biosci 10:12-22; Shachar and Haran, 2011. Leuk Lymphoma 52:1446-54); kidney diseases such as renal allograft rejection (Lan, 2008. Nephron Exp Nephrol. 109:e79-83); and numerous inflammatory diseases (Meyer-Siegler et al., 2009. Mediators Inflamm epub Mar. 22, 2009; Takahashi et al., 2009. Respir Res 10:33; Milatuzumab (hLL1) is an exemplary anti-CD74 antibody of therapeutic use for treatment of MIF-mediated diseases.

Anti-TNF-α antibodies are known in the art and may be of use to treat immune diseases, such as autoimmune disease, immune dysfunction (e.g., graft-versus-host disease, organ transplant rejection) or diabetes. Known antibodies against TNF-α include the human antibody CDP571 (Ofei et al., 2011. Diabetes 45:881-85); murine antibodies MTNFα1, M2TNFAI, M3TNFAI, M3TNFABI, M302B and M303 (Thermo Scientific, Rockford, Ill.); infliximab (Centocor, Malvern, Pa.); certolizumab pegol (UCB, Brussels, Belgium); and Adalimumab (Abbott, Abbott Park, Ill.). These and many other known anti-TNF-α antibodies may be used as targeting ligands in the targeting constructs of the present invention. Other antibodies of use for therapy of immune dysregulatory or autoimmune disease include, but are not limited to, anti-B-cell antibodies such as veltuzumab, epratuzumab, milatuzumab or hL243; tocilizumab (anti-IL-6 receptor); basiliximab (anti-CD25); daclizumab (anti-CD25); efalizumab (anti-CD11a); muromonab-CD3 (anti-CD3 receptor); anti-CD4OL (UCB, Brussels, Belgium); natalizumab (anti-.alpha.4 integrin) and omalizumab (anti-IgE).

Checkpoint inhibitor antibodies have been used primarily in cancer therapy. Immune checkpoints refer to inhibitory pathways in the immune system that are responsible for maintaining self-tolerance and modulating the degree of immune system response to minimize peripheral tissue damage. However, tumor cells can also activate immune system checkpoints to decrease the effectiveness of immune response against tumor tissues. Exemplary checkpoint inhibitor antibodies against cytotoxic T-lymphocyte antigen 4 (CTLA4, also known as CD152), programmed cell death protein 1 (PD 1, also known as CD279) and programmed cell death 1 ligand 1 (PD-L1, also known as CD274), may be used in combination with one or more other agents to enhance the effectiveness of immune response against disease cells, tissues or pathogens. Exemplary anti-PD1 antibodies include lambrolizumab (MK-3475, MERCK), nivolumab (BMS-936558, BRISTOL-MYERS SQUIBB), AMP-224 (MERCK), and pidilizumab (CT-011, CURETECH LTD.). Anti-PD1 antibodies are commercially available, for example from ABCAM.RTM. (AB137132), BIOLEGEND® (EH 12.2H7, RMP1-14) and AFFYMETRIX EBIOSCIENCE (J105, J116, MIH4). Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Anti-PD-L1 antibodies are also commercially available, for example from AFFYMETRIX EBIOSCIENCE (MIH1). Exemplary anti-CTLA4 antibodies include ipilimumab (Bristol-Myers Squibb) and tremelimumab (PFIZER). Anti-PD1 antibodies are commercially available, for example from ABCAM® (AB134090), SINO BIOLOGICAL INC. (11159-H03H, 11159-H08H), and THERMO SCIENTIFIC PIERCE (PA5-29572, PA5-23967, PA5-26465, MA1-12205, MA1-35914). Ipilimumab has recently received FDA approval for treatment of metastatic melanoma (Wada et al., 2013, J Transl Med 11:89).

Type-1 and Type-2 diabetes may be treated using known antibodies against B-cell antigens, such as CD22 (epratuzumab and hRFB4), CD74 (milatuzumab), CD19 (hA19), CD20 (veltuzumab) or HLA-DR (hL243) (see, e.g., Winer et al., 2011. Nature Med 17:610-18). Anti-CD3 antibodies also have been proposed for therapy of type-1 diabetes (Cernea et a.l., 2010. Diabetes Metab Rev. 26:602-05).

When two or more targeting ligands are present in a targeting construct, such targeting ligands may be the same or different. In non-limiting embodiments in which the targeting ligands of an individual construct are different, the binding partners of the ligands represent different cognate binding partners of a target complex (e.g., a heteropolymeric complex, including a heteromultimeric macromolecule such as a heteromultimeric polypeptide). In illustrative example of this type, a target complex represents a receptor that comprises at least two different polypeptide chains. Such target complexes include heterodimeric and heterotrimeric receptor complexes, illustrative examples of which include type I cytokine receptors that comprise different polypeptide chains, some of which are involved in ligand/cytokine interaction are generally referred to the α-chains and others that are involved in signal transduction which include the β- andγ-chains. Non-limiting examples of α-chains include the α-chains of the interleukin-2 receptor, interleukin-3 receptor, interleukin-4 receptor, interleukin-5 receptor, interleukin-6 receptor, interleukin-7 receptor, interleukin-9 receptor, interleukin-11 receptor, interleukin-12 receptor, interleukin-13 receptor, interleukin-15 receptor, interleukin-21 receptor, interleukin-23 receptor, interleukin-27 receptor, colony stimulating factor receptors, erythropoietin receptor, GM-CSF receptor, G-CSF receptor, hormone receptor/neuropeptide receptor, growth hormone receptor, prolactin receptor, oncostatin M receptor and leukemia inhibitory factor). The signal transducing chains are often shared between different receptors within this receptor family. For example, the IL-2 receptor common γ-chain (also known as CD132) is shared between: IL-2 receptor, IL-4 receptor, IL-7 receptor, IL-9 receptor, IL-13 receptor and IL-15 receptor. The common β-chain (CD131 or CDw131) is shared between the following type I cytokine receptors: GM-CSF receptor, IL-3 receptor and IL-5 receptor. The gp230 receptor common y-chain (also known as gp130, IL6ST, IL6-beta or CD130) is shared between: IL-6 receptor, IL-11 receptor, IL-12 receptor, IL-27 receptor, leukemia inhibitory factor receptor and Oncostatin M receptor. In certain strategies, it is desirable to bind specifically with the α-chain of a cytokine receptor and to signal through a to least one different signal-transducing chain, in order to alleviate for example certain unwanted side effects associated with signaling through the signal-transducing chain(s) normally associated with the heteromultimeric complex, as for example described in U.S. Pat. App. Pub. No. 20140140949, which is hereby incorporated by reference herein in its entirety. In these embodiments, one of the targeting ligand is adapted to bind preferentially with the α-chain and at least one other targeting ligand is adapted to bind one or more signal-transducing chains not normally associated with the α-chain.

In specific embodiments, the targeting ligand is a scFv that binds with a target antigen selected from EGFR, mesothelin, Eph2a, VEGF, L1-CAM (CD171), OX-2 (CD200) and MUC1 (CD227), illustrative examples of which are shown in the targeting constructs illustrated in FIGS. 1 and 9.
4. Polymeric Vehicles/Assemblies

Depending on the polymer chain employed, the polymer chain is assembled with other polymer chains to form a polymeric vehicle, illustrative examples of which include particles such as but not limited to nanoparticles and microparticles. The particles, including nanoparticles and microparticles, are suitably selected from liposomes, micelles, filomicelles, lipoproteins, lipid-coated bubbles, polymersomes, niosomes, carbon nanoassemblies, paramagnetic particles, ferromagnetic particles, microvesicles, dendrimers and hyperbranched polymers. In preferred embodiments, the nanoparticles comprise hyperbranched polymers.

In certain embodiments, the polymeric vehicle is selected from microparticles or nanoparticles. In non-limiting examples of this type, the microparticles or nanoparticles are lipidic particles. Lipidic particles are microparticles or nanoparticles that include at least one lipid component forming a condensed lipid phase. Typically, a lipidic nanoparticle has preponderance of lipids in its composition. Various condensed lipid phases include solid amorphous or true crystalline phases; isomorphic liquid phases (droplets); and various hydrated mesomorphic oriented lipid phases such as liquid crystalline and pseudocrystalline bilayer phases (L-alpha, L-beta, P-beta, Lc), interdigitated bilayer phases, and nonlamellar phases (see, e.g., The Structure of Biological Membranes, ed. by P. Yeagle, CRC Press, Bora Raton, Fla., 1991). Lipidic microparticles include, but are not limited to a liposome, a lipid-nucleic acid complex, a lipid-drug complex, a lipid-label complex, a solid lipid particle, a microemulsion droplet, and the like. Methods of making and using these types of lipidic microparticles and nanoparticles are known in the art (see, e.g., U.S. Pat. Nos. 5,077,057; 5,100,591; 5,616,334; 6,406,713; 5,576,016; 6,248,363; Williams, A. P., Liposomes: A Practical Approach, 2n.sup.d Edition, Oxford Univ. Press (2003); Lasic, D. D., Liposomes in Gene Delivery, CRC Press LLC (1997); Bondi et al., 2003. Drug Delivery 10: 245-250; Pedersen et al., 2006. Eur J Pharm Biopharm. 62: 155-162, 2006 (solid lipid particles); U.S. Pat. Nos. 5,534,502; 6,720,001; Shiokawa et al., 2005. Clin Cancer Res. 11: 2018-2025 (microemulsions); U.S. Pat. No. 6,071,533 (lipid-nucleic acid complexes), and the like). In certain embodiments, methods can be used to produce liposomes that are multilamellar and/or unilamellar, which can include large unilamellar vesicles (LUV) and/or small unilamellar vesicles (SUV). Similar to self-assembly of liposomes in solution, micelles can be produced using techniques generally well known in the art, such that amphiphilic molecules will form micelles when dissolved in solution conditions sufficient to form micelles. Lipid-coated bubbles and lipoproteins can also be constructed using methods known in the art (See, e.g., Farook, 2009. R Soc Interface 6(32): 271-277); Lacko et al., Lipoprotein Nanoparticles as Delivery Vehicles for Anti-Cancer Agents in Nanotechnology for Cancer Therapy, CRC Press (2007)).

In specific embodiments, the polymeric vehicle is selected from polymeric microparticles or nanoparticles, which generally have several advantages including high stability, high carrier capacity, feasibility of incorporation of both hydrophilic and hydrophobic substances, and feasibility of variable routes of administration, including oral application and inhalation. Polymeric nanoparticles can also be designed to allow controlled (sustained) drug release from the matrix. Polymeric microparticles and nanoparticles are typically made from biocompatible and biodegradable materials. Methods of making polymeric nanoparticles that can be used in the present invention are generally well known in the art (see, e.g., Sigmund, W. et al., Eds., Particulate Systems in Nano- and Biotechnologies, CRC Press LLC (2009); Karnik et al., 2008. Nano Lett. 8(9):2906-2912). For example, block copolymers can be made using synthetic methods known in the art such that the block copolymers can self-assemble in a solution to form polymersomes and/or block copolymer micelles. Niosomes are known in the art and can be made using a variety of techniques and compositions (Baillie et al., 1988. J Pharm Pharmacol. 38:502-505). Magnetic and/or metallic particles can be constructed using any method known in the art, such as co-precipitation, thermal decomposition, and microemulsion. (See also Nagarajan, R. & Hatton, T. A., Eds., Nanoparticles Synthesis, Stabilization, Passivation, and Functionalization, Oxford Univ. Press (2008)).

Assembly of the polymeric vehicle may be directed, suitably by a cross-linking agent. Alternatively, the polymeric vehicle is assembled by self-assembly of the targeting constructs generally through their polymer chains.

The polymer chain may be naked. Alternatively, the polymer chain may be bound to or associated with a payload, typically when in the form a polymeric vehicle (which is also referred to herein as a “polymeric delivery vehicle”). In some embodiments, the payload comprises a therapeutic agent, illustrative examples of which include analgesics, anesthetics, anorexics, anti-allergics, antiarthritics, antiasthmatic agents, antibiotics, anticholinergics, anticonvulsants, antidepressants, antihemophilics, antidiabetic agents, antidiarrheals, antifungals, antigens, antihistamines, antihypertensives, anti-inflammatories, antimigraine preparations, antinauseants, antineoplastics, antiparkinsonism drugs, antiprotozoans, antipruritics, antipsychotics, antipyretics, antispasmodics, antivirals, calcium channel blockers, cardiovascular preparations, central nervous system stimulants, contraceptives, cough and cold preparations including decongestants, diuretics, enzyme inhibitors, enzymes, genetic material including DNA and RNA, growth factors, growth hormones, hormone inhibitors, hypnotics, immunonanobubbles, immunosuppressive agents, microbicides, muscle relaxants, parasympatholytics, peptides, peripheral and cerebral vasodilators, proteins, psychostimulants, receptor agonists, sedatives, spermicides and other contraceptives, steroids, sympathomimetics, tranquilizers, vaccines, vasodilating agents including general coronary, viral vectors, small organic molecules, and combinations thereof.

In specific embodiments, the therapeutic agents are selected from antibiotics, anti-restenotics, anti-proliferative agents, anti-neoplastic agents, chemotherapeutic agents, cardiovascular agents, anti-inflammatory agents, anti-hemophilic agents, immunosuppressive agents, anti-apoptotic and anti-tissue damage agents.

In the context of the present invention, an antibiotic is intended to include antibacterial, antimicrobial, antiviral, antiprotozoal and antifungal agents. Antiviral agents include, but are not limited to, nucleoside phosphonates and other nucleoside analogs, 5-amino-4-imidazolecarboxamide ribonucleotide (AICAR) analogs, glycolytic pathway inhibitors, anionic polymers, and the like, more specifically: antiherpes agents such as acyclovir, famciclovir, foscarnet, ganciclovir, idoxuridine, sorivudine, trifluridine, valacyclovir, and vidarabine; and other antiviral agents such as abacavir, adefovir, amantadine, amprenavir, cidofovir, delviridine, 2-deoxyglucose, dextran sulfate, didanosine, efavirenz, entecavir, indinavir, interferon alpha and PEGylated interferon, interferon alfacon-1, lamivudine, nelfinavir, nevirapine, ribavirin, rimantadine, ritonavir, saquinavir, squalamine, stavudine, telbivudine, tenofovir, tipranavir, valganciclovir, zalcitabine, zidovudine, zintevir, and mixtures thereof. Still other antiviral agents are glycerides, particularly monoglycerides, which have antiviral activity. One such agent is monolaurin, the monoglyceride of lauric acid.

Antimicrobial agents include, e.g., those of the lincomycin family, such as lincomycin per se, clindamycin, and the 7-deoxy,7-chloro derivative of lincomycin (i.e., 7-chloro-6,7,8-trideoxy-6-[[(1-methyl-4-propyl-2-pyrrolidinyl)carbonyl]amino]-1-thio-L-threo-alpha-D-galacto-octopyranoside); other macrolide, aminoglycoside, and glycopeptide antimicrobials such as erythromycin, clarithromycin, azithromycin, streptomycin, gentamicin, tobramycin, amikacin, neomycin, vancomycin, and teicoplanin; antimicrobials of the tetracycline family, including tetracycline per se, chlortetracycline, oxytetracycline, demeclocycline, rolitetracycline, methacycline and doxycycline; and sulfur-based antimicrobials, such as the sulfonamides sulfacetamide, sulfabenzamide, sulfadiazine, sulfadoxine, sulfamerazine, sulfamethazine, sulfamethizole, and sulfamethoxazole; streptogramin antimicrobials such as quinupristin and dalfopristin; and quinolone antibiotics such as ciprofloxacin, nalidixic acid, ofloxacin, and mixtures thereof.

Antifungal agents include, e.g., miconazole, terconazole, isoconazole, itraconazole, fenticonazole, fluconazole, ketoconazole, clotrimazole, butoconazole, econazole, metronidazole, 5-fluorouracil, amphotericin B, and mixtures thereof.

Other anti-infective agents include miscellaneous antibacterial agents such as chloramphenicol, spectinomycin, polymyxin B (colistin), and bacitracin, anti-mycobacterials such as such as isoniazid, rifampin, rifabutin, ethambutol, pyrazinamide, ethionamide, aminosalicylic acid, and cycloserine, and antihelminthic agents such as albendazole, oxfendazole, thiabendazole, and mixtures thereof. Representative examples of antiprotozoal agents include pentamidine isethionate, quinine, chloroquine, and mefloquine.

Representative examples of restenosis therapeutic agents include, for example, anti-angiogenic agents such as anti-invasive factor (Eisentein et al., 1975. Am J Pathol. 81:337-346; Langer et al., 1976. Science 193:70-72; Horton et al., 1978. Science 199:1342-1345), retinoic acid and derivatives thereof which alter the metabolism of extracellular matrix components to inhibit angiogenesis, tissue inhibitor of metalloproteinase-1, tissue inhibitor of metalloproteinase-2, plasminogen activator inhibitor-1, plasminogen activator inhibitor-2, and anginex (Griffioen et al., 2001. Biochem J. 354(Pt 2):233-42); collagen inhibitors such as halofuginone or batimistat; antisense oligonucleotides directed to nucleic acid sequences encoding c-myc or c-myb; growth factor inhibitors such as tranilast, trapidil or angiopeptin; antioxidants such as probucol, anti-thromobotics such as heparin or abciximab, anti-proliferative agents such as AG-1295 (Fishbein et al., 2000. Arterioscler Thromb Vasc Biol. 20:667), tyrphostin (Banai et al., 2005. Biomaterials 26(4):451-61), pacitaxel or other taxanes (Scheller et al., 2004. Circulation 110(7):810-4), isoflavones (Kanellakis et al., 2004. Atherosclerosis 176(1):63-72), rapamycin or derivatives or analogs thereof (Schachner et al., 2004. Ann Thorac Surg. 77(5):1580-5), vincristine, vinblastine, HMG-CoA reductase inhibitors, doxorubicin, colchicines, actinomycin D, mitomycin C, cyclosporine, or mycophenolic acid; anti-inflammatory agents such as dexamethasone (Liu et al., 2004. Expert Rev Cardiovasc Ther. 2(5):653-60), methylprednisolone, or gamma interferon; and the like which exhibits anti-restenotic activity.

Other therapeutic agents that can be utilized in accordance with the present invention include anti-proliferative, anti-neoplastic or chemotherapeutic agents to prevent or treat tumors. Representative examples of such agents include androgen inhibitors; antiestrogens and hormones (e.g., flutamide, leuprolide, tamoxifen, estradiol, estramustine, megestrol, diethylstilbestrol, testolactone, goserelin, medroxyprogesterone); cytotoxic agents (e.g., altretamine, bleomycin, busulfan, carboplatin, carmustine (BiCNU), cisplantin, cladribine, dacarbazine, dactinomycin, daunorubicin, doxorubicin, estramustine, etoposide, lomustine, cyclophosphamide, cytarabine, hydroxyurea, idarubicin, interferon alpha-2a and -2b, ifosfamide, mitoxantrone, mitomycin, paclitaxel, streptozocin, teniposide, thiotepa, vinblastine, vincristine, vinorelbine); antimetabolites and antimitotic agents (e.g., floxuridine, 5-fluorouracil, fluarabine, interferon alpha-2a and -2b, leucovorin, mercaptopurine, methotrexate, mitotane, plicamycin, thioguanine, colchicines); folate antagonists and other anti-metabolites; vinca alkaloids; nitrosoureas; DNA alkylating agents; purine antagonists and analogs; pyrimidine antagonists and analogs; alkyl solfonates; enzymes (e.g., asparaginase, pegaspargase); and toxins (e.g., ricin, abrin, diphtheria toxin, cholera toxin, gelonin, pokeweed antiviral protein, tritin, Shigella toxin, and Pseudomonas exotoxin).

Further therapeutic agents that can be utilized within the present invention include cardiovascular agents such as antihypertensive agents; adrenergic blockers and stimulators (e.g., doxazosin, guanadrel, guanethidine, pheoxybenzamine, terazosin, clonidine, guanabenz); alpha-/beta-adrenergic blockers (e.g., labetalol); angiotensin converting enzyme (ACE) inhibitors (e.g., benazepril, catopril, lisinopril, ramipril); ACE-receptor antagonists (e.g., losartan); beta blockers (e.g., acebutolol, atenolol, carteolol, pindolol, propranolol, penbatolol, nadolol); calcium channel blockers (e.g., amiloride, bepridil, nifedipine, verapamil, nimodipine); antiarrythmics, groups I-IV (e.g., bretylium, lidocaine, mexiletine, quinidine, propranolol, verapamil, diltiazem, trichlormethiazide, metoprolol tartrate, carteolol hydrochloride); and miscellaneous antiarrythmics and cardiotonics (e.g., adenosine, digoxin, caffeine, dopamine hydrochloride, digitalis).

Other therapeutic agents that can be used in accord with the present invention include anti-inflammatory agents. Representative examples of such agents include nonsteroidal agents (NSAIDS) such as salicylates, diclofenac, diflunisal, flurbiprofen, ibuprofen, indomethacin, mefenamic acid, nabumetone, naproxen, piroxicam, ketoprofen, ketorolac, sulindac, tolmetin. Other anti-inflammatory drugs include steroidal agents such as beclomethasone, betamethasone, cortisone, dexamethasone, fluocinolone, flunisolide, hydrorcortisone, prednisolone, and prednisone. Immunosuppressive agents are also contemplated (e.g., adenocorticosteroids, cyclosporin). Exemplary corticosteroids include e.g., lower potency corticosteroids such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, or methylprednisolone, or higher potency corticosteroids such as clobetasol propionate, betamethasone benzoate, betamethasone diproprionate, diflorasone diacetate, fluocinonide, mometasone furoate, triamcinolone acetonide, and mixtures thereof.

Antihemophilic agents include, e.g., antifibrinolytic amino acids, aprotinin, 1-deamino-8-d-arginine vasopressin, aminocaproic acid, tranexamic acid and conjugated estrogens, and mixtures thereof (Mannucci et al., 1998. New Eng J Med. 339:245).

Further therapeutic agents include anti-tissue damage agents. Representative examples of such agents include superoxide dismutase; immune modulators (e.g., lymphokines, monokines, interferon α and β); and growth regulators (e.g., IL-2, tumor necrosis factor, epithelial growth factor, somatrem, fibronectin, GM-CSF, CSF, platelet-derived growth factor, somatotropin, rG-CSF, epidermal growth factor, IGF-1).

In a particular embodiment, the therapeutic agent is an anti-restenotic agent such as rapamycin (i.e., sirolimus) or a derivative or analog thereof, e.g., everolimus or tacrolimus (Grube et al., 2004. Circulation 109(18):2168-71; Grube and Buellesfeld, 2004. Herz 29(2):162-6).

In another embodiment, the therapeutic agent is an anti-apoptotic agent such as Galectin-3; (-)deprenyl; monoamine oxidase inhibitors (MAO-I) such as selegiline and rasagiline; Rapamycin; or quercetin.

Alternatively, or in addition, the payload comprises an imaging agent, non-limiting examples of which include a fluorescent label, a near infrared label, a luminescent label, a bioluminescent label, a magnetic label, a chemiluminescent label, a radioisotope, and a contrast agent for magnetic resonance imaging.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly useful. Ions useful in other contexts, such as X-ray imaging, include but are not limited to lanthanum (III), gold (III), lead (II), and especially bismuth (III).

Radioisotopes of potential use as imaging (and in some embodiments therapeutic agents) include astatine²¹¹, ¹⁴carbon, ⁵¹chromium, ³⁶chlorine, ⁵⁷cobalt, ⁵⁸cobalt, copper⁶⁴, copper67, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium⁹⁹m, yttrium⁹⁰, zirconium⁸⁹ and ¹²⁵I is often being employed for use in certain embodiments, and technicium⁹⁹m and indium¹¹¹ are also often utilized due to their low energy and suitability for long range detection.

In certain embodiments, imaging agents have excitation and emission wavelengths in the red and near infrared spectrum in the range 550-1300 or 400-1300 nm or about 440 and about 1100 nm, between about 550 and about 800 nm, between about 600 and about 900 nm. Use of this portion of the electromagnetic spectrum maximizes tissue penetration and minimizes absorption by physiologically abundant absorbers such as hemoglobin (<650 nm) and water (>1200 nm). Such optical imaging probes with excitation and emission wavelengths in other spectrums, such as the visible and ultraviolet light spectrum, can also be employed in the methods of the present invention. In particular, fluorophores such as certain carbocyanine or polymethine fluorescent fluorochromes or dyes can be used to construct optical imaging agents, e.g., U.S. Pat. No. 6,747,159; U.S. Pat. No. 6,448,008; U.S. Pat. No. 6,136,612; U.S. Pat. No. 4,981,977; U.S. Pat. No. 5,268,486; U.S. Pat. No. 5,569,587; U.S. Pat. No. 5,569,766; U.S. Pat. No. 5,486,616: U.S. Pat. No. 5,627,027; U.S. Pat. No. 5,808,044; U.S. Pat. No. 5,877,310; U.S. Pat. No. 6,002,003; U.S. Pat. No. 6,004,536; U.S. Pat. No. 6,008,373; U.S. Pat. No. 6,043,025; U.S. Pat. No. 6,127,134; U.S. Pat. No. 6,130,094; U.S. Pat. No. 6,133,445; also WO 97/40104, WO 99/51702, WO 01/21624, and EP 1 065 250 A1; and Tetrahedron Letters 41, 9185-88 (2000), which are incorporated herein by reference.

In some embodiments, the imaging agents have excitation and emission wavelengths in near infrared (NIR) spectrum, illustrative examples of which include BODIPY® fluorophores (Molecular Probes) (e.g., 4,4-difluoro-4-bora-3a,4a-diaza-s-indacene (and derivatives thereof), which can be modified to alter the wavelength (BODIPY® substitutes for the fluorescein, rhodamine 6G, tetramethylrhodamine and Texas Red fluorophores are BODIPY® FL, BODIPY®TM R6G, BODIPY® TMR and BODIPY® TR, respectively)), 1H, 5H, 11H, 15H-Xantheno[2,3,4-ij:5,6,7-i′j′]diquinolizin-18-ium, 9-[2(or 4)-(chlorosulfonyl)-4(or 2)-sulfophenyl]-2,3,6,7,12,13,16,17-octahydro-, inner salt (molecular formula: C31H29CIN2O6S2) (and derivatives thereof) (Texas Red), Xanthylium, 3,6-diamino-9-(2-(methoxycarbonyl) phenyl, chloride (C₂₁H₁₇CIN₂O₃) (and derivatives thereof) (NIR Rhodamine dye), and cyanine dyes (and derivatives thereof), where derivatives of each can be used to modify the wavelength. In particular, the fluorescent compound can include, but is not limited to, BODIPY® dye series (e.g., BODIPY® FL-X BODIPY® R6G-X, BODIPY® TMR-X, BODIPY® TR-X, BODIPY® 630/650-X, and BODIPY® 650/665-X (Molecular Probes, Inc. Eugene, Oreg., USA)), NIR Rhodamine dyes, NIR ALEXA® dyes (e.g., ALEXA® Fluor 350, ALEXA® Fluor 405, ALEXA® Fluor 430, ALEXA® Fluor 488, ALEXA® Fluor 500 (Molecular Probes, Inc. Eugene, Oreg., USA)), Texas Red, or cyanine dyes (e.g., Cy5.5 Cy3, Cy5), and Li-Cor IRDye™ products.

Another group of suitable imaging probes are lanthanide metal-ligand probes. Fluorescent lanthanide metals include europium and terbium. Fluorescence properties of lanthanides are described in Lackowicz, 1999, Principles of Fluorescence Spectroscopy, 2^{nd} Ed., Kluwar Academic, New York, the relevant text incorporated by reference herein.
5. Pharmaceutical Compositions and Formulations

In exemplary embodiments, the targeting constructs and polymeric vehicles of the present invention can be formulated into pharmaceutical compositions, along with a pharmaceutically acceptable carrier, diluent, or excipient. In this regard, the invention further provides pharmaceutical compositions comprising a targeting construct or polymeric vehicle of the invention, which composition is intended for administration to a subject, e.g., a mammal.

In some embodiments, the targeting construct or polymeric vehicle is present in the pharmaceutical composition at a purity level suitable for administration to a subject. In some embodiments, the targeting construct or polymeric vehicle has a purity level of at least about 90%, about 91%, about 92%, about 93%, about 94%, about 95%, about 96%, about 97%, about 98% or about 99%, and a pharmaceutically acceptable diluent, carrier or excipient.

Depending on the route of administration, the particular targeting construct or polymeric vehicle intended for use, as well as other factors, the pharmaceutical composition may comprise additional pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface nanobubbles, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety, which discloses various components used in formulating pharmaceutically acceptable compositions and known techniques for the preparation thereof.
5.1 Routes of Administration

The targeting constructs or polymeric vehicles, or pharmaceutical composition comprising them, may be administered to the subject via any suitable route of administration. The following discussion on routes of administration is merely provided to illustrate exemplary embodiments and should not be construed as limiting the scope in any way.

Formulations suitable for oral administration can consist of (a) liquid solutions, such as an effective amount of the targeting construct or polymeric vehicle of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the targeting construct or polymeric vehicle of the present invention in a flavor, usually sucrose and acacia or tragacanth, as well as pastilles comprising the targeting constructs or polymeric vehicles of the present invention in an inert base, such as gelatin and glycerin, or sucrose and acacia, emulsions, gels, and the like containing, in addition to, such excipients as are known in the art.

The targeting construct or polymeric vehicle of the present invention can be delivered, whether alone or in combination with other suitable components, via pulmonary administration and can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa. In some embodiments, the targeting construct or polymeric vehicle is formulated into a powder blend or into microparticles or nanoparticles. Suitable pulmonary formulations are known in the art (see, e.g., Qian et al., 1009. Int J Pharm. 366:218-220; Adjei and Garren, 1990. Pharmaceutical Research 7(6):565-569 (1990); Kawashima et al., 1999. J Controlled Release 62(1-2):279-287; Liu et al., 1993. Pharm Res. 10(2):228-232; International Patent Application Publication Nos. WO 2007/133747 and WO 2007/141411.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous. The targeting construct or polymeric vehicle of the present disclosure can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-I53-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments contain from about 0.5% to about 25% by weight of the targeting construct or polymeric vehicle of the present invention in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

Injectable formulations are also contemplated. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Additionally, the targeting construct or polymeric vehicle of the invention can be made into suppositories for rectal administration by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.
5.2 Dosages

The targeting constructs and polymeric vehicles of the present invention are believed to be useful in methods of treating various diseases and conditions in a subject, and other methods, as described herein. Non-limiting conditions include pathogenic infections, stenosis, hyperproliferative disease such as cancer, inflammatory disorders, cardiovascular disorders including hypertension and stenosis, wounds, hematological disorders, coagulation disorders such as hemophilia and autoimmune diseases.

For purposes of the present invention, the amount or dose of the targeting construct or polymeric vehicle administered should be sufficient to effect, e.g., a therapeutic or prophylactic response, in the subject or animal over a reasonable time frame. For example, the dose of the targeting construct or polymeric vehicle should be sufficient to treat cancer as described herein in a period of from about 1 to 4 min, 1 to 4 hr or 1 to 4 wk or longer, e.g., 5 to 20 or more wk, from the time of administration. In certain embodiments, the time period could be even longer. The dose will be determined by the efficacy of the particular targeting construct or polymeric vehicle and the condition of the animal (e.g., human), as well as the body weight of the animal (e.g., human) to be treated.
5.3 Controlled Release Formulations

In some embodiments, the targeting construct or polymeric vehicle described herein can be modified into a depot form, such that the manner in which the targeting construct or polymeric vehicle of the invention is released into the body to which it is administered is controlled with respect to time and location within the body (see, e.g., U.S. Pat. No. 4,450,150). Depot forms of targeting construct or polymeric vehicle can be, for example, an implantable composition comprising the targeting construct or polymeric vehicle and a porous or non-porous material, such as a polymer, wherein the targeting construct or polymeric vehicle is encapsulated by or diffused throughout the material and/or degradation of the non-porous material. The depot is then implanted into the desired location within the body of the subject and the targeting construct or polymeric vehicle is released from the implant at a predetermined rate.

The pharmaceutical composition comprising the targeting construct or polymeric vehicle in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art, and may be applicable to such controlled release formulations comprising targeting construct or polymeric vehicle (see, e.g., Qian et al., 2009. J Pharm. 374:46-52; and International Patent Application Publication Nos. WO 2008/130158, WO2004/033036; WO2000/032218; and WO 1999/040942).
5.4Timing of Administration

The pharmaceutical compositions and formulations may be administered according to any regimen including, for example, daily (1 time per day, 2 times per day, 3 times per day, 4 times per day, 5 times per day, 6 times per day), every two days, every three days, every four days, every five days, every six days, weekly, bi-weekly, every three weeks, monthly, or bi-monthly. Timing, like dosing can be fine-tuned based on dose-response studies, efficacy, and toxicity data, and initially gauged based on timing used for other nanoparticle/microparticle-based therapeutics.
6. Diagnostic Methods

Provided herein are diagnostic-type methods utilizing the targeting constructs and polymeric vehicles of the invention. For example, a method of determining the presence of a disease or condition in a subject is provided. The method comprises administering to the subject a targeting construct or polymeric vehicle of the invention, wherein the targeting construct or polymeric vehicle comprises an imaging agent. In exemplary aspects, the targeting construct or polymeric vehicle administered to the subject and the method further comprises imaging the targeting construct or polymeric vehicle in the subject. In exemplary aspects, the targeting construct or polymeric vehicle comprises a cancer cell-targeting ligand, such as any of those described herein.

In other embodiments, the targeting construct with specificity to an analyte (e.g., a substance associated with the disease or condition) is contacted with a biological sample
7. Kits

The targeting constructs and polymeric vehicles of the invention may be provided as a kit or a package or unit dose. As used herein, the term “unit dose” is a discrete amount of a composition, e.g., a therapeutic composition or a diagnostic composition dispersed in a suitable carrier. Accordingly, the invention further provides kits, packages, and unit doses, each of which comprises a targeting construct or polymeric vehicle as broadly described herein.

In exemplary embodiments, the components of the kit/unit dose are packaged with instructions for administration to a subject, e.g., a human. Suitably, the kit comprises one or more devices for administration to a subject, e.g., a needle and syringe, a dropper, a measuring spoon or cup or like device, an inhaler, and the like. In some embodiments, the targeting construct or polymeric vehicle are pre-packaged in a ready to use form, e.g., a syringe, an intravenous bag, an inhaler, a tablet, capsule, etc. In some aspects, the kit further comprises other therapeutic or diagnostic agents or pharmaceutically acceptable carriers (e.g., solvents, buffers, diluents, etc.), including any of those described herein.

The following examples are given merely to illustrate the present invention and not in any way to limit its scope.

### EXAMPLES

### Example 1

### Bispecific Anti-PEG—Anti-Recpetor Antibodies

Production and Purification of anti PEG BsAbs

Bispecific antibody (BsAb) fragments were developed, incorporating a single chain variable region specific for PEG (PEG scFv) and a variable region specific for other receptors such as EGFR, VEGFR2, EphA2 and mesothelin. Both scFvs of an individual bispecific antibody are linked by a glycine serine (G4S) linker. Genes encoding bispecific antibody fragments which bind to PEG and various target receptors (EGFR, EphA2, VEGFR2 and mesothelin) were synthesized by Geneart. BsAb genes were codon optimized for expression in Chinese hamster ovary (Cricetulus griseus) cells, and designed with an immunoglobulin κ light chain leader sequence enabling the secretion of the BsAb from the cells into the cell medium. A 6xHistidine motif at the N-terminus of the BsAb and a c-myc epitope tag at the C-terminus were included to facilitate purification and detection of the BsAb following expression. The BsAb sequences are shown in FIG. 1. The BsAb genes were cloned into pcDNA 3.1 (+) mammalian expression plasmid (Invitrogen) using HindIII and NotI restriction sites. Plasmid DNA was transfected into CHO-S cells using 2 μg of DNA per mL of cells at a concentration of 3 million cells/mL. For transfection plasmid DNA was complexed with polyethylenimine-Pro (PolyPlus) in Opti-Pro serum free medium (Life Technologies) at a DNA (μg) to PEI (μL) ratio of 1:4 for 15 min prior to transfecting suspension adapted Chinese hamster ovary (CHO) cells. 2 μg of DNA was transfected per mL of CHO cells, which were at a cell density of 3 million cells/mL. The transfected cells were cultured in chemically defined CHO medium (CD-CHO; Life Technologies) at 37° C., 7.5% CO₂, 70% humidity with shaking at 130 rpm for 6 hr, before feeding with CD CHO Efficient Feeds A (Life Technologies), CD-CHO Efficient Feed B (Life Technologies) and anti-clumping agent (Gibco) and continuing the culture at 32° C., 7.5% CO₂, 70% humidity with shaking at 130 rpm for 7-14 days. Viability of the cells was evaluated by trypan blue staining from Day 7 onwards, and culturing was stopped when viability decreased below 50%.

Following transfection, the cells were pelleted by centrifugation at 5250 g for 30 min and the supernatant was collected and filtered through a 0.22 μm membrane (Sartorius). The BsAbs were purified from the supernatant utilizing a 5 mL Histrap excel column (GE Healthcare), eluting the protein with 20 mM sodium phosphate, 500 mM sodium chloride and 500 mM Imidazole pH 7.4, followed by buffer exchange into phosphate buffered saline pH 7.4 using the HiPrep 26/10 column (GE Healthcare). The final product was filtered through a 0.22 μm membrane and the concentration was determined by measuring protein absorbance at 280 nm using the Nanodrop 1000 and protein was further analyzed by SDS PAGE using 4-12% Bis-Tris gels (Invitrogen) and size exclusion HPLC using the TSK gel G3000SW column (Tosoh) and stored at −20° C.

### Target Binding ELISA

The target binding of the BsAbs was evaluated by indirect ELISA methods using PEG polymer and recombinant target proteins immobilized on ELISA plates. Individual wells of a 96 well Maxisorp plate were coated with 100 μL of 10 μg/mL of polymer (polyethylene glycol monomethyl ether methacrylate) or 10 μg/mL of target recombinant receptor (EphA2, VEGFR2, Mesothelin and EGFR) for 16-20 hr at 4C. PEG and target receptors were diluted in phosphate buffered saline (PBS pH 7.4). Following coating, the solution was decanted and 200 μL of 2% skim milk in PBST (PBS±0.05% Tween 20) was added to each well for 60 min to block non-specific binding. The blocker was decanted and 100 μL of BsAb, either in cell culture supernatant or purified protein stored in PBS, was added to each well. The BsAb was incubated for 2 hr and then decanted. The wells were washed five times manually in PBST and 100 μL of HRP labeled anti-c-myc antibody diluted 1/5000 in blocker was added to each well and incubated for 30 mins. The c-myc antibody was then decanted and the wells washed again five times manually with PBST. One hundred microliters of TMB was added to all wells and incubated for 10-15 mins or until adequate color development was identified. The TMB colorimetric reaction was neutralized by adding 100 μL of 2M sulfuric acid. The colorimetric reactions in each well was analyzed at an absorbance of 450 nm using the Spectramax plate reader.

### Competitive Binding ELISA

A competitive binding ELISA was used to determine the concentration of free polymer required to inhibit the binding of BsAb to immobilized polymer. Purified BsAb was diluted in PBS to 10 μg/mL and mixed with 48 μg/mL, 4.8 μg/mL and 0.48 μg/mL polymer for 60 mins. BsAb-polymer mixes were added to ELISA plates coated with 10 μg/ml polymer and target binding ELISA protocol was followed.

### Flow Cytometry

100 μL of BsAb at a concentration of 200 μg/mL in 10% FCS-PBS was mixed with 100 μL each of PEG polymers at concentrations of 2700 μg/mL, 540 μg/mL, 270 μg/mL and 135 μg/mL. BsAb was also mixed with PBS alone. PEG polymer concentrations were prepared in PBS. BsAb and polymers were incubated for 60mins at room temperature, then reactions were added to 100 μL of MDA-MB-468 cells, which overexpress EGFR and incubated for 1 hr at 4° C. MDA-MB-468 cells were prepared at 1-2 million cells/mL in 10% FCS-PBS. Following incubation, the cells were centrifuged gently at 1000 rpm for 5 mins, the supernatant was pipetted off and 100 μL of 10% FCS-PBS added. This wash step was repeated two more times. After the third and final wash, the supernatant was removed from the cells and the pellet was resuspended in 100 μL 10% FCS-PBS. The cells were analyzed by flow cytometry at 660 nm using 660+20 Red A filter.

In vivo Imaging

Hyperbranched polymers constructed from polyethyleneglycol monomethylether methacrylate (PEGMA) were labeled with a fluorophore for molecular imaging (see, for example, Pearce et al., 2014. Polymer Chemistry; Boase et al., 2014. Polymer Chemistry 5(15):4387). A model cell line that overexpresses the EphA2 receptor was used for investigating the in vivo imaging potential of the construct.

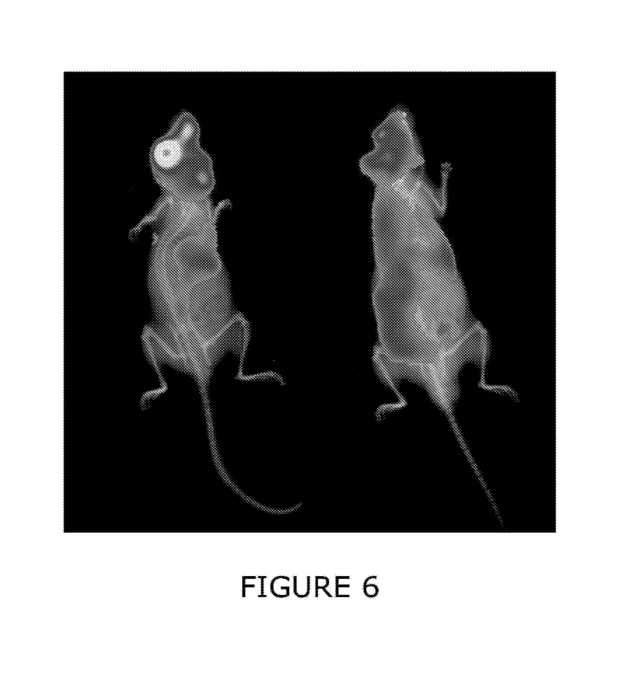

An orthotopic glioma model (as described, for example, Day et al., 2013. Cancer Cell 23:238-248) was utilized in which U87 cancer cells were injected into the brain of NOD/Skid mice. The formation of a solid tumor was verified over 3-4 wk after which the imaging was performed. Prior to injection of the diagnostic, 100 μg of fluorophore-labeled polymer was incubated for 30 min with 300 μg anti-PEG-anti-EphA2 bispecific antibody. Final solution had a concentration of 2 mg/mL. 100 pL of this solution was injected into the tail vein of the mouse and the mouse was imaged at various time points following injection. As a control, the polymer was injected into a different mouse without first being incubated with the anti-PEG-anti-EphA2 bispecific. FIG. 6 shows the distribution of polymer within the mouse 24 hr post-injection by optical imaging, where the targeted polymer containing the bispecific antibody clearly is accumulating in the tumor, while the untargeted material has cleared from the animal.

### RESULTS

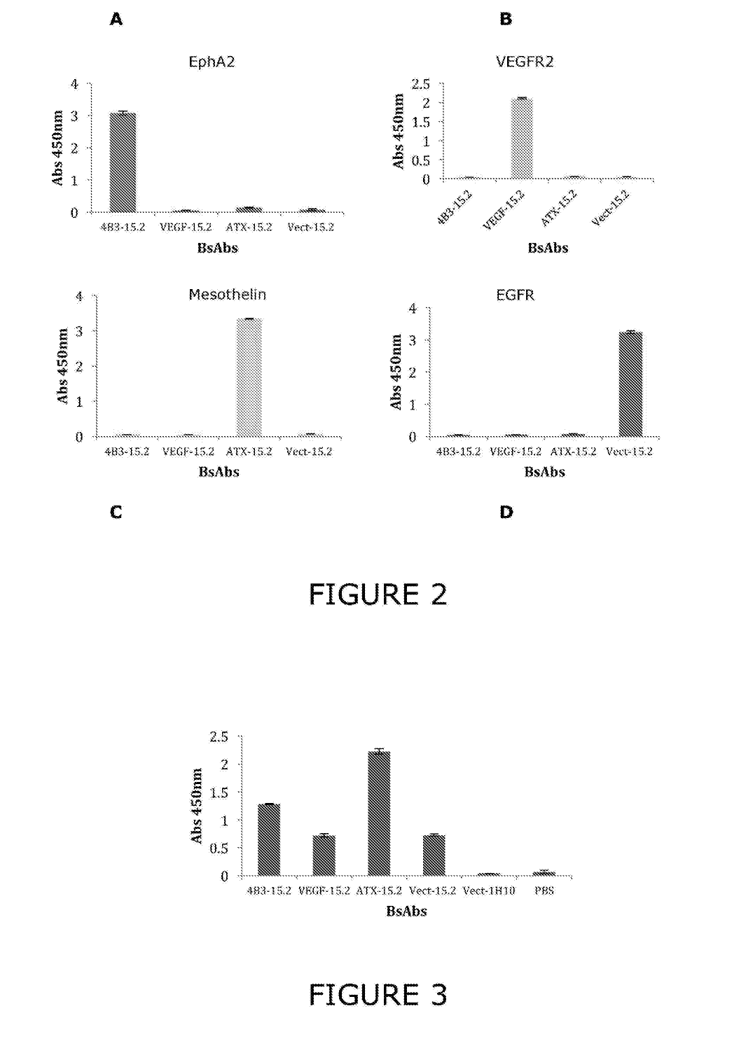

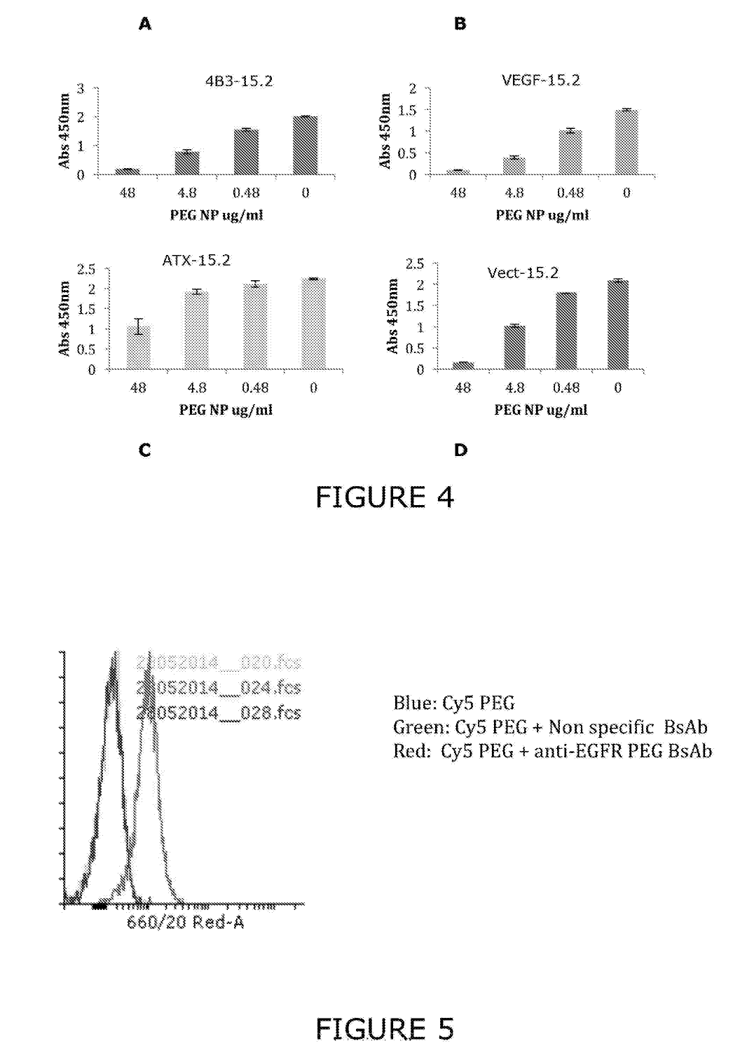

ELISA results indicated that each BsAb interacted with its specific recombinant target with no cross-reactivity with the other targets (FIG. 2). It was also evident by ELISA that all PEG BsAbs interacted with PEG polymer (FIG. 3). Competitive binding ELISAs indicated that premixing 48 μg/mL of free polymer with 10 μg/mL BsAb resulted in complete blocking of BsAb binding to immobilized polymer, with the exception of the PEG BsAb targeting mesothelin, which has 50% blocking (FIG. 4). FACs analysis with the PEG-EGFR BsAb conjugated to Cy5 labeled PEG polymer indicated that the BsAb could target the polymer specifically to native EGFR expressed on MDA-MB-468 cells (FIG. 5).

In this example, the term “PEG” in “PEG”, “PEG polymer”, “anti-PEG BsAbs”, “PEG BsAbs”, “bispecific anti-PEG”, “PEG scFv”, “PEG-EGFR BsAb” and “anti-PEG-anti-EphA2” refers to methoxy PEG.

### Example 2

### Binding of EGFR-PEG BsAb to Linear and Hyperbranched mPEG using BLI

The binding affinity of the EGFR-PEG BsAb for HBP and rEGFR was determined using biolayer interferometry (BLI), a label free, biosensor-based method to measure real time interactions between an immobilized ligand (HBP or rEGFR) and analyte (BsAb) in solution (Concepcion et al. 2009. Combinatorial Chemistry & High Throughput Screening 12 (8): 791-800). BLI determined that the BsAb had strong binding affinity in the nM range for both targets.

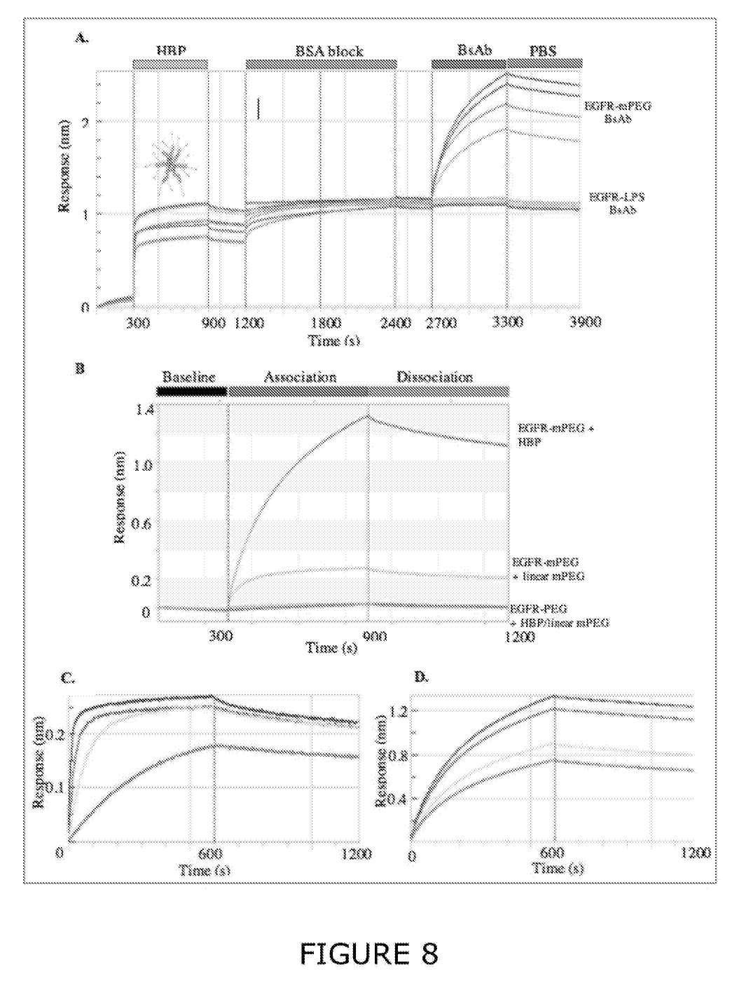

To determine binding affinity of BsAb for HBP using BLI, biosensors were coated with HBP, sensors were blocked with bovine serum albumin (BSA) and then BsAb binding measured (FIG. 8A). The EGFR-mPEG BsAb displayed a K_{D} of 1×10⁻⁸ M for HBP and Cy5 labeled HBP. This is comparable with other studies using PEG BsAbs constructed from complete IgG1 immunoglobulins, which have a K_{D} of 1.9 to 3.0×10⁻⁸ M for linear PEG chains as determined by microscale thermophoresis ²⁸. The binding of EGFR-PEG BsAb to linear mPEG (Mw 2000) was also demonstrated (FIG. 8B).

Similar binding affinities were demonstrated for the HBP and linear PEG using BLI, which is not surprising as the same methoxy PEG epitope is being targeted in all cases. While linear and HBPs show similar affinities to the BsAb, it is anticipated that the BsAbs will have higher avidity for the hyperbranched mPEG owing to the much higher density of methoxy PEG epitopes. Initial BLI indicates a 6-fold increase in the BsAb binding response to HBP compared to linear mPEG (FIG. 8B). Both PEG structures were saturated on BLI biosensors however in order to decouple the two different factors of affinity and avidity. The BLI sensorgram for EGFR-PEG binding to HBP demonstrated a slow association in comparison to its rapid binding to EGFR (FIG. 8C). Importantly, there was no binding to HBP by the EGFR-LPS BsAb as well as another EGFR-PEG BsAb specific for hydroxy PEG backbone (FIG. 8B). Likewise, the EGFR-mPEG BsAb failed to bind LPS in ELISA (FIG. 8B), indicating specific binding to HBP.

To determine the affinity of BsAb for EGFR, anti-Fc sensors were used to capture EGFR-Fc and then BsAb binding response determined. The BsAb displayed a binding constant (K_{D}) of 1×10⁻⁹M for immobilized rEGFR (FIG. 8C).

In this example, the term “PEG” in “EGFR-PEG BsAb” refers to methoxy PEG.

### Example 3

### Development of BsAbs Targeting Novel Cancer Cell Surface Antigens

Three further BsAbs were designed to target novel potential cancer cell surface markers, CD171 (L1 cell adhesion molecule; L1CAM), CD200 (OX-2 membrane glycoprotein; OX-2), and CD227 (mucin 1; MUC1).

Initial development of these BsAbs involved the identification of the variable heavy (see, amino acid sequences in bold typeface, white background, FIG. 9) and light domains (see, amino acid sequences regular typeface, white background, FIG. 9) from sequences of monoclonal antibodies with affinity to MUC1, OX-2 and L1CAM. The variable heavy (see, amino acid sequences in bold typeface, gray background, FIG. 9) and light chain (see, amino acid sequences regular typeface, gray background, FIG. 9) domains of the anti-mPEG scFv sequences are the same ones used in Example 1. The derived scFvs were linked by a G4S linker. His and c-myc tags (cyan) were also added to bispecific antibody to allow for easy purification and detection in assays.

### Example 4

### Anti-CD171-PEG (L1_9.3Hu—15-2) BsAb

The anti-CD171 scFv was designed from variable regions identified from the humanized monoclonal antibody published by Kelm et al. (2012, U.S. Pat. No. 8,138,313). CD171 (also referred to as L1-CAM), was chosen as a cell surface target of interest, as there is evidence that CD171 is present in a large proportion of chemotherapy resistant cancers. CD171 traditionally is associated with both cell adhesion and motility of neural cells. Cancers expressing CD171 have been associated with poor patient survival. Expression of CD171 has been associated with a drop as high as 70% in 5-yr survival prognosis in colorectal cancer (Fang et al. 2010. Journal of Surgical Oncology 102:433-442). CD171 is not present outside of the central nervous system in adults and as such has potential applications as a targeting antibody for cancer therapeutics.

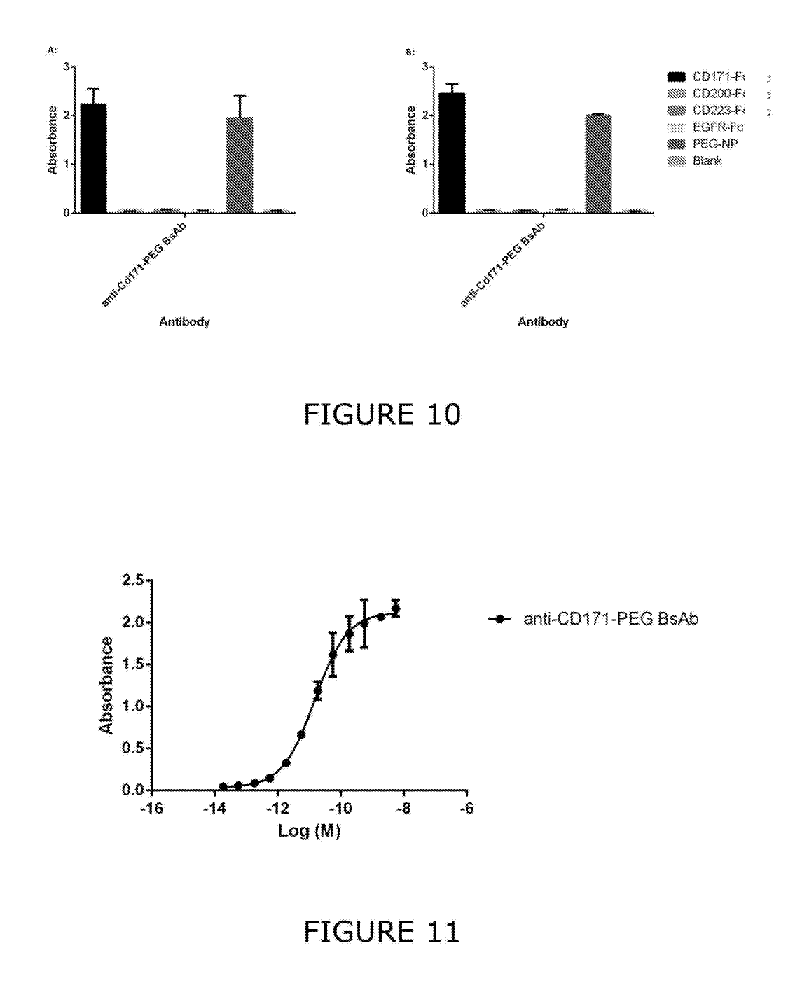

The anti-CD171-PEG BsAb in ELISA assays displays high affinity to CD171 and to the methoxyl PEG nanoparticle but not to CD200, CD227 or EGFR at a concentration of 100 μg/mL (see, FIG. 10). Binding of this BsAb to methoxyl PEG nanoparticle is not disrupted by Tween 20. This BsAb displays high affinity, estimated to be 15±4 pM, to immobilized CD171.

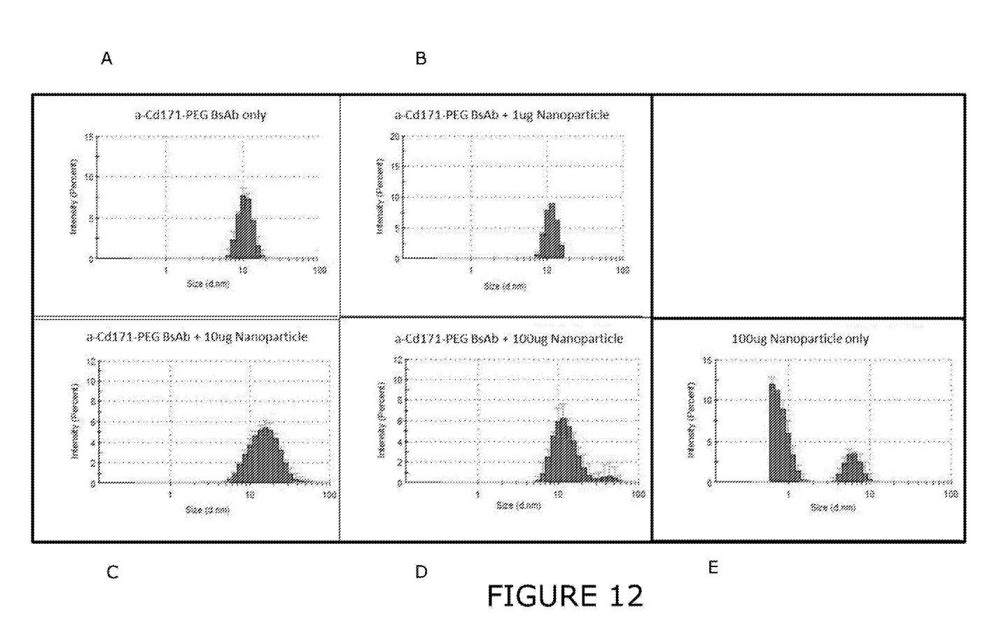

Addition of PEG-nanoparticle to anti-CD171-PEG BsAb correlates in a dose dependent manner with an increase in average particle size (FIG. 11). Anti-CD171-PEG BsAb shows and average size of 10.1 nm, anti-Cd171-PEG BsAb+1 μg nanoparticle shows and average size of 11.7 nm, anti-CD171-PEG BsAb+10 μg nanoparticle shows and average size of 15.7 nm, anti-CD171-PEG BsAb+100 μg nanoparticle shows an average size of 11.3 nm and 43.8 nm whilst 100 μg nanoparticle shows and average size of 5.9 nm (FIG. 12). This is indicative of binding of nanoparticle and anti-CD171-PEG BsAb.

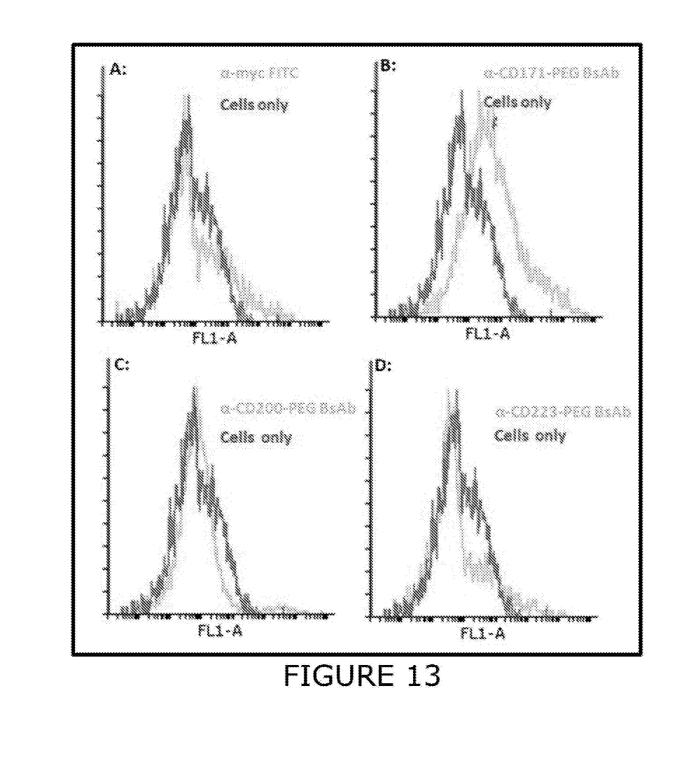

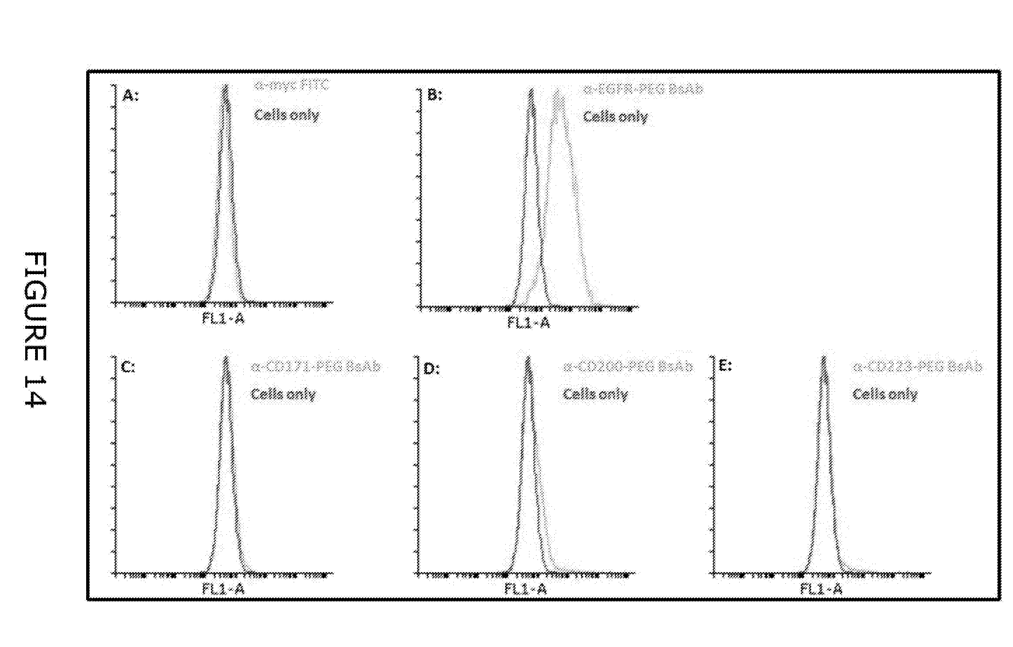

Anti-CD171-PEG BsAb bind in vitro to SKOV-3 cells that overexpress CD171 (FIG. 13) but not to MDA-MB-468 cells that overexpress EGFR but not CD171 (FIG. 14).

In this example, the term “PEG” in “anti-CD171-PEG BsAb” and “PEG-nanoparticle” refers to methoxy PEG.

### Example 5

### Anti-CD200-PEG (Samalizumab—15-2) BsAB

The anti-CD200 scFv was designed from variable regions identified from the humanized monoclonal antibody published by Bowdish et al. (2008, U.S. Pat. No. 7,408,041). CD200 (also referred to as OX-2) is associated with the stem cell-like characteristics of cells that are thought to be present in dormant cancer stem cells, which may be present at the hypoxic center of tumors. It has also been shown to be a potent tumor response suppressor. CD200 overexpression has been correlated with an increased probability of relapse following chemotherapy, and a more aggressive disease phenotype than observed in non or low expressing tumors; this furthers the hypothesis of CD200 as a cancer stem cell marker.

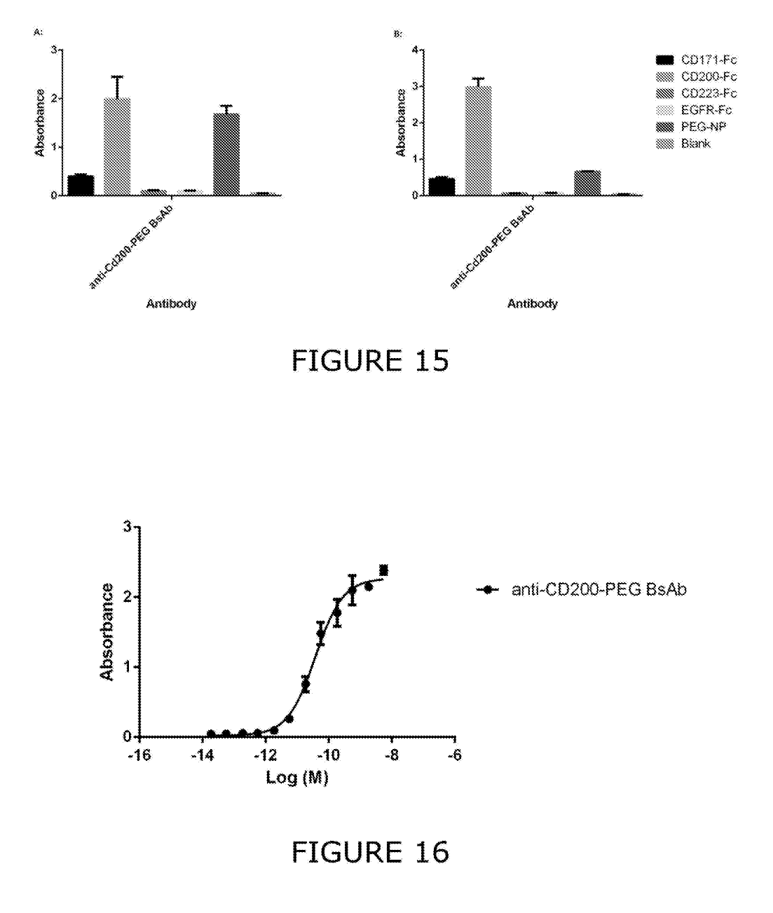

The anti-CD200-PEG BsAb in ELISA assays displays high affinity to CD200 and to the methoxyl-PEG nanoparticle but not CD171, CD227 or EGFR at a concentration of 100 μg/mL (see, FIG. 15). Binding of this BsAb to methoxyl-PEG nanoparticle is disrupted by Tween 20. The BsAb displays high affinity estimated to be 38 ±8 pM to immobilized CD200 (FIG. 16) and binds to immobilized PEG nanoparticle.

In this example, the term “PEG” in “anti-CD200-PEG BsAb” and “PEG-nanoparticle” refers to methoxy PEG.

### Example 6

### Anti-CD227-PEG (Clivatuzumab Tetraxetan—15-2) BsAb

The anti-CD227 scFv was designed from variable regions identified from the humanized monoclonal antibody with affinity to the PAM4 epitope of CD227 published by Gold et al. (2015, EP 1 521 775 B1). CD227 (also referred to as MUC-1) is thought to be one of the mucins responsible for chemotherapeutic resistance by forming a mucinous cocoon around a tumor, thus inhibiting access of immune cells and chemotherapeutics to the tumor, as well as retaining growth factors secreted by tumor associated cells. However, MUC-1 is a membrane associated protein and as such should be subject to membrane turnover allowing for internalization of mucin bound protein. Furthermore, MUC-1 has been shown to interact with both p53 and Bcl-2-Associated death promoter to inhibit apoptosis.

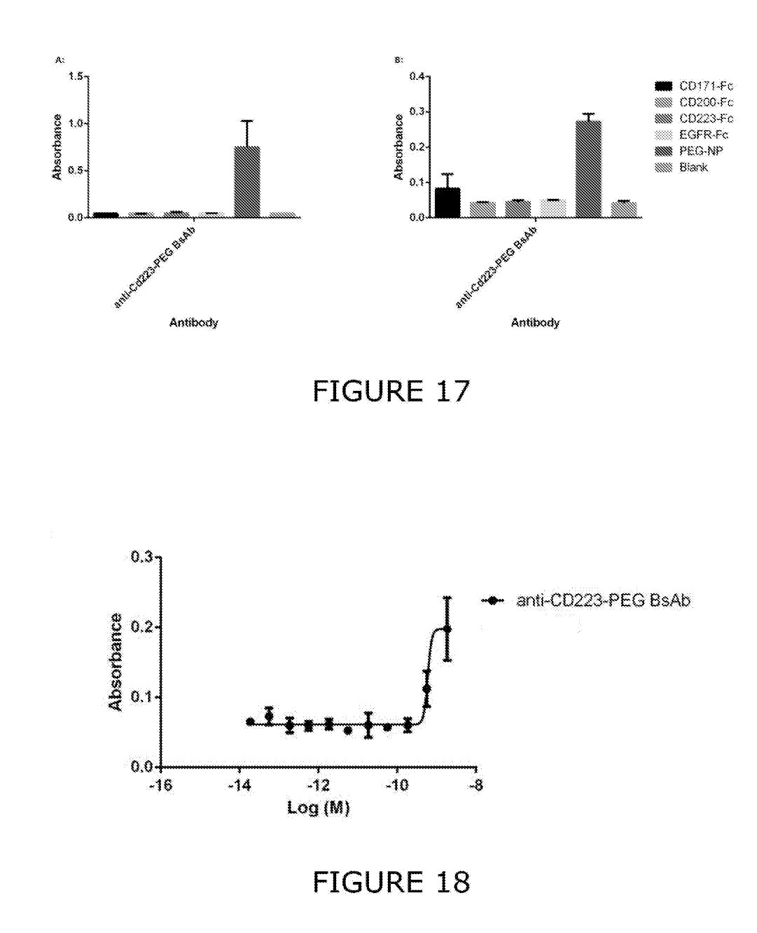

The anti-CD227-PEG BsAb in ELISA assays displays affinity to the methoxyl-PEG nanoparticle but not CD171, CD200, CD227 or EGFR at a concentration of 100 μg/mL (see, FIG. 17). Binding of this BsAb to methoxyl-PEG nanoparticle is disrupted by Tween 20. The BsAb displays some affinity estimated to be ˜60 nM to immobilized CD227 (FIG. 18).

In this example, the term “PEG” in “anti-CD227-PEG BsAb” and “PEG-nanoparticle” refers to methoxy PEG.

### Example 7

### Characterization of EGFR-mPEG BsAb-HBP Bionanomaterial

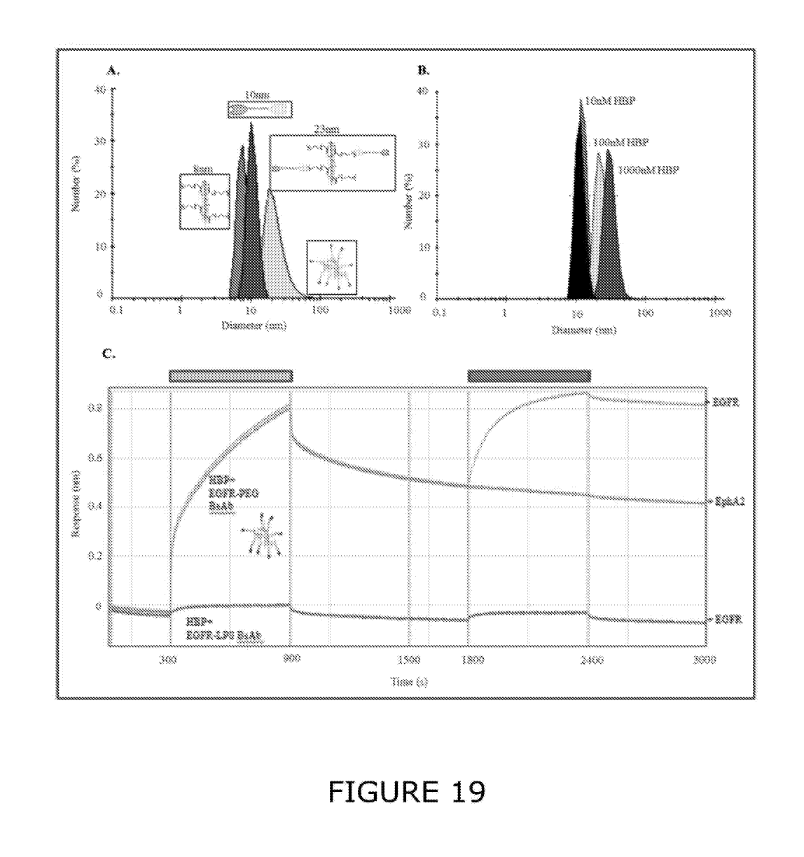

Following validation of the format and target binding of the BsAb, construction of the bio-nanomaterial was undertaken. Given the high affinity of the anti-PEG component of the BsAb, the materials were expected to self-assemble under aqueous conditions to form the hybrid bio-nanomaterials. This hybrid bio-nanomaterial formation was monitored by DLS and clearly evolved as the two components were mixed as the anti-PEG binding occurred (FIG. 19A). The HBP had a diameter of 8 nm and the BsAb had a diameter of 10 nm. Mixing of the BsAb and polymer resulted in the formation of a complex with a diameter of 23-40 nM. This implies that there is significant coverage of the HBP with BsAbs under the conditions of this experiment. To investigate the binding characteristics further, titration of different concentrations of HBP (10 nM, 100 nM and 1000 nM) with 2 pM BsAb demonstrated gradual shift towards larger particle size as multiple epitopes were bound (FIG. 19B). A bio-nanomaterial that was 28 nm in size was obtained at 1000 nM.

BLI was also utilized to confirm the binding of HBP-BsAb bio-nanomaterials to rEGFR. BsAbs were bound to HBP immobilized on biosensors (green bar; FIG. 19C), and then rEGFR was exposed to the immobilized HBP-BsAb complexes to evaluate binding capabilities of the complex (Red Bar; FIG. 19C).

rEGFR bound to the complexes whereas an alternative receptor (EphA2) (i.e., not a target of the BsAb) did not bind as evidenced by the poor response in the chromatogram (FIG. 19C). The EGFR-LPS BsAb did not bind to HBP, EGFR or EphA2 and confirms that the binding events by EGFR-mPEG are specific interactions.

### Example 8

### Bispecific Antibody Targeting of Cy5-HBP to Native EGFR on MDA-MB-468 Cells

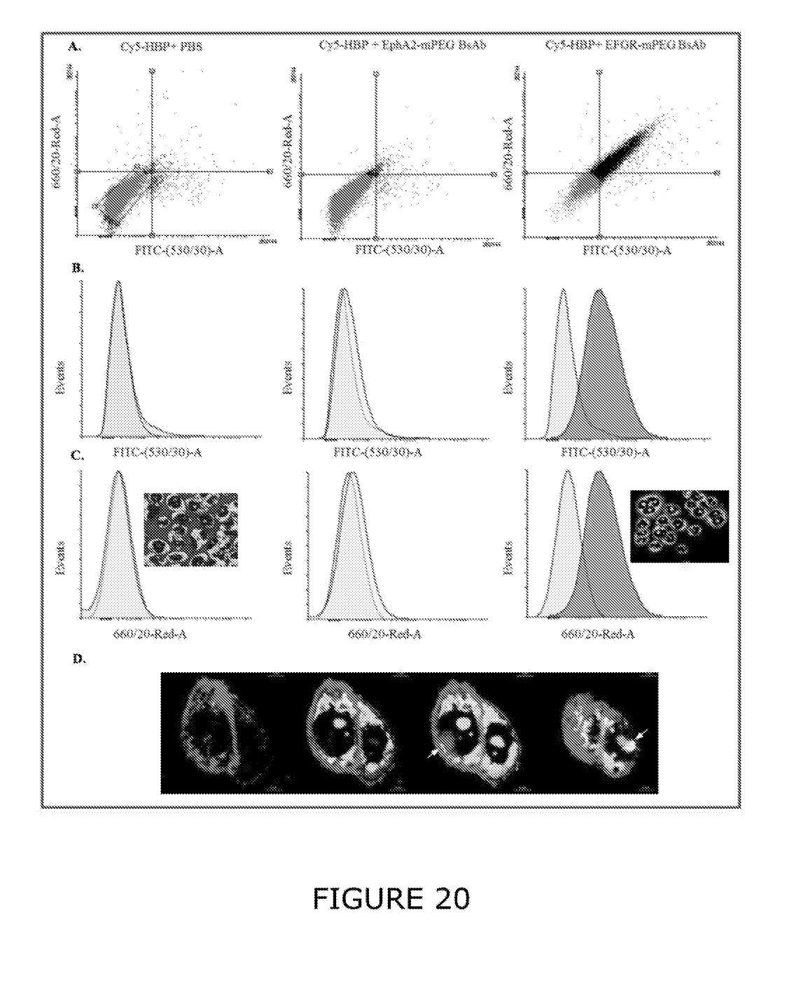

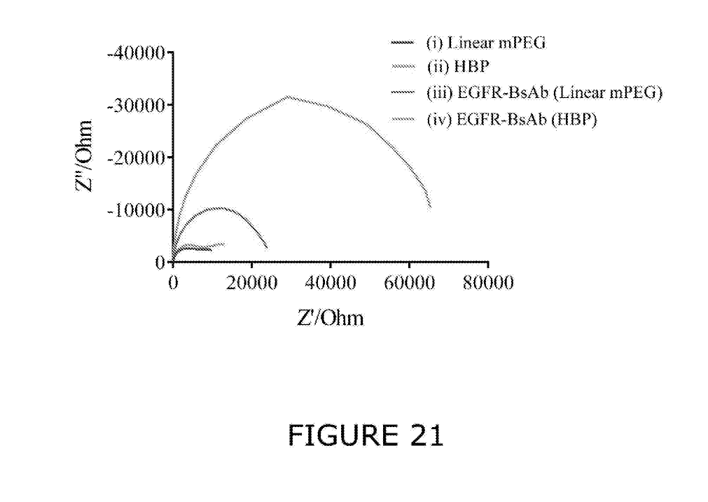

In vitro analysis of the binding efficiency of the bio-nanomaterials was monitored by both FACS and confocal microscopy. The Cy5-labeled HBP was pre-incubated with BsAbs (EGFR-mPEG, EphA2-mPEG and mesothelin-mPEG) to form a BsAb-Cy5 HBP complex. Cy5-HBP and Cy5-HBP tethered with BsAbs were incubated with different cancer cell lines and the proportion of Cy5 fluorescent cells was compared to assess the efficiency of HBP targeting when tethered with BsAbs. The EGFR-mPEG BsAb conjugated to Cy5 HBP specifically targeted native EGFR expressed on MDA-MB-468 cells (FIG. 20). Owing to the low expression levels of the particular antigens, there was no targeting of the control BsAb (EphA2-mPEG BsAb conjugated Cy5-HBP) to the MDA-MB-468 cells. To ensure that the control bio-nanomaterials were successfully formed, EphA2-mPEG BsAb conjugated Cy5-HBP was incubated with PC3 cells (which are known to overexpress EphA2) and showed high binding to these cells, whereas mesothelin-mPEG BsAb conjugated Cy5-HBP did not.

Finally, the mesothelin-mPEG BsAb effectively targeted Cy5-HBP to H226 mesothelioma cells that overexpress mesothelin. These additional experiments confirmed that the binding was specifically mediated by receptor-antigen interactions. Titration experiments using different concentrations of BsAb (200 nM, 100 nM, 50 nM, 25 nM, 12.5 nM, 6.25 nM) mixed 1:1 with different concentrations of HBP (1000 nM, 200 nM, 4 nM) indicated >90% targeting of cells using 50-200 nM BsAb.

Laser scanning confocal microscopy further demonstrated the binding of Cy5-HBP to MDA-MB-468 cells when targeted with the EGFR-mPEG BsAb (FIG. 20C). EGFR-mPEG BsAb targeted Cy5-HBP (red) accumulated at the plasma membrane, clearly staining the periphery of the cells, whereas free Cy5-HBP remained predominantly in extracellular space. Binding and subsequent internalization of the EGFR-mPEG BsAb targeted Cy5-HBP was further investigated by collecting sequential images through the z-volume of representative cells (FIG. 20D). The double stranded RNA stain Pyronin-Y (green) was used as a marker of the internal environment, thus giving an indication of the utility of BsAbs for the delivery of therapeutics to specific subcellular areas of interest. The far left image of FIG. 20D shows a region of the cell adjacent to the basal surface. Cy5-HBP fluorescence (red) appears predominantly along the cell surface although vesicular structures indicative of endocytic uptake, are present in the cytoplasm (white arrows). Progressing through the cell, more vesicular and vacuolar structures become apparent, particularly within the perinuclear space; a number of regions containing co-localized Cy5 and Pyronin-Y signals (yellow). Towards the apical surface of the observed cells, more of these structures can be seen, likely migrating from the plasma membrane further towards the cell interior. As these images were collected within a two-hr time-frame, the results suggest that the BsAb rapidly binds EGFR in vitro, being distributed across the plasma membrane and allowing for entry of the HBP into the cytoplasm through mixed mechanisms that vary in kinetics. This is demonstrated through the combination of rapid internalization events and accumulation at the cellular periphery. These observations are in agreement with the known endocytic uptake pathways for EGFR. Additionally, through comparison of Cy5-fluorescence with the Pyronin-Y channel, it is apparent that there are several pockets of the cellular milieu displaying co-localized signal and many Cy5-laden vesicles being situated adjacent to regions that show RNA metabolism. These results suggest that once the BsAb-HBP complex reaches a tumor mass in vivo, therapeutic delivery to EGFR expressing tumor cells is feasible.

### Example 9

### Attachment of BsAbs to a Polymer-Coated Transducer for Electrochemical Detection of Target Antigen

EGFR-mPEG BsAb were attached to a mPEG-coated screen-printed gold electrode (SPGE) for detection of EGFR using Faradic electrochemical impedance spectroscopy (F-EIS) as read-out. This label-free detection methodology causes an observable change in capacitance and interfacial electron transfer resistance after the layering of successive biomolecules on the electrode surface. The data are typically presented in the form of a Nyquist plot, in which Z′ and Z″ represent the real and imaginary components respectively, and the semi-circular region is proportional to the electron-transfer resistance at the electrode surface, R_{ct}. Accordingly, stepwise fabrication of an immunosensing surface and antigen capture layers, may be detected by F-EIS in the presence of a ferricyanide [Fe(CN₆)]³⁻ redox probe.

The SPGE was prepared as follows: DropSens screen-printed gold SPGE were functionalized with 1 mM mPEG (or 1 mM HBP) by incubation at 25° C. static for 1.5 hrs. 1 mM MCH was then incubated for 1 hr under the same conditions. Following monolayer formation on the sensor surface, 1 μg/mL of EGFR-mPEG BsAb was incubated on the electrode surface for 45 min. All electrochemical experiments were conducted at room temperature (25±1° C.) in a standard three-electrode electrochemical cell arrangement using an electrochemical analyzer CHI 650D (CH Instruments, Austin, Tex.), where the electrochemical cell consisted of a Au sensor as a working electrode, a Pt counter electrode, and a Ag/AgCl (3 M NaCl) reference electrode (DropSens, Spain). Electrochemical signals were measured in a 10 mM phosphate buffer solution (pH 7.4) containing 2.5 mM [Fe(CN)₆]³⁻/[Fe(CN)₆]⁴⁻ (1:1) and 0.1 M KCl. The faradic current generated by the K₃[Fe(CN)₆]/K₂[Fe(CN)₆] probe accounts on the presence of a protein.

Serum spiked with 10 ng/mL EGFR was added to the electrode and incubated for 2 hr to allow for adequate antigen diffusion to the sensor surface. A significant increase in signal was detected using mPEG or HBP functionalized SPGE coated with EGFR-mPEG BsAb, as compared to signal using mPEG or HBP functionalized SPGE controls. Of note, HBP functionalized SPGE-EGFR-mPEG BsAb bilayer produced a much stronger signal than mPEG functionalized SPGE-EGFR-mPEG BsAb bilayer, presumably due to the presence of a higher density of epitopes on HBP, relative to linear mPEG.

### Experimental

### Production of BsAb-expressing Cell Lines and Isolation of BsAbs

For the generation of stable cell lines which have the BsAb gene integrated into the chromosome of the CHO cell, 10 million CHO cells in 0.5 mL CD-CHO medium were electroporated with 10 μg plasmid DNA using a square wave protocol, 250V pulse for 30 ms in a 4 mm cuvette (BIORAD). A mock electroporation was also set up where no DNA was added to the cells. Following electroporation, the cells were transferred to 10 mL CDCHO containing 800 μg/mL of Geneticin (G418; Invitrogen) and incubated for 7 days at 37° C., 7.5% CO₂, 70% humidity. The plasmid DNA contains a neomycin gene which confers resistance of cells containing the plasmid to G418 meaning that cells without the plasmid DNA will be killed by G418. Every 5 days for three to four weeks the cells were diluted in fresh CD CHO medium containing 800 μg/mL G418. Cells were then upscaled into 125 mL suspension flasks containing CD CHO with 800 μg/mL G418 and 0.4% ACA. Stable pools were scaled up to 1L and BsAb production continued for 10-14 days.

BsAb expression and secretion from CHO cells into culture supernatant was evaluated by western blot. Briefly, supernatants from transient transfections of BsAbs were run on 4-12% Bis-Tris PAGE (Invitrogen) and the proteins transferred to PVDF membranes (BIORAD). The membranes were blocked in 2% milk-PBST (0.05% Tween-20 in 1×PBS) for 60 min, and were then probed with HRP anti-cmyc antibody (Miltenyi Biotech) diluted 1/5000 in blocking solution for 60 mins. The membranes were washed 3×5 mins in PBST and ECL substrate (Novex) was added and protein bands detected using BIORAD imaging system.

Following transfection, the cells were pelleted by centrifugation at 5250 g for 30 min and the supernatant was collected and filtered through a 0.22 μm membrane (Sartorius). The BsAbs were purified from the supernatant utilizing a 5 mL Histrap excel column (GE Healthcare), eluting the protein with 20 mM sodium phosphate, 500 mM sodium chloride and 500 mM Imidazole pH 7.4 or a 5 mL Protein L column (GE Healthcare), eluting the protein with 100 mM Glycine pH 3.0. Elution fractions were buffer exchange into phosphate buffered saline pH 7.4 using the HiPrep 26/10 column (GE Healthcare). Size exclusion chromatography using the analytical S200 column (GE Healthcare) was used to remove aggregates from BsAb preparations. The column was equilibrated in PBS+200 mM NaCl+20% ethanol, and 500 μL of sample was loaded. Ethanol was used to limit BsAb association with the column. Following fractionation of monomeric peaks, these fractions were buffer exchanged into PBS using membrane based concentrators with 10 kDa Mw cutoff (Millipore).

The final product was filtered through a 0.22 μm membrane and the concentration was determined by measuring protein absorbance at 280 nm using the Nanodrop 1000 and protein was further analyzed by SDS PAGE using 4-12% Bis-Tris gels (Invitrogen) and size exclusion HPLC using the TSK gel G3000SW column (Tosoh). HPLC was performed in the presence of 20% ethanol to prevent non-specific interactions with the column.

### Target Binding ELISA

The target binding of the BsAbs was evaluated by Indirect ELISA methods using hyperbranched mPEG (HBP), linear mPEG (Mw 2000; Polysciences) and recombinant target proteins immobilized on ELISA plates. Individual wells of a 96 well maxisorp plate (Nunc) were coated with 100 μL of 10 μg/mL of HBP or 10 μg/mL of target recombinant receptor (EphA2, VEGFR2, Mesothelin and EGFR) for 16-20 hr at 4° C. In addition, lipopolysaccharide (LPS) which is a glycan based polymer was coated at 10 μg/mL and was used as a control for testing BsAb specificity for mPEG. HBP, linear mPEG, LPS and target receptors were diluted in phosphate buffered saline (PBS pH 7.4). Following coating, the solution was decanted and 200 μL of 2% skim milk in PBST (PBS+0.05% Tween 20) was added to each well for 60 min to block non-specific binding. The blocker was decanted and 100 μL of BsAb, either in cell culture supernatant or purified protein stored in PBS, was added to each well. Each BsAb was tested in triplicate or quadruplicate wells for statistical relevance. The BsAb was incubated for 2 hr and then decanted. The wells were washed five times manually in PBST and 100 μL of HRP labeled anti-c-myc antibody diluted 1/5000 in blocker was added to each well and incubated for 30 min. The c-myc antibody was then decanted and the wells washed again five times manually with PBST. One hundred microliters of TMB was added to all wells and incubated for 15 min or until adequate color development was identified. The TMB colorimetric reaction was neutralized by adding 100 μL of 2M sulfuric acid. The colorimetric reactions in each well was analyzed at an absorbance of 450 nm using the Spectramax plate reader. Average absorbance and standard deviation was determined for each sample and the results presented as histograms using excel software.

### Competitive Binding ELISA

A competitive binding ELISA was used to determine the concentration of free HBP required to inhibit the binding of BsAb to immobilized HBP. This competitive binding assay was used to determine the interactions of HBP with BsAb in solution. Purified BsAb was diluted in PBS to 200 nM (10 μg/mL) and mixed with a 10-fold excess of HBP (2000 nM, 60 μg/mL), the same concentration (200 nM, 6 μg/mL) and 10-fold less HBP (20 nM, 0.6 μg/mL) for 60 mins. BsAb-HBP mixes were added to ELISA plates coated with 100 μL of 10 μg/mL HBP (1 μg per well) and target binding ELISA protocol was followed.

### Biolayer Interferometry (BLI)

BLI was used to determine the binding affinity constants (K_{D}) of the BsAbs for HBP and rEFGR targets. The Octet-Red (ForteBio) platform was used to evaluate binding kinetics of the BsAb for targets using 96-well black plates (Greiner BioOne). Each well was prepared with 200 μL of sample, the reactions were conducted at 30° C. and 1000 rpm agitation was used for each step. Biosensors were hydrated in 200 μL of 1×PBS (Lonza) for 10 min prior to the start of the binding assay.

Aminopropylsilane (APS) biosensors (Fortebio) which bind hydrophobic sites on various molecules were used to immobilize HBP. The assay conditions included an initial baseline step in PBS for 5 min, followed by loading 100 μg/mL HBP for 10 min, PBS baseline for 5 min, two blocking steps with 1 mg/mL BSA for 10 min each, PBS baseline for 5 min and then an association step with BsAb for 10 min followed immediately by a 10 min dissociation step in PBS.

Anti-human Fc specific biosensors (Fortebio) were immobilized with 100 μg/mL rEGFR-hFc (sinobiological) to measure BsAb binding kinetics for EGFR. Similar assay conditions were used with initial PBS baseline, EGFR-hFc immobilization, PBS baseline, BsAb association and dissociation. A global fit of a 1:1 binding model was adopted in the Octet software package to determine the binding constants (K_{D}) of BsAb for each target. BsAb was titrated at two-fold molar concentrations from 500 to 15.6 nM for HBP and 125-3 nM for EGFR. BSA at the highest concentration of BsAb used was representative of the reference sample that was subtracted from BsAb binding response to determine binding constants.

### Flow Cytometry

To determine the binding of the EGFR-mPEG BsAb to native EGFR, flow cytometry was performed using the breast cancer cell line MDA-MB-468 cells which overexpress EGFR. PC3 prostate cancer cells which overexpress the ephrin A2 (EphA2) receptor were used to test binding of the EphA2-mPEG BsAb. H226 cells which express mesothelin receptor were used to test binding of the mesothelin-mPEG BsAb. MDA-MB-468 cells (ATCC® HTB-132™), PC3 cells (PC-3 (ATCC® CRL-1435™) and H226 cells (ATCC® CRL-5826™) were cultured in 10% FCS (Hyclone) in advanced RPMI (Invitrogen) with 2 mM Glutamax (Invitrogen) at 37° C. in 5% CO₂. For flow cytometry experiments, cells were removed from tissue culture flasks using a cell scraper (Sardstedt), centrifuged at 700 rpm for 5 mins and the cell pellet was resuspended in 10% FCS-PBS to give 2×10⁶ cells/ml. 100 μL of the cell suspension was aliquoted into 1.5 mL tubes and stored on ice.

For flow experiments various concentrations of BsAb and Cy5 labeled HBP were combined to evaluate in vitro targeting. 100 μL of BsAbs (Mw 55 kDa) at concentrations of 4.0 μM, 0.8 μM, 0.4 μM, 0.2 μM, 0.1 μM, 0.05 μM and 0.025 μM in 10% FCS-PBS were mixed with 100 μL of PBS or 100 μL each of Cy5 labeled HBP (Mw 30 kDa) diluted in PBS at concentrations of 100 μM, 20 μM, 10 μM, 5 μM, 1 μM and 0.2 μM. The Cy5 HBP at these concentrations were also mixed with PBS. The reactions were incubated for 60 min at room temperature and were then added to 100 μL of cells and incubated for 1 hr at 4° C. The different concentrations of Cy5 polymer were also premixed with PBS to test polymer alone on the cells. Following incubation, the cells were centrifuged gently at 1000 rpm for 5 min, the supernatant was pipetted off and 200 μL of 10% FCS-PBS added. This wash step was repeated two more times. After the third and final wash, the supernatant was removed from the cells and the pellet was resuspended in 100 μL of FITC labeled anti-c myc antibody (Miltenyi Biotech) diluted 1/11 in 10% FCS-PBS. The antibody was incubated with the cells in the dark for 1 hr at 4 C. Following incubation, the wash steps were repeated and then the cell pellet was resuspended in 100 μL 10% FCS-PBS for analysis by flow cytometry. Cells were analyzed on the BD LSR II analyzer at QBI using FITC (530/30)-A and 660/20-Red-A optical filter settings or the Accuri C6 Flow Cytometer using FL1 (533/30 nm) and FL4 (675/25 nm) optical filter settings. Data were evaluated using flowing software or Accuri 6 based software.

### Preparation for Live Cell Imaging

Live cell imaging of the binding and internalization of the EGFR-mPEG BsAb was performed using MDA-MB-468 cells. HBPs were combined with BsAb in a 1:1 w/w ratio and incubated for 45 min prior to cell exposure, being added to 2 mL of phenol red free RPMI to give a final concentration of 6.4 μg/mL of HBP. As a control, phenol red free RPMI containing HBPs (6.4 pg/mL) was also prepared.

Experimental populations were plated in 50 mm high-resolution slide bottomed μ-dishes (Ibidi) and incubated overnight, being grown to confluence. As potential siRNA gene therapy vectors were being examined, cells were exposed to 12 μM of the double stranded ribonucleic acid stain Pyronin-Y in phenol red free RPMI (Sigma-Aldrich & Life Technologies respectively) as per the method established by Andrews and colleagues (2013, Methods and Applications in Fluorescence 1(1): 015001). The RNA stain also serving as a marker of the internal environment of cells being examined.

### Laser Scanning Confocal Microscopy

Cells were imaged using a Carl Zeiss 710 Inverted Laser Scanning Confocal Microscope housed at the Australian Nanofabrication Facility, Queensland Node. This machine being equipped with Helium-Neon and Argon lasers and a 40×1.2 NA water immersion objective. Sequential scanning was utilized to minimize bleed-through and cross-talk between fluorophores, Pyronin-Y being excited at 514 nm and Cy5-HBP at 633 nm. The collection ranges for each channel were set to 530-660 nm and 645-750 nm for Pyronin-Y and Cy5 respectively. The images were collected with a line average of four to improve image quality. Prior to imaging, the Pyronin-Y media was removed and replaced with BsAb-HBP or HBP only phenol red free RPMI. The μ-dishes were immediately transferred to the instrument and cell populations were imaged. Images were collected at 15, 30, 60 and 90 min post-exposure to either BsAb-HBP complex or HBP alone. Sequential images taken through the z-volume of representative cells were collected in order to assess internalization.

### Dynamic Light Scattering Analysis

The hydrodynamic diameter of the HBP, EGFR-mPEG and HBP+EGFR-mPEG was measured at room temperature using a dynamic light scattering device (DLS, Zetasizer Nano, Malvern, UK). For preliminary experiments 4 μM of BsAb (200 μg/mL) was mixed with 8 μM (240 μg/mL) of HBP in a 1 mL volume in PBS for 30 min at room temperature prior to analysis by DLS. HBP, BsAb and the HBP-BsAb samples were measured and the size distribution by number of particles determined. Subsequent titration experiments mixed 4 μM of BsAb with 40 nM, 400 nM and 4000 nM (4 μM) of HBP and compared particle sizes to BsAb alone.

In vivo Whole Animal Imaging

Four million MDA-MB-468 cells re-suspended in RPMI-1640 medium with 2 mM Glutamax (Invitrogen) were injected into mice. Tumors were grown in mice for 2-3 weeks. An excess of EGFR-PEG BsAb was mixed with Cy5-HBP (3:1) and incubated at room temperature for 60 min.

### Generation of HBP and Cy5-HBP

The synthesis of the HBP and Cy5-HBP was performed using the reversible addition-fragmentation chain transfer agent (4-cyano-4-phenylethylsulfanylthiocarbonyl)sulfanyl pentanoate and is described elsewhere.³⁶ In the case of the HBP, no Cy5 monomer was incorporated into the HBP.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A targeting construct represented by formula (I):
p-[α-L-λ]ₙ (I)
wherein:
p represents a polymer chain;
α-L-λ, independently for each occurrence, represents a multi-specific molecule,
wherein:
α, independently for each occurrence, represents an affinity moiety that binds with the polymer chain;
L, independently for each occurrence, is absent or represents a linker group; and
λ, independently for each occurrence, represents a targeting ligand; and
n represents an integer of at least 2,
wherein the polymer chain comprises a plurality of affinity moiety-binding partners, wherein individual affinity moiety-binding partners bind with the affinity moiety of a respective multi-specific molecule.

2. A targeting construct according to claim 1, wherein the affinity moiety-binding partners are the same.

3. A targeting construct according to claim 1, wherein the affinity moiety-binding partners are different.

4. A targeting construct according to claim 1, wherein an individual affinity moiety-binding partner comprises or is formed by one or more groups of at least one monomer residue of the polymer chain.

5. A targeting construct according to claim 1, wherein a first affinity moiety-binding partner comprises or is formed by one or more groups of at least one first monomer residue of the polymer chain and wherein a second affinity moiety-binding partner comprises or is formed by one or more groups of at least one second monomer residue of the polymer chain.

6. A targeting construct according to claim 5, wherein the first monomer residue and the second monomer residue are different.

7. A targeting construct according to claim 6, wherein the first monomer residue and the second monomer residue are the same and wherein the one or more groups of the first monomer residue are different to the one or more groups of the second monomer residue.

8. A targeting construct according to claim 5, wherein the first affinity moiety-binding partner binds with a first affinity moiety of the construct and wherein the second affinity moiety-binding partner binds with a second affinity moiety of the construct, whereby the first affinity moiety and the second affinity moiety are different.

9. A targeting construct according to claim 1, wherein an individual affinity moiety-binding partner comprises an end group of the polymer chain.

10. A targeting construct according to claim 1, wherein an individual affinity moiety-binding partner comprises a pendant group of the polymer chain.

11. A targeting construct according to claim 1, wherein the polymer chain is a homopolymer.

12. A targeting construct according to claim 1, wherein the polymer chain is a copolymer.

13. A targeting construct according to claim 12, wherein the copolymer is selected from a statistical copolymer, a random copolymer, an alternating copolymer, a periodic copolymer, a block copolymer, a radial copolymer, a graft copolymer, or combination thereof.

14. A targeting construct according to claim 1, wherein the polymer chain is linear.

15. A targeting construct according to claim 1, wherein the polymer chain is a non-linear polymer.

16. A targeting construct according to claim 15, wherein the non-linear polymer chain is selected from branched polymers, brush polymers, star polymers, comb polymers, dendrimer polymers, network polymers, cross-linked polymers, semi-cross-linked polymers, graft polymers, and combinations thereof.

17. A targeting construct according to claim 15, wherein the non-linear polymer comprises pendant cognate binding partners, individual ones of which bind with an affinity moiety of the targeting construct.

18. A targeting construct according to claim 1, wherein the polymer chain is crosslinked with another polymer chain.

19. A targeting construct according to claim 1, wherein the polymer chain comprises monomer residues derived from butadienes, styrenes, propene, acrylates, methacrylates, vinyl ketones, vinyl esters, vinyl acetates, vinyl chlorides, vinyl fluorides, vinyl ethers, vinyl pyrrolidone, acrylonitrile, methacrylnitrile, acrylamide, methacrylamide allyl acetates, fumarates, maleates, ethylenes, propylenes, tetrafluoroethylene, ethers, isobutylene, fumaronitrile, vinyl alcohols, acrylic acids, amides, carbohydrates, esters, urethanes, siloxanes, formaldehyde, phenol, urea, melamine, isoprene, isocyanates, epoxides, bisphenol A, chlorsianes, dihalides, dienes, alkyl olefins, ketones, aldehydes, vinylidene chloride, anhydrides, saccharide, acetylenes, naphthalenes, pyridines, lactams, lactones, acetals, thiiranes, episulf[iota]de, peptides, or combinations thereof.

20. A targeting construct according to claim 1, wherein the polymer chain is selected from polyamides, proteins, polyesters, polystyrene, polyethers, polyketones, polysulfones, polyurethanes, polysiloxanes, polysilanes, chitosan, cellulose, amylase, polyacetals, polyethylene, glycols, poly(acrylate)s, poly(methacrylate)s, poly(vinyl alcohol), poly(vinyl pyrrolidone), poly(vinylidene chloride), poly(vinyl acetate), poly(alkylene glycol)s such as poly(ethylene glycol) and poly(propylene glycol), polystyrene, polyisoprene, polyisobutylenes, poly(vinyl chloride), poly(propylene), poly(lactic acid), polyisocyanates, polycarbonates, alkyds, phenolics, epoxy resins, polysulf[iota]des, polyimides, liquid crystal polymers, heterocyclic polymers, polypeptides, polyacetylene, polyquinoline, polyaniline, polypyrrole, polythiophene, poly(p-phenylene), fluoropolymers, or combinations thereof.

21-73. (canceled)
